(19) United States
(12) Patent Application Publication
(10) Pub. No.: US 2004/0033608 A1
(43) Pub. Date: Feb. 19, 2004

(54) Plasmids, strains, and methods of use

(75) Inventors: Barry L. Wanner, West Lafayette, IN (US); Andreas Haldimann, Basel (CH)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN (US)

(21) Appl. No.: 10/270,176

(22) Filed: Oct. 10, 2002

Related U.S. Application Data
(60) Provisional application No. 60/375,059, filed on Apr. 24, 2002.
(60) Provisional application No. 60/328,642, filed on Oct. 10, 2001.

(51) Int. Cl. C12N 15/74

(52) U.S. Cl. 435/471

(57) ABSTRACT

The present invention provides methods for integrating a polynucleotide into genomic DNA of a microbe, plasmids that can be used to mediate the integration, and kits including such plasmids.

### CONTINUING APPLICATION DATA

This application claims the benefit of U.S. Provisional Application Serial No. 60/328,642, filed Oct. 10, 2001, and U.S. Provisional Application Serial No. 60/375,059, filed Apr. 24, 2002, which are incorporated by reference herein.

### GOVERNMENT FUNDING

The present invention was made with government support under Grant Numbers DMB9108005 and MCB-0110656, awarded by the National Science Foundation, and Grant Numbers GM57695 and GM62449, awarded by the National Institutes of Health. The United States Government has certain rights in this invention.

### BACKGROUND

Multicopy plasmids have greatly facilitated gene structure-function studies. However, the use of such plasmids can lead to high copy number artifacts especially in physiological studies. Thus, several methods have been developed for recombining genes onto bacterial chromosomes in order to study their function in single copy. Such methods are frequently used to construct novel E. coli strains that stably express foreign genes for use in both basic research and biotechnology (Boyd et al., (2000) J. Bacteriol., 182:842-847, Huang et al., (1997) J.Bacteriol., 179:6076-6083, and Martinez-Morales et al., (1999) J.Bacteriol., 181:7143-7148). However, the development of strains encoding complex metabolic or regulatory pathways poses special problems that often require manipulating many genes and expressing them individually at different levels, or under separate regulatory controls.

### SUMMARY OF THE INVENTION

The present invention represents an advance in the art of modifying microbial strains to contain one, preferably multiple, specific polynucleotides inserted into the chromosome of the microbe in single copy. Advantageously, preferred aspects of the method are easily used to insert multiple polynucleotides and the resulting strains are stable, e.g., the inserted polynucleotides do not require selection. The ability to make single copy insertions allows polynucleotides, for instance, regulatory regions and/or coding sequences, to be studied under normal physiological conditions. Moreover, the ability to produce strains containing multiple single copy insertions of different coding sequences allows the interaction of the polypeptides encoded by a coding sequence to be analyzed. Integration and retrieval occur by site-specific recombination, the original and retrieved plasmids containing the specific polynucleotides are identical. Retrieved plasmids are then propagated as plasmids for subsequent molecular analysis, or integrated directly into the chromosomes of other hosts, e.g., to test for genetic regulation, without further in vitro manipulations. In some aspects, the plasmids described herein have been useful not only for constructing specific fusions, which are integrated for studying in single-copy, but also for making fusion libraries, which are integrated en masse for screening in single-copy after which selected ones are later easily retrieved.

Accordingly, the present invention provides methods for integrating a polynucleotide into genomic DNA of a microbe. The methods includes introducing a first plasmid to a microbe. The first plasmid includes an attP site, for instance, attλ, attHK022, attP21, attP22, or attφ80. The first plasmid further includes a polynucleotide to be integrated into genomic DNA of a microbe, and the plasmid does not replicate in the microbe. The microbe includes an attB site corresponding to the attP site present on the first plasmid, and also includes a second plasmid having a coding sequence encoding an integrase that catalyzes site-specific recombination between the attP site and the attB site. The method also includes detecting the microbe that has a single copy of the first plasmid integrated into the genomic DNA of the microbe. The method may further include excising the first plasmid from the genomic DNA of the microbe.

The present invention also provides the plasmids described herein, and kits including the plasmids described herein.

### BRIEF DESCRIPTION OF THE FIGURES

FIG. 1. Structures of CRIM plasmid series. Symbols referring to genes, promoters, terminators, and attB sites are located above the lines, and restriction endonuclease sites are located below the lines. Gene symbols include: aadA (aminoglycoside adenyltransferase for spectinomycin and streptomycin resistance), bla (β-lactamase for ampicillin resistance), cat (chloramphenicol acetyl transferase), dhfr (dihydrofolate reductase for trimethoprim resistance), gen (gentamicin-3-acetyltransferase for gentamicin resistance), kan (aminoglycoside 3′-phosphotransferase for kanamycin resistance), pstS* (a mutant pstS, P₁-specific binding protein), tetA (tetracycline resistance), uidAf (the uidA2 fusion in pSK49Δ::uidA2 (Anderson, (1981) Nucleic Acids Res., 9:3015-3027)), and lacI^{q} (gene encoding lac operon repressor protein). Promoter symbols include: Ptac, Psyn1, Psyn4, ParaB, PrhaB, and PrhaS. Terminator symbols include the λ t0 and tL3 terminators, and bacterial terminator rgnB. AttB sites include: λ (attλ), HK (attHK022), P21 (attP21), φ80 (attφ80), and P22 (attP22). MCS (multiple cloning site, from pUC18 (Yanisch-Perron et al., (1985) Gene, 33:103-119)). OriR (conditional replication origin oriR_{γ}). Unique sites within the MCS of pAH68 include from left to right: SphI, PstI, SalI, XbaI, BamHI, SmaI, KpnI, SacI, and EcoRI. All sites are illustrated for the enzymes shown. Sites destroyed during construction are marked with an asterisk. Modules are flanked by SphI, EcoRI (BamHI or NdeI), NheI, NcoI, NotI, and ClaI (and/or BsaI) sites.

FIG. 2. A CRIM helper plasmid. Symbols referring to genes, promoters, terminators, and replication origin are located inside the circle, and restriction endonuclease sites are located outside the circle. bla (β-lactamase for ampicillin resistance), cI (cI857), Pr (phage λ promoter), xis (gene encoding excisionase, Xis), int (gene encoding integrase, Int), repA101 (gene encoding temperature sensitive replication protein), and oriR101 (replication origin R101). The nucleotide sequence of this plasmid is depicted at SEQ ID NO:22.

FIG. 3. Locations of chromosomal attB sites. 0, 25′, 50′, 75′ and 100′ refers to position on the chromosome in minutes.

FIG. 4. Integration, excision, and retrieval of CRIM plasmids from att_{HK022}. POP′ and BOB′ are sites for phage site-specific recombination according to the Campbell model (8, 42). P1, P2, P3, and P4 are priming sites used in PCR tests.

FIG. 5. A CRIM reporter plasmid for construction of lacZ transcriptional fusions. Primer sites routinely used to sequence inserts are indicated as up (TTGTCGGTGAACGCTCTCCT (SEQ ID NO:49), same as rgnB-f described herein) and dn (AAGTTGGGTAACGCCAGG (SEQ ID NO:50)). An x marks the attλ crossover site. The nucleotide sequence of the plasmid is depicted at SEQ ID NO:9.

FIG. 6. CRIM plasmids for construction of lacZ (pr) translational fusions (also referred to as protein fusions). Panel A shows the structure of pSK67, which is identical to pSK72 and pSK73 except for the polylinker preceding ′lacZ (pr). In-frame translational fusions can be generated by using any combination of polylinker sites. pSK72 and pSK73, but not pSK67, have also a BglII site in the polylinker. Symbols referring to genes, promoters, terminators, and replication origin are located inside the circle, and restriction endonuclease sites are located outside the circle. rgnB and tL3 (terminators); ′lacZ(pr) (gene encoding a truncated β-galactosidase); attL, attB site attλ; oriRg, (conditional replication origin oriR_{γ}); tet (tetracycline resistance). Panel B shows the sequence from the SphI site to residue thirteen (V) of normal, full-length LacZ. Lower case residues are encoded by the fusion junction. The SphI and BamHI sites are underlined. The nucleotide sequences of the plasmid pSK67 is depicted at SEQ ID NO:42.

FIG. 7. Zero-background CRIM plasmids for construction of lacZ (op) transcriptional fusions (also referred to as operon fusions). pLZ31, pLZ32, and pLZ33 are derivatives of pAH125 (FIG. 5), into which variant PshAI (XhoI), PshAI (PmlI), and PshAI (AvrII) sites were introduced. The sequences between the PstI and BamHI sites are shown. The nucleotide sequences of the plasmids are depicted at SEQ ID NO:39 (pLZ31), SEQ ID NO:40 (pLZ32), and SEQ ID NO:41 (pLZ33). Symbols referring to genes, promoters, terminators, and replication origin are located inside the circle, and restriction endonuclease sites are located outside the circle. rgnB and tL3 (terminators); ′lacZ(op) (gene encoding β-galactosidase and including operably linked ribosome binding site); attL (attB site attλ); oriRg (conditional replication origin oriR_{γ}); kan (aminoglycoside 3′-phosphotransferase for kanamycin resistance).

FIG. 8. CRIM helper plasmids. Int and Xis/Int helper plasmids for attλ are shown. The nucleotide sequences of the plasmids are depicted at SEQ ID NO:22 (pAH57), SEQ ID NO:43 (pKD15), and SEQ ID NO:44 (pKD16). cI (cI857); Pr (phage λ promoter), xis (gene encoding excisionase), int (gene encoding integrase), repA101 (gene encoding temperature sensitive replication protein),oriR101 (replication origin R101); lacIq, (gene encoding lac operon repressor protein); and araC (gene encoding L-arabinose regulator protein).

FIG. 9. Integration and retrieval of CRIM plasmids.

FIG. 10. CRIM plasmids with IPTG-inducible promoters. The nucleotide sequence of the plasmids are depicted at SEQ ID NO:32 (pLA1), SEQ ID NO:34 (pLA4), and SEQ ID NO:35 (pLA5). rgnB and tL3 (terminators); uv5i, uv5, and lacP (promoters); O2− (mutant lacO2 operator lacking repressor binding site); lacZ(op) (gene encoding β-galactosidase and including operably linked ribosome binding site); attHK (attB site attHK022); attL (attB site attλ); oriRg (conditional replication origin oriR_{γ}); cat (chloramphenicol acetyl transferase); kan (aminoglycoside 3′-phosphotransferase for kanamycin resistance).

FIG. 11. CRIM plasmids with arabinose-inducible promoters. The nucleotide sequence of the plasmids are depicted at SEQ ID NO:21 (pLA2), SEQ ID NO:36 (pLA7), SEQ ID NO:37 (pLA8), and SEQ ID NO:38 (pLA9). rgnB and tL3 (terminators); araBp7, araBp8, araBp6, and araBp3 (promoters); O2− (mutant lacO2 operator lacking repressor binding site); lacZ(op) (gene encoding β-galactosidase and including operably linked ribosome binding site); attL (attB site attλ); oriRg (conditional replication origin oriR_{γ}); kan (aminoglycoside 3′-phosphotransferase for kanamycin resistance); and orf-′araC (β-galactosidase encoding structural gene with N-terminal amino acid encoding residues and ribosome binding site).

FIG. 12. Regulation of LacI- and AraC-controlled lacZ fusions. Panel A. Δ, BW22653 (ΔlacZ); lac, BW25993 (lacI^{q} lacZ⁺); pLA1, BW26577 (UV5i-lacZ⁺); pLA4, BW26654 (lacP-lacZ⁺); and pLA5, BW26656 (uv5-lacZ⁺). BW26577, BW26652, BW26654, and BW26656 are integrants of BW22653 carrying the respective CRIM plasmid. Panel B. Δ, BW25113 (ΔlacZ); lac, BW22831 (araBp3-lacZ⁺); pLA2, BW26661 (araBp7-lacZ⁺); pLA8, BW26662 (araBp6-lacZ⁺); pLA7, BW26663 (araBp8-lacZ⁺); and pLA9, BW26664 (araBp3-lacZ⁺). BW26661 BW26662, BW26663, and BW26664 are integrants of BW25113 carrying the respective CRIM plasmid. β-gal Sp Act (nmol/min/OD), β-gal specific activity, where specific activity is equal to Activity/[2.5 (FDF)·A₄₁₀ (cells), and activity (nmoles/ml/min) is equal to 3.0 (CDF)·[A₄₁₀(tₑ)- A₄₁₀(t₀)]·8.33 (SDF)·222 (CF)/(tₑ-t₀).

FIG. 13. Structures of the ΔlacZ1229::cat and ΔlacZ1230 lacP-lacY fusions. Strains carrying these fusions were constructed as described in Table 6. The test (t1 and t2) and cat (c1 and c2) primers (t1: CGATACCGAAGACAGCTCAT (SEQ ID NO:51); t2: CAGCGGTTGGAATAATAGCG (SEQ ID NO:52); c1: TTATACGCAAGGCGACAAGG (SEQ ID NO:53); and c2: GATCTTCCGTCACAGGTAGG (SEQ ID NO:54)) were used to verify the structures by using a standard PCR strategy (Lynch and Wang, (1994) J. Mol. Biol., 236:6795-684). FRT, GAAGTTCCTATACTTTCTAGAGAATAGGAACTTCGGAATAGG (SEQ ID NO:55) in each of the fusion sequences).

FIG. 14. Nucleotide sequence of pAH55 (SEQ ID NO:1).

FIG. 15. Nucleotide sequence of pAH56 (SEQ ID NO:2).

FIG. 16. Nucleotide sequence of pAH63 (SEQ ID NO:3).

FIG. 17. Nucleotide sequence of pAH68 (SEQ ID NO:4).

FIG. 18. Nucleotide sequence of pAH70 (SEQ ID NO:5).

FIG. 19. Nucleotide sequence of pAH81 (SEQ ID NO:6).

FIG. 20. Nucleotide sequence of pAH95 (SEQ ID NO:7).

FIG. 21. Nucleotide sequence of pAH120 (SEQ ID NO:8).

FIG. 22. Nucleotide sequence of pAH125 (SEQ ID NO:9).

FIG. 23. Nucleotide sequence of pAH143 (SEQ ID NO:10).

FIG. 24. Nucleotide sequence of pAH144 (SEQ ID NO:11).

FIG. 25. Nucleotide sequence of pAH145 (SEQ ID NO:12).

FIG. 26. Nucleotide sequence of pAH150 (SEQ ID NO:13).

FIG. 27. Nucleotide sequence of pAH152 (SEQ ID NO:14).

FIG. 28. Nucleotide sequence of pAH153 (SEQ ID NO:15).

FIG. 29. Nucleotide sequence of pAH154 (SEQ ID NO:16).

FIG. 30. Nucleotide sequence of pAH156 (SEQ ID NO:17).

FIG. 31. Nucleotide sequence of pAH162 (SEQ ID NO:18).

FIG. 32. Nucleotide sequence of pCAH56 (SEQ ID NO:19).

FIG. 33. Nucleotide sequence of pCAH63 (SEQ ID NO:20).

FIG. 34. Nucleotide sequence of pLA2 (SEQ ID NO:21).

FIG. 35. Nucleotide sequence of pAH57 (SEQ ID NO:22).

FIG. 36. Nucleotide sequence of pAH69 (SEQ ID NO:23).

FIG. 37. Nucleotide sequence of pAH83 (SEQ ID NO:24).

FIG. 38. Nucleotide sequence of pAH121 (SEQ ID NO:25).

FIG. 39. Nucleotide sequence of pAH122 (SEQ ID NO:26).

FIG. 40. Nucleotide sequence of pAH123 (SEQ ID NO:27).

FIG. 41. Nucleotide sequence of pAH129 (SEQ ID NO:28).

FIG. 42. Nucleotide sequence of pAH130 (SEQ ID NO:29).

FIG. 43. Nucleotide sequence of pAH131 (SEQ ID NO:30).

FIG. 44. Nucleotide sequence of pINT-ts (SEQ ID NO:31).

FIG. 45. Nucleotide sequence of pLA1 (SEQ ID NO:32).

FIG. 46. Nucleotide sequence of pLA43 (SEQ ID NO:33).

FIG. 47. Nucleotide sequence of pLA4 (SEQ ID NO:34).

FIG. 48. Nucleotide sequence of pLA5 (SEQ ID NO:35).

FIG. 49. Nucleotide sequence of pLA7 (SEQ ID NO:36).

FIG. 50. Nucleotide sequence of pLA8 (SEQ ID NO:37).

FIG. 51. Nucleotide sequence of pLA9 (SEQ ID NO:38).

FIG. 52. Nucleotide sequence of pLZ31 (SEQ ID NO:39).

FIG. 53. Nucleotide sequence of pLZ32 (SEQ ID NO:40).

FIG. 54. Nucleotide sequence of pLZ33 (SEQ ID NO:41).

FIG. 55. Nucleotide sequence of pSK67 (SEQ ID NO:42).

FIG. 56. Nucleotide sequence of pKD16 (SEQ ID NO:43).

FIG. 57. Nucleotide sequence of pKD16 (SEQ ID NO:44).

FIG. 58. Nucleotide sequence of pSK72 (SEQ ID NO:45).

FIG. 59. Nucleotide sequence of pSK73 (SEQ ID NO:46).

FIG. 60. Nucleotide sequence of pKD137 (SEQ ID NO:47).

FIG. 61. Nucleotide sequence of pLA41 (SEQ ID NO:48).

### DETAILED DESCRIPTION OF PREFERRED EMBODIMENTS OF THE INVENTION

Plasmids

The present invention provides plasmids, referred to herein as “CRIM” plasmids, that can be used to deliver to a microbe a polynucleotide that is to be inserted into the microbe's genomic DNA. Genomic DNA includes the chromosome and non-chromosomal DNA, for instance, a plasmid (including F′ factors and R factors), present in the microbe. In some aspects, the polynucleotide may contain a coding sequence. “Polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. A “coding sequence” or “coding region” refers to a polynucleotide that encodes an mRNA and, when placed under the control of appropriate regulatory sequences, expresses the mRNA. The boundaries of a coding region are generally determined by a translation start codon at its 5′ end and a translation stop codon at its 3′ end. A “regulatory sequence” is a polynucleotide that regulates expression of a coding sequence to which it is operably linked. Nonlimiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is operably linked to a coding region when it is joined in such a way that expression of the coding region is achieved under conditions compatible with the regulatory sequence.

The CRIM plasmids may contain a conditional replication origin, preferably, a conditional replication origin that requires a transacting polypeptide for replication of the plasmid. A preferred example of a conditional replication origin is the γ origin (Kolter et al., Cell, 15, 1199-1208 (1978). The γ origin, also referred to as oriRγ, requires the transacting polypeptide, Π, for replication. Π is encoded by the pir coding sequence. Such a conditional replication origin is referred to herein as “pir-dependent.” As used herein, the term “polypeptide” refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like. An example of an oriRγ is nucleotides 56-447 of the sequence depicted at GenBank Accession Number M65025. Another preferred example of a conditional replication origin is the oriV origin, which requires the transacting polypeptide, TrfA, which is encoded by the trfA coding sequence. Such a conditional replication origin is referred to herein as “TrfA-dependent.” An example of the oriV is nucleotides 2183-2793 of SEQ ID NO:47, and 12186-12802 of the sequence depicted at GenBank Accession Number L27758, and an example of the trfA coding sequence and TrfA polypeptide are depicted at GenBank Accession Number X00713 (nucleotide 430 to 1578 encoding 43 kDa TrfA1 protein and nucleotide 721 to 1578 encoding 33 kDa TrfA2 protein (Smith and Thomas, J. Mol. Biol., 175, 251-262 (1984)). OriV can be replicated in a cell containing trfA present in single copy on the chromosome. Previously, trfA had to be supplied from the same plasmid or from a compatible plasmid (see, for instance, Haugan et al., J. Bacteriol., 174, 7026-7032 (1992), and Hasnain and Thomas, J. Gen. Microbiol., 132, 1863-1874 (1986)). Unless otherwise specified, “a,” “an,” “the,” and “at least one” are used interchangeably and mean one or more than one.

The CRIM plasmids also typically contain one phage attachment (attP) site. An attP site is a polynucleotide that provides for site-specific recombination with a corresponding bacterial attachment (attB) site. Preferably, the attP site is attλ, attHK022, attP21, attP22, or attφ80. An example of an attλ site includes nucleotides 4,543-4,938 of the plasmid depicted at SEQ ID NO:1. An example of an attHK022 site includes nucleotides 356-619 of the plasmid depicted at SEQ ID NO:4. An example of an attP21 site includes nucleotides 357-924 of the plasmid depicted at SEQ ID NO:6. An example of an attP22 site includes nucleotides 358-810 of the plasmid depicted at SEQ ID NO:16. An attP22 site is preferably greater than about 249 nucleotides in length, which is longer than the complete region of the attP22 site predicted by Leong et al. ((1985) J. Biol. Chem., 260, 4468-4477) to be sufficient for recombination. The use of the attP22 site described herein resulted in over 1,000 fold greater recombination than the attP22 site predicted by Leong et al., which had an undetectable level of recombination. An example of an attφ80 site includes nucleotides 358-837 of the plasmid depicted at SEQ ID NO:15. An attφ80 site is preferably greater than about 229 nucleotides in length, which is longer than the complete region of the attφ80 site predicted by Leong et al. ((1985) J. Biol. Chem., 260, 4468-4477) to be sufficient for recombination. The use of the attφ80 site described herein resulted in about 1,000 fold greater recombination than the attφ80 site predicted by Leong et al.

Typically, the CRIM plasmid contains a site or sites into which a polynucleotide can be inserted, typically by ligation. Preferably, a CRIM plasmid may contain a cloning region, e.g., a region permitting the easy addition of the polynucleotide that is to be integrated into the genomic DNA of a microbe. The cloning region may include several restriction endonuclease sites. Such a cloning region is often referred to in the art as a polylinker or multiple cloning region.

In some aspects of the invention, including those aspects where the polynucleotide to be integrated into the microbe's genomic DNA includes a coding region, a CRIM plasmid may include a regulatory region that will be operably linked to a coding sequence after it is added to the CRIM plasmid. An example of such a regulatory region is a promoter. Examples of promoters include constitutive promoters and regulatable promoters. Regulatable promoters may be inducible or repressible, and permit the conditional expression of an operably linked coding sequence at different levels. For instance, isopropyl β-D-galactopyranoside (IPTG) can be used to induce the expression of certain promoters. Examples of CRIM plasmids with such IPTG-inducible promoters include the uv5i promoter present at SEQ ID NO:32, the lacP promoter present at nucleotides 309-428 of the plasmid at SEQ ID NO:34, and the uv5 promoter present at nucleotides 309-428 of the plasmid at SEQ ID NO:35. L-arabinose can also be used to induce the expression of certain promoters. Examples of CRIM plasmids with such L-arabinose-inducible promoters include the araBp7 promoter present at nucleotides 289-606 of the plasmid at SEQ ID NO: 21, the araBp8 promoter present at nucleotides 283-613 of the plasmid at SEQ ID NO:36, the araBp6 promoter present at nucleotides 283-613 of the plasmid at SEQ ID NO:37, and the araBp3 promoter present at nucleotides 476-789 of the plasmid at SEQ ID NO:38. L-rhamnose can also be used to induce the expression of certain promoters. Examples of CRIM plasmids with such L-rhamnose-inducible promoters include the rhaBp3 promoter present at nucleotides 315-599 of the plasmid at SEQ IS NO:8, and the rhaBp4 promoter

| | |
|---|---|
| (GGCCTCACCTTAAATTTTTGACGCTAAAGCGC GCAAACATGG TCTTTTTTTC | (SEQ ID NO:120) |
| AAGAAAAGGC GGGAAAAAGCGGGAAATGCG GACGACATCA CACCGGCCTA | |
| TTAGTAGAAA CTGTGAACGC TATCACGTTCATCTTTGCCT TGTTGCCAGC | |
| GGCTCATTTT CCTGTCAGTA ACGAGAAGGT AGGTCTTTGA GGGCTTTTTT | |
| AGACTGTGCG CAATGACTCT AAGA). | |

In some aspects of the invention, including those aspects where the polynucleotide to be integrated into the microbe's genomic DNA includes a coding region, the cloning region may be flanked by terminators, preferably bacterial (for instance, rgnB) and/or phage (for instance, t0 and/or tL3) terminators to prevent transcriptional read-through of the coding sequence.

In some aspects of the invention, a CRIM plasmid may contain a reporter coding sequence. A reporter sequence is typically used in making a transcriptional fusion or a translational fusion between a polynucleotide of interest and the reporter coding sequence. Reporter coding sequences used to form such fusions can include the β-galactosidase (encoded by lacZ), β-glucuronidase (encoded by uidA), alkaline phosphatase (encoded by phoA) and luciferase (encoded by lux) coding sequences. Other reporter coding sequences include those encoding fluorescent polypeptides, including, for example, green fluorescent protein and red fluorescent protein.

In a transcriptional fusion, also referred to as an operon fusion, the reporter coding sequence includes a start codon at the 5′ end, and an operably linked translation initiation site providing for the translation of the mRNA encoded by the reporter coding sequence. Typically, a polynucleotide including a regulatory region, preferably a promoter, is inserted upstream of the translation initiation site such that it is operably linked to, and drives expression of, the reporter coding sequence. Examples of CRIM plasmids including a reporter coding sequence for use in making transcriptional fusions include SEQ ID NO:9, SEQ ID NO:32, SEQ ID NO:21, SEQ ID NO:34, SEQ ID NO:35, SEQ ID NO:36, SEQ ID NO:37, and SEQ ID NO:38.

In a translational fusion, also referred to as a protein fusion, the reporter coding sequence is truncated at the 5′ end, i.e., it does not include a start codon that is in-frame with the coding sequence. The nucleotides that are present do encode an active polypeptide. The truncated reporter coding sequence also does not have an operably linked translation initiation site. Typically, the polynucleotide that is inserted upstream of the reporter coding sequence includes a regulatory region, preferably a promoter, a translation initiation site, and a coding sequence truncated at the 3′ end, i.e., it does not include a stop codon that is in-frame with the coding sequence. The truncated coding sequence forms an in-frame fusion with the truncated reporter coding sequence, and the operably linked regulatory regions (e.g., promoter and translation initiation site) cause expression of the reporter coding sequence. Examples of CRIM plasmids including a reporter coding sequence for use in making translational fusions include SEQ ID NO:42, SEQ ID NO:45, and SEQ ID NO:46.

The invention includes additional CRIM plasmids for the construction of transcriptional lacZ fusions for use in the preparation of random fusion libraries. Such plasmids have zero-background cloning sites which permit during the construction of libraries the elimination of plasmids that do not contain insert DNA (see Majumder et al., Gene, 151,147-151 (1994)). Examples of such CRIM plasmids include SEQ ID NO:39, SEQ ID NO:40, SEQ ID NO:41.

Typically, a CRIM plasmid contains a coding sequence encoding a selectable marker. A “selectable marker” is a polypeptide that inhibits a compound, for instance an antibiotic, from preventing cell growth. Examples of a selectable markers include polypeptides that confer resistance to an antibiotic, including, for instance, spectinomycin, streptomycin, trimethoprim, gentamicin, kanamycin, ampicillin, chloramphenicol, and tetracycline.

Examples of CRIM plasmids include SEQ ID NO:1 or plasmids having nucleotides about 1-about 2,060 and about 4,155-about 6,664 of SEQ ID NO:1, SEQ ID NO:2 or plasmids having nucleotides about 1-about 2,060 and about 4,158-about 6,668 of SEQ ID NO:2 SEQ ID NO:3 or plasmids having nucleotides about 1-about 100 and about 1,135-about 3,663 of SEQ ID NO:3, the nucleotide sequence SEQ ID NO:4, SEQ ID NO:5 or plasmids having nucleotides about 1-about 100 and about 1,031-about 3,534 of SEQ ID NO:5, the nucleotide sequence SEQ ID NO:6, SEQ ID NO:7 or plasmids having nucleotides about 1-about 100 and about 1,135-about 3,843 of SEQ ID NO:7, the nucleotide sequence SEQ ID NO:8, SEQ ID NO:9 or plasmids having nucleotide about 1-about 148 and about 3224-about 5739 of SEQ ID NO:9, the nucleotide sequence SEQ ID NO:10, the nucleotide sequence SEQ ID NO:11, the nucleotide sequence SEQ ID NO:12, SEQ ID NO:13 or plasmids having nucleotides about 1-about 547 and about 1,238-about 3,695 of SEQ ID NO:13, SEQ ID NO:14 or plasmids having nucleotides about 1-about 255 and about 1,551-about 3,653 of SEQ ID NO:14, the nucleotide sequence SEQ ID NO:15, the nucleotide sequence SEQ ID NO:16, SEQ ID NO:17 or plasmids having nucleotides about 1-about 129 and about 1,426-about 3,527 of SEQ ID NO:17, the nucleotide sequence SEQ ID NO:18, SEQ ID NO:19 or plasmids having nucleotides about 1-about 2,060 and about 4,157-about 6,742 of SEQ ID NO:19, SEQ ID NO:20 or plasmids having nucleotides about 1-about 100 and about 2,195-about 4782 of SEQ ID NO:20, SEQ ID NO:21 or plasmids having nucleotides about 1-about 332 and about 3408-about 5948 or SEQ ID NO:21, SEQ ID NO:32 or plasmids having nucleotides about 1-about 471 and about 3547-about 5750 of SEQ ID NO:32, SEQ ID NO:33, SEQ ID NO:34 or plasmids having nucleotides about 1-about 445 and about 3521-about 5771 of SEQ ID NO:34, SEQ ID NO:35 or plasmids having nucleotides about 1-about 445 and about 3521-about 5771 of SEQ ID NO:35, SEQ ID NO:36 or plasmids having nucleotides about 1-about 615 and about 3691-about 5989 of SEQ ID NO:36, SEQ ID NO:37 or plasmids having nucleotides about 1-about 443 and about 3519-about 5814 of SEQ ID NO:37, SEQ ID NO:38 or plasmids having nucleotides about 1-about 829 and about 3905-about 6200 of SEQ ID NO:38, SEQ ID NO:39 or plasmids having nucleotides about 1-about 397 and about 3473-about 5706 of SEQ ID NO:39, SEQ ID NO:40, SEQ ID NO:41, SEQ ID NO:42 or plasmids having nucleotides about 1-about 305 and about 3356-about 5911 of SEQ ID NO:42, SEQ ID NO:45, SEQ ID NO:46, or the complements thereof.

The replication origins, reporter coding sequences, attP sites, and antibiotic resistance markers of these plasmids are flanked by restriction endonuclease sites to permit easy construction of new variants with different combinations of features. Thus, the present invention also includes combinations of the described replication origins, reporter coding sequences, attP sites, and antibiotic resistance markers.

The present invention also provides plasmids that can be used to mediate the integration of a CRIM plasmid into a chromosome, and the excision and retrieval of a CRIM plasmid from a chromosome. These plasmids are referred to herein as “CRIM helper” plasmids.

A CRIM helper plasmid typically contains a coding sequence encoding an integrase polypeptide. An example of a coding sequence encoding an integrase polypeptide that mediates the integration of a CRIM plasmid containing an attλ site is depicted at nucleotides 1,034-2,104 of SEQ ID NO:31, nucleotides 1542-2612 of SEQ ID NO:43, and nucleotides 1371-2441 of SEQ ID NO:44. An example of a coding sequence encoding an integrase polypeptide that mediates the integration of a CRIM plasmid containing an attHK022 site is depicted at nucleotides 908-1,981 of SEQ ID NO:23. An example of a coding sequence encoding an integrase polypeptide that mediates the integration of a CRIM plasmid containing an attP21 site is depicted at nucleotides 937-2,079 of SEQ ID NO:25. An example of a coding sequence encoding an integrase polypeptide that mediates the integration of a CRIM plasmid containing an attφ80 site is depicted at nucleotides 967-2,217 of SEQ ID NO:27. An example of a coding sequence encoding an integrase polypeptide that mediates the integration of a CRIM plasmid containing an attP22 site is depicted at nucleotides 974-2,137 of SEQ ID NO:29.

Also included in the present invention are CRIM helper plasmids containing a coding sequence encoding an integrase polypeptide, where the coding sequence has similarity with a coding sequence encoding an integrase polypeptide. The similarity is referred to as structural similarity and is generally determined by aligning the residues of the two polynucleotides (i.e., the nucleotide sequence of the candidate coding region and the nucleotide sequence of the coding sequence encoding an integrase polypeptide as described at SEQ ID NO:23, SEQ ID NO:25, SEQ ID NO:27, SEQ ID NO:29, SEQ ID NO:31, SEQ ID NO:43, or SEQ ID NO:44) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate coding region is the coding region being compared to the nucleotide sequence of the coding sequence encoding an integrase polypeptide (e.g., nucleotides 1,034-2,104 of SEQ ID NO:31). A candidate nucleotide sequence can be isolated from phage or a microbe, or can be produced using recombinant techniques, or chemically or enzymatically synthesized. Preferably, two nucleotide sequences are compared using the Blastn program of the BLAST 2 search algorithm, as described by Tatusova, et al. (FEMS Microbiol Lett 1999, 174:247-250), and available at http://www.ncbi.nlm.nih.gov/gorf/bl2.html. Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. Optionally, default values are used but the filter is set to off. In the comparison of two nucleotide sequences using the BLAST search algorithm, structural similarity is referred to as “identities.” Preferably, a coding sequence includes a nucleotide sequence having a structural similarity with the coding sequence encoding an integrase polypeptide as described herein of at least about 80%, more preferably at least about 90%, most preferably at least about 95% identity. Typically, a polypeptide encoded by a coding sequence having structural similarity to coding sequence encoding an integrase polypeptide has integrase activity. Whether such a polypeptide has activity can be determined by evaluating the ability of the polypeptide to mediate the integration of a plasmid containing the appropriate attP site into the correct attB site in a microbe's genomic DNA.

A CRIM helper plasmid may contain a coding sequence encoding an excisionase polypeptide. Preferably, a CRIM helper plasmid that is used for excision of an integrated CRIM plasmid contains a coding sequence encoding an excisionase polypeptide and a coding sequence encoding an integrase polypeptide. An example of a coding sequence encoding an excisionase polypeptide that mediates the excision of a CRIM plasmid integrated at an attλ site is depicted at nucleotides 910-1,128 of SEQ ID NO:22. An example of a coding sequence encoding an excisionase polypeptide that mediates the excision of a CRIM plasmid integrated at an attHK022 site is depicted at nucleotides 899-1117 of SEQ ID NO:24. An example of a coding sequence encoding an excisionase polypeptide that mediates the excision of a CRIM plasmid integrated at an attP21 site is depicted at nucleotides 903-1,139 of SEQ ID NO:26. An example of a coding sequence encoding an excisionase polypeptide that mediates the excision of a CRIM plasmid integrated at an attφ80 site is depicted at nucleotides 932-1,093 of SEQ ID NO:28. An example of a coding sequence encoding an excisionase polypeptide that mediates the excision of a CRIM plasmid integrated at an attP22 site is depicted at nucleotides 935-1,285 of SEQ ID NO:30.

Also included in the present invention are CRIM helper plasmids containing a coding sequence encoding an excisionase polypeptide, where the coding sequence has similarity with a coding sequence encoding an excisionase polypeptide. The structural similarity may be determined as described above for integrase polypeptides, however, the candidate coding region is compared to the nucleotide sequence of the coding sequence encoding an excisionase polypeptide as described at SEQ ID NO:22, SEQ ID NO:24, SEQ ID NO:26, SEQ ID NO:28, or SEQ ID NO:30. Preferably, a coding sequence includes a nucleotide sequence having a structural similarity with the coding sequence encoding an excisionase polypeptide as described herein of at least about 80%, more preferably at least about 90%, most preferably at least about 95% identity. Typically, a polypeptide encoded by a coding sequence having structural similarity to coding sequence encoding an excisionase polypeptide has excisionase activity. Whether such a polypeptide has activity can be determined by evaluating the ability of the polypeptide to mediate the excision of a plasmid that was previously integrated into a microbe's genomic DNA at the appropriate attP site.

Typically, CRIM helper plasmids contain a conditional replication origin, preferably, a temperature sensitive replication origin. Examples of temperature sensitive replication origins are known to the art. Preferred examples of temperature sensitive replication origins include low copy number mutants of pSC101 (Hahimoto and Sekiguchi, J. Bacteriol., 127, 1561-1563 (1976)).

Strains

The present invention provides microbial strains that can be used with the CRIM plasmids and CRIM helper plasmids in the methods described herein. The microbial strains include strains that can be used to permit replication of CRIM plasmids that include a pir-dependent conditional replication origin at a level of about 15 copies of the CRIM plasmid per cell. Such strains include the wild-type pir gene. Examples of this type of strain include BW23473 and BW25141. An example of the wild-type pir coding sequence is nucleotides 623-1540 of GenBank Accession Number M65025.

The microbial strains also include strains that can be used to permit replication of CRIM plasmids that include a pir-dependent conditional replication origin at a level of about 250 copies of the CRIM plasmid per cell. Such strains include the pir-116 form of the pir gene. Examples of this strain include BW25142. The pir-116 form of the pir coding sequence encodes a polypeptide that has one difference when compared to the polypeptide encoded by the wild-type pir coding sequence; the proline at amino acid 106 of the Π polypeptide is changed to leucine (Greener et al., Mol. Gen. Genet., 224, 24-32 (1990)).

Other strains that are useful in the methods described herein include strains for the retrieval of CRIM plasmids that include a pir-dependent conditional replication origin by introducing genomic DNA obtained from a microbe containing an inserted CRIM plasmid. The genomic DNA may be introduced by transformation (for instance, strain BW23828), conjugation, or transduction. In some aspects, generalized phage transduction may be used (for instance, strains BW23473 and BW25141). A strain particularly useful for the integration of a CRIM plasmid that includes an attP22 phage attachment site is BW25695.

Additional strains useful in the methods described herein and with the plasmids described herein are described in Tables 1 and 6. Other strains useful in the methods described herein and with the plasmids described herein can be constructed using methods known to the art (see, for instance, Provence and Curtiss, Gene Transfer in Gram-Negative Bacteria, In: Methods for General and Molecular Bacteriology, P. Gerhardt et al. (eds.)., American Society for Microbiology, Washington, D.C., 317-347 (1994)).

Methods of Use

The present invention provides methods of using the types of plasmids described herein. One method provides for integrating a CRIM plasmid in single copy into a chromosome. The CRIM plasmid contains a polynucleotide that has been inserting into the CRIM plasmid, typically by recombinant DNA techniques. The method includes introducing a CRIM plasmid containing the polynucleotide to a microbe. The microbe can be a gram-positive or a gram-negative. Preferably, the microbe is a member of the family Enterobacteriaceae, more preferably, E. coli, Salmonella spp., or Shigella spp. Typically, the microbe is not permissive for replication of the conditional replication origin present on the CRIM plasmid.

The microbe contains a bacterial attachment (attB) site, which may be present on the bacterial chromosome or a plasmid, for instance, an F′ factor, or an R factor. An attB site may be naturally present in the genomic DNA of a microbe, or may be inserted into the genomic DNA using routine methods known to the art. An attB site is a polynucleotide that provides for site-specific recombination with a corresponding attP site present on a CRIM plasmid. Preferably, the attB site is attλ, attHK022, attP21, attP22, or attφ80. Preferably, the microbe contains a combination of, or more preferably, each of the attB sites. The microbe contains a CRIM helper plasmid encoding an integrase that will catalyze site-specific recombination between the attB site present in the chromosome of the microbe and the corresponding attP site present on the CRIM plasmid that is introduced to the microbe. For instance, when the CRIM plasmid contains the phage attachment site attλ, the microbe contains the bacterial attachment site attλ, and the CRIM helper plasmid encodes the integrase that catalyzes the site-specific recombination between the attλ sites.

The method further includes detecting the microbe containing a single copy of the CRIM plasmid integrated into the microbe's genomic DNA. If the CRIM plasmid encodes a selectable marker, microbes may be detected by incubation of the microbe in the presence of the appropriate antibiotic. Whether the microbe contains the CRIM plasmid integrated in the chromosome in single copy can be determined by methods known in the art, including, for instance, Southern blot analysis or polymerase chain reaction (PCR). Preferably, PCR is used as described herein.

The present invention may be used to insert multiple polynucleotides into a microbe's genomic DNA. For instance, different CRIM plasmids, each containing a different polynucleotide that has been inserting into the CRIM plasmid, may be inserted at different attB sites of a microbe's chromosome. Alternatively, a library of polynucleotides can be inserted into a single attB site to result in a population of microbes containing the library of polynucleotides, where each member of the population has one polynucleotide of the library. Examples of libraries include libraries designed to identify coding sequences or to identify promoter sequences.

The polynucleotide that is inserted into a CRIM plasmid may include a coding sequence. In some aspects of the invention, the polynucleotide may include a regulatory region (for instance, a promoter, and/or a translation initiation site), and/or a coding sequence truncated at the 3′ end. The organism from which the coding sequence obtained is not intended to be limiting. For instance, the polynucleotide can be obtained from a prokaryote or a eukaryote organism, or produced using recombinant techniques, or chemically or enzymatically synthesized. Typically, when the polynucleotide includes a coding sequence and is obtained from a eukaryotic organism, the coding sequence does not contain introns. For instance, a coding sequence from a eukaryotic organism can be obtained by reverse transcription of an mRNA obtained from a eukaryotic organism.

The present invention also provides other methods including excising a CRIM plasmid that has been inserted into a microbe's chromosome. This excision can be done, for instance, to verify that a phenotype is the result of a coding sequence present on the integrated CRIM plasmid, or retrieve a single integrated CRIM plasmid or a population of integrated CRIM plasmids for further analysis. The retrieval of an integrated CRIM plasmid can be accomplished by obtaining genomic DNA from a microbe containing an integrated CRIM plasmid, and introducing the DNA to a microbe that permits replication of the CRIM plasmid and contains a coding sequence encoding the appropriate integrase and the appropriate excisionase. For example, the genomic DNA can be obtained by using a generalized transducing phage to prepare a lysate. Preferably, the generalized transducing phage is P1, more preferably, P1kc. Alternatively, the genomic DNA can be obtained by isolating genomic DNA, manipulating it to produce fragments of from about 10 kilobases to about 90 kilobases in length, and introducing the fragments to a microbe that permits replication of the CRIM plasmid and contains a coding sequence encoding the appropriate integrase and the appropriate excisionase.

Kits

The present invention also provides kits for integrating a polynucleotide into a microbe's genomic DNA and, optionally, for excising such a polynucleotide from a microbe's genomic DNA. The kit includes one or more of the CRIM plasmids described herein, and one or more of the CRIM helper plasmids described herein in a suitable packaging material. Optionally, the kit includes microbial strains useful in practicing the methods described herein. Instructions for use of the packaged plasmids are also typically included. The present invention also includes kits for the production of libraries, including, for instance, lacZ fusion libraries.

As used herein, the phrase “packaging material” refers to one or more physical structures used to house the contents of the kit. The packaging material is constructed by well known methods, preferably to provide a sterile, contaminant-free environment. The packaging material may include a label which indicates that the plasmids can be used for integrating a polynucleotide into a microbial chromosome. In addition, the packaging material may contain instructions indicating how the materials within the kit are employed to integrate a polynucleotide into a microbial chromosome. As used herein, the term “package” refers to a solid matrix or material such as glass, plastic, paper, foil, and the like, capable of holding within fixed limits a polypeptide or a microbe. Thus, for example, a package can be a plastic vial used to contain a plasmid or a microbe.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Construction and Evaluation of CRIM Plasmids and Strains

Materials and Methods

Media and culture conditions. Luria-Bertani (LB) broth (without glucose), tryptone-yeast extract (TYE) agar (pH 7.0) and glucose M63 agar were used as complex and minimal media (Wanner (1994) In K. W. Adolph (ed.), Methods in Molecular Genetics 3:291-310). SOB and SOC were prepared as described elsewhere (Miller (1992) A short course in bacterial genetics: a laboratory manual and handbook for Escherichia coli and related bacteria, Cold Spring Harbor Laboratory Press, Plainview). To maintain plasmids, antibiotics (from Sigma, St. Louis, Mo.) were added as follows: ampicillin at 100 μg/ml, chloramphenicol at 25 μg/ml, gentamicin at 15 μg/ml, kanamycin at 50 μg/ml, spectinomycin and streptomycin (together) at 100 μg/ml, trimethoprim at 300 μg/ml, or tetracycline at 12.5 μg/ml. Single copy integrants were selected using chloramphenicol at 6 μg/ml, gentamicin at 5 μg/ml, kanamycin at 10 μg/ml, spectinomycin and streptomycin (together) at 35 μg/ml, trimethoprim at 25 μg/ml, or tetracycline at 8 μg/ml. Proper medium pH was typically important for selection of gentamicin-resistant (Gm^{R}) integrants. Complex media (TYE) were used for selection of all resistances except trimethoprim, for which minimal media were used. Following primary selection, integrants were routinely maintained in the absence of antibiotics.

Bacteria. All strains are derivatives of E. coli K-12. Normal (self-replicating) plasmids were propagated in DH5α (Fisher et al. (1995) J.Biol.Chem. 270:23143-23149), BW5328, or BW25141. Strains from this laboratory are described Table 1. The DE(araBAD)567 and DE(rhaBAD)568 mutations correspond to the ΔaraBAD_{AH33} and ΔrhaBAD_{LD78} alleles (Haldimann et al. (1998) J.Bacteriol. 180:1277-1286), respectively. The adjacent rrnB3 ΔlacZ4787 mutations were previously called rrnB_{T14} ΔlacZ_{WJ16} (Haldimann et al. (1998) J.Bacteriol. 180:1277-1286). The ΔendA9 allele corresponds to the ΔendA8::tetAR mutation (from BT333; (Cherepanov et al. (1995) Gene 158:9-14)) after Flp-mediated excision of tetAR with pCP2010 (Cherepanov et al. (1995) Gene 158:9-14). The recD1014 mutation originated from V355 (obtained from G. C. Walker, Department of Biology, MIT, Cambridge, Mass.); (Shevell et al. (1988) J.Bacteriol. 170:3294-3296). The attP22(EcoB) allele refers to the attP22 site of E. coli B, which had been introduced into BW25368 (proA::Tn10) by using P1kc grown on NC3 (E. coli B/r hsdR; also called BW9688 (Wanner et al. (1990) J.Bacteriol.172:1186-1196) by selecting proline-independent transductants to make BW25676. Our standard “wild-type” E. coli K-12 strain is BD792 (Wanner, B. L. (1983) J.Mol.Biol.166:283-308) which is a direct F⁻ descendant of W1485 (Bachmann, B. J. (1996) In F. C. Neidhardt et al. (eds.), Escherichia coli and Salmonella typhimurium cellular and molecular biology. 2460-2488). The rph-1 allele refers to the rph frameshift mutation (Jensen, K. F. (1993) J.Bacteriol. 175:3401-3407), which is also present in E. coli BD792. Strain BD792, like both its parent W1485 and the “wild-type” E. coli K-12 EMG2 (Kaasen et al. (1992) J.Bacteriol. 174:889-898), carries the rpoS396(Am) allele. Several strains were therefore made rpoS⁺. This was done in two steps. A strain was first made tetracycline- (Tc^{R}) and kanamycin-resistant (Km^{R}) by using P1kc grown on ZK1001 (cysC95::Tn10 rpoS::kan; obtained from R. Kolter, Department of Microbiology and Molecular Genetics, Harvard Medical School, Boston, Mass.)). A resultant Cys⁻ transductant was then made cysteine-independent and kanamycin-sensitive by using P1kc grown on MG1655 (obtained from the Coli Genetics Stock Center, Yale University, New Haven, Conn.). E. coli K-12 strains BW25113, BW25141, BW25142, and BW25695 are descendants of BD792 derivatives that were made rpoS⁺.

TABLE 1
Bacterial strains

| Strain^{a} | Genotype^{b} | Pedigree^{c} | Reference or derivation^{d} |
|---|---|---|---|
| BW37 | IN(rrnD-rrnE)1 tna bglR::IS trpR ilv | W3110 | (Wanner (1994) In K. W. |
| | rpsL rph-1 | via BW33 | Adolph (ed.), Methods in |
| | | | Molecular Genetics, 3: 291-310); |
| | | | (Wanner et al. (1982) |
| | | | J. Mol. Biol. 158: 347-363) |
| BW5328 | Δ(lacIZYA argF)_{U169} rph-1 | BD792 | (Wanner (1987) J. Bacteriol. |
| | rpoS396(Am) recA1 robA1 creC510 | via | 169: 2026-2030.8) |
| | hsdR514 | BW5206 | |
| BW23473 | Δ(lacIZYA argF)_{U169} rph-1 | BD792 | (Haldimann et al. (1997) |
| | rpoS396(Am) robA1 creC510 hsdR514 | via | J. Bacteriol. 179: 5903-5913); |
| | ΔendA9 uidA(ΔMluI)::pir(wt) recA1 | BW23438 | (Haldimann et al. (1996) |
| | | | Proc. Natl. Acad. Sci. U.S.A. |
| | | | 93: 14361-14366) |
| BW23838 | lacI^{q} rrnB3 ΔlacZ4787 ΔphoBR580 | BD792 | Srl⁺ with P1kc on BW8078 |
| | ΔcreABCD154 hsdR514 Δ(pta ackA | via | (Haldimann et al. (1998) |
| | hisQ hisP)_{TA3516} phn(EcoB) | BW23832 | J. Bacteriol. 180: 1277-1286.5) |
| | DE(araBAD)567 DE(rhaBAD)568 rph- | | |
| | 1 rpoS396(Am) uidA(ΔMluI)::pir(wt) | | |
| | ΔendA9 recD1014 recA1) | | |
| BW24249 | lacI^{q} rrnB3 ΔlacZ4787 ΔphoBR580 | BD792 | Srl⁺ with P1kc on BW8078 |
| | ΔcreABCD154 hsdR514 Δ(pta ackA | via | |
| | hisQ hisP)_{TA3516} phn(EcoB) | BW24217 | |
| | DE(araBAD)567 DE(rhaBAD)568 | | |
| | rpoS396(Am) rph-1 ΔendA9 galU95 | | |
| | uidA(ΔMluI)::pir(wt) recA1 | | |
| BW24304 | lacI^{q} rrnB3 ΔlacZ4787 ΔphoBR580 | BD792 | Srl⁺ with P1kc on BW8078 |
| | ΔcreABCD154 hsdR514 Δ(pta ackA | via | |
| | hisQ hisP)_{TA3516} phn(EcoB) | BW24296 | |
| | DE(araBAD)567 DE(rhaBAD)568 rph- | | |
| | 1 rpoS396(Am) ΔendA9 galU95 | | |
| | uidA(ΔMluI)::pir-116 recA1 | | |
| BW24320 | lacI^{q} rrnB3 ΔlacZ4787 ΔphoBR580 | BD792 | Cys⁺ with P1kc on MG1655; |
| | ΔcreABCD154 DE(araBAD)567 | via | (Lessard et al. (1998) |
| | DE(rhaBAD)568 rph-1 | BW24310 | Chemistry & Biology 5: 489-504.5) |
| BW25113 | lacI^{q} rrnB3 ΔlacZ4787 hsdR514 | BD792 | Pro⁺ with P1kc on |
| | DE(araBAD)567 DE(rhaBAD)568 rph-1 | via | BW24321; (Datsenko et al. |
| | | BW25083 | (2000) |
| | | | Proc. Natl. Acad. Sci. U.S.A. |
| | | | 97: 6640-6645); (Lessard et |
| | | | al. (1998) Chemistry & |
| | | | Biology 5: 489-504) |
| BW25141 | lacI^{q} rrnB3 ΔlacZ4787 hsdR514 | BD792 | Srl⁺ with P1kc on BW8078; |
| | DE(araBAD)567 DE(rhaBAD)568 | via | (Datsenko et al. (2000) |
| | ΔphoBR580 rph-1 galU95 ΔendA9 | BW25140 | Proc. Natl. Acad. Sci. U.S.A. |
| | uidA(ΔMluI)::pir(wt) recA1 | | 97: 6640-6645); (Lessard et |
| | | | al. (1998) Chemistry & |
| | | | Biology 5: 489-504.5) |
| BW25142 | lacI^{q} rrnB3 ΔlacZ4787 hsdR514 | BD792 | Srl⁺ with P1kc on BW8078 |
| | DE(araBAD)567 DE(rhaBAD)568 | via | |
| | ΔphoBR580 rph-1 galU95 ΔendA9 | BW25137 | |
| | uidA(ΔMluI)::pir-116 recA1 | | |
| BW25695 | lacI^{q} rrnB3 ΔlacZ4787 hsdR514 | BD792 | Pro⁺ with P1kc on BW25676 |
| | attP22(EcoB) DE(araBAD)567 | via | |
| | DE(rhaBAD)568 rph-1 | BW25367 | |

^{a}All strains are λ⁻ and F⁻.
^{b}All known mutations are indicated.
^{c}The ancestral strain from another lab and its immediate ancestor in this lab are given.
^{d}Additional information is given in Materials and Methods.

CRIM (oriR_{γ}) plasmids were propagated at medium copy number (about 15 per cell) in BW23473, BW24249 or BW25141, or at high copy number (about 250 per cell) in BW23474, BW24304, or BW25142. As standard “wild-type” hosts, BW25113 and BW25695 (like BW25113, except attP22(EcoB)) were used. CRIM plasmids were retrieved from integrants of BW24320 by using helper plasmid transformants of BW23473 (pir⁺ recA) or BW25141 (pir⁺ recA) when using P1kc transduction (P1-Int-Xis (PIX) cloning (Haldimann et al. (1996) Proc.Natl.Acad.Sci.USA 93:14361-14366), or helper plasmid transformants of BW23838 (pir⁺ recD) when using transformation (see below). Strain BW37 was used as recipient for selection of Ilv⁺ transductants when determining the Ilv⁺ transducing titer of P1kc lysates.

Molecular biology methods. PCR fragments for cloning were generated by using VENT (New England Biolabs, Beverly, Mass.) or Pfu DNA polymerase (Stratagene, La Jolla, Calif.) and oligonucleotide primers (from IDT Inc., Coralville, Iowa). Other enzymes were from New England Biolabs or Promega (Madison, Wis.). QIAGEN (Hilden, Germany) products were used for isolation of plasmid DNA, extraction of DNA fragments from agarose gels, and purification of PCR fragments.

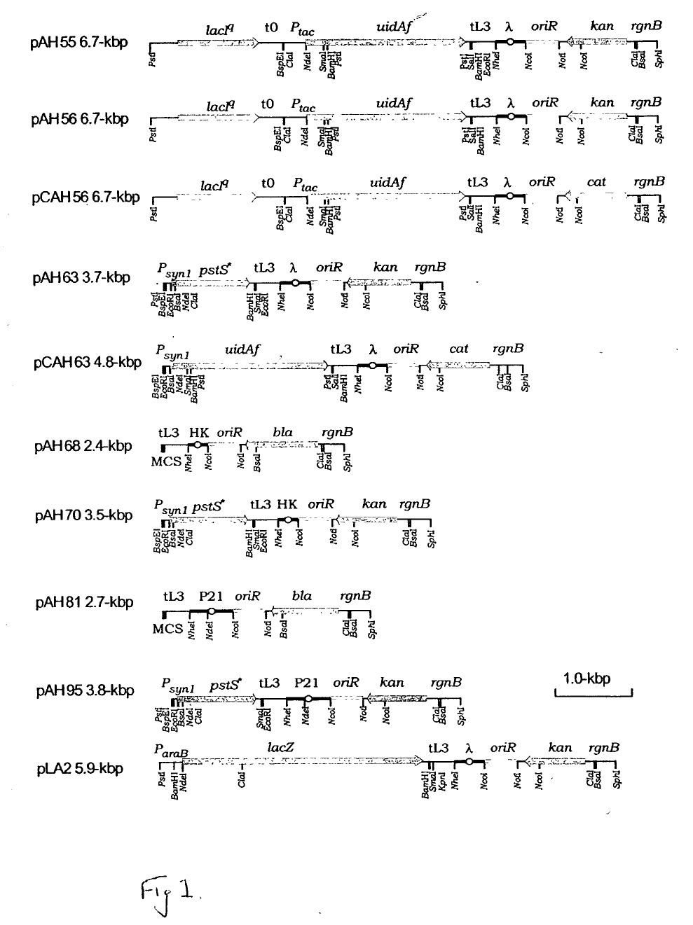

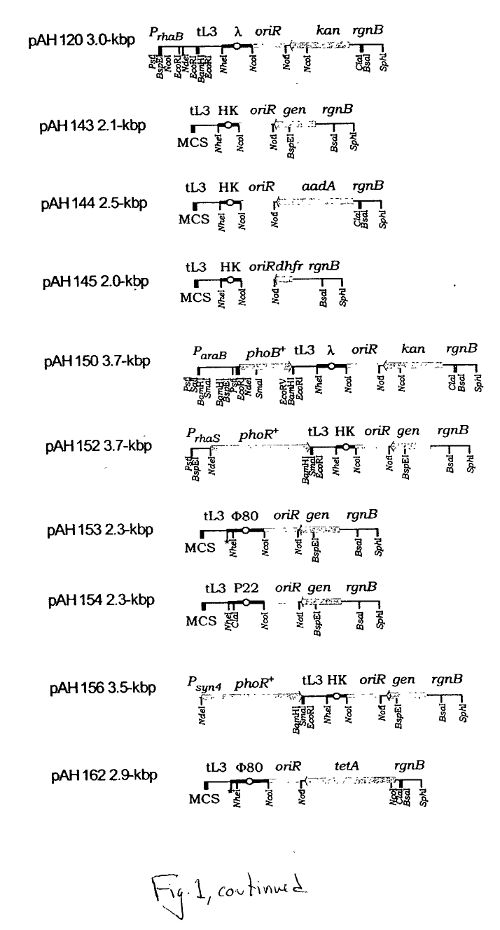

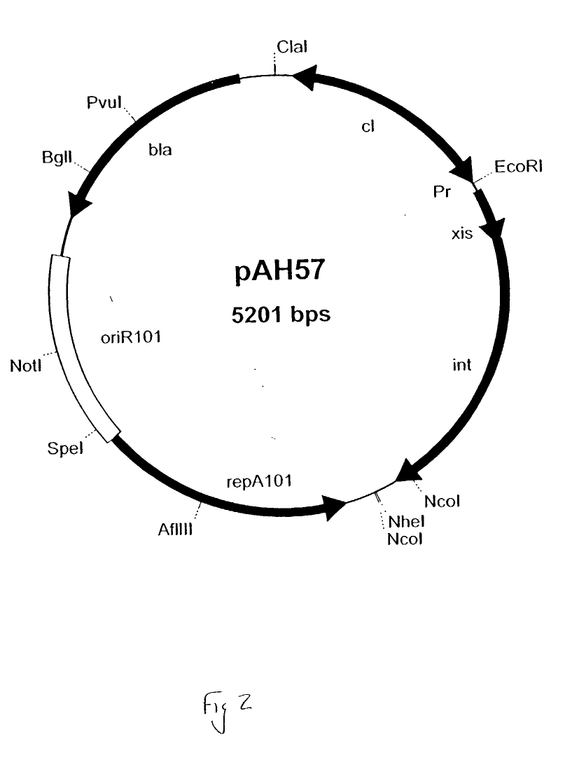

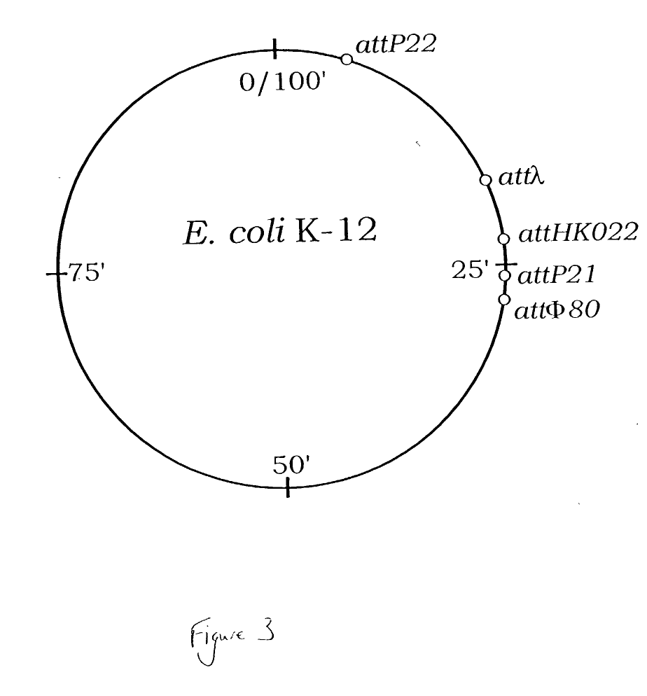

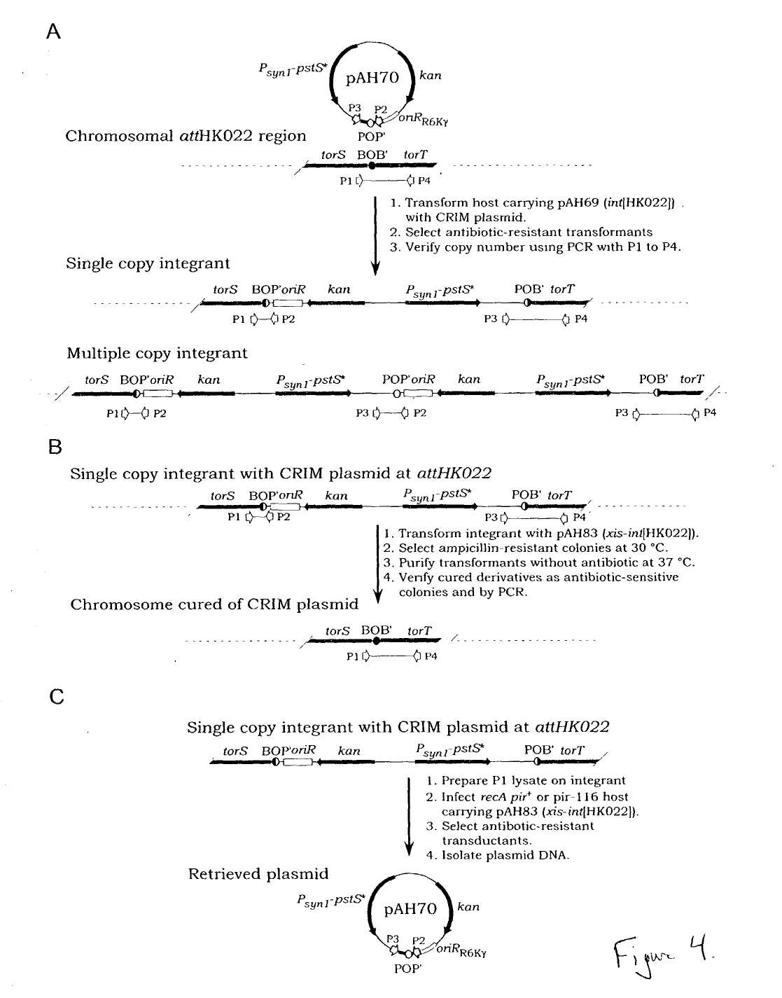

Plasmids. CRIM (FIG. 1) and CRIM helper plasmids (FIG. 2, Table 2) were assembled by standard techniques. Various fragments were subcloned directly or cloned as PCR-generated fragments containing restriction site extensions (Table 3). The bla, cat, kan, tet, and oriR_{γ} segments are from pANTSγ, pCANTSγ, pKANTSγ, and pTANTSγ (Pósfai et al. (1994) Nucleic Acids Res. 22:2392-2398); obtained from M. Koob (University of Minnesota, Minneapolis, Minn.); the attλ (with a destroyed NdeI site) and lacI^{q}-P_{tac} segments are from pCANTSγΔNdeI and pCANTSγlacI^{q}SLP (Lynch et al. (1994) J.Mol.Biol. 236:6795-684); both obtained from A. S. Lynch (Department of Microbiology and Molecular Genetics, Harvard Medical School, Boston, Mass.); the lacZ cassette in pAH125 is from pCS3 (Metcalf et al. (1993) Gene 129:17-25); the promoterless uidAf cassette is from pWM3 (Metcalf et al. (1993) Gene 129:17-25); the P_{araB} fragment in pAH150 is from pAH31 (Haldimann et al. (1998) J.Bacteriol. 180:1277-1286), P_{rhaB} in pAH120 is from pLD78 (Haldimann et al. (1998) J.Bacteriol. 180:1277-1286). The lacZ gene in pLA2 was constructed in a series of steps that involved introducing an NdeI site overlapping its Met start codon and eliminating a native NdeI site; the lacZ gene was generated using pOD (Müller et al. (1996) J.Mol.Biol. 257:21-29) as template, so it has a mutated lacO2 region; the P_{araB} fragment in pLA2 was generated as a PCR fragment (Table 3). Our initial CRIM plasmid pAH55 is a derivative of pKANTSγ in which we introduced a mutated attλ segment (lacking its native NdeI site), lacI^{q}, and uidAf segments in sequential steps. The CRIM helper plasmids were assembled using pINT-ts (Hasan et al. (1994) Gene 150:51-56) as backbone.

TABLE 2
CRIM helper plasmids

| Plasmid^{a} | Function(s) |
|---|---|
| pINT-ts | Int_{λ} |
| pAH57 | Xis & Int_{λ} |
| pAH69 | Int_{HK022} |
| pAH83 | Xis & Int_{HK022} |
| pAH121 | Int_{P21} |
| pAH122 | Xis & Int_{P21} |
| pAH123 | Int_{Φ80} |
| pAH129 | Xis & Int_{Φ80} |
| pAH130 | Int_{P22} |
| pAH131 | Xis & Int_{P22} |

^{a}The structure of an example is shown in FIG. 2.

TABLE 3

Oligonucleotide primers used for plasmid constructions

| | | |
|---|---|---|
| Region | Template | Sequence^{a} |

| | | | |
|---|---|---|---|
| aadA | pWM5 | aadA-5′:GCAATCGATACGGATGAAGGCACGAACC; | (SEQ ID NO:56) |
| | | aadA-3′:GCAGCGGCCGCTCGGCTTGAACGAATTGTTAG | (SEQ ID NO:57) |
| dhfrIIb | p34E-Tp | dhfrIIb-5′:GCAGGCGCCACGAACCCAGTTGACATAAGC; | (SEQ ID NO:58) |
| | | dhfrIIb-3′:GCAGCGGCCGCTTAGGCCACACGTTCAAGT | (SEQ ID NO:59) |
| gen | pBBR1MCS-5 | gen-5′:GCAGGCGCCCACGAACCCAGTTGACATA; | (SEQ ID NO:60) |
| | | gen-3′:GCAGCGGCCGCCTTGAACGAATTGTTAGG | (SEQ ID NO:61) |
| P_{araB} | pBAD33 | P_{araB-}5′:AACTGCAGCGCCATTCAGAGAAGAAACCAA; | (SEQ ID NO:97) |
| | | P_{araB}-3′:GCAGTCGACCATATGAATTCCTCCATCCAAAAAAACGGGTATGGAGA | (SEQ ID NO:62) |
| P_{rhaS} | pAH151 | P_{rhaS}:5′:GCATCCGGAAATTCGCGACCTTCTCG; | (SEQ ID NO:63) |
| | | P_{rhaS}:3′:GCATCTAGAGCATATGGGCCTCCTGATGTCGTC | (SEQ ID NO:64) |
| P_{syn1}*pstS* | pLD81 | P_{synl-}5′:GCATCCGGAACTAGTGTCTTCAAGAATTCTAGG; | (SEQ ID NO:65) |
| | | pstS-3′: CGGGATCCACGCGTTTACAAAGTC | (SEQ ID NO:66) |
| xis_{λ} | λ | xis₈₀ -5′:CCGGAATTCTTGCGTGTAATTGCGGAGAC; | (SEQ ID NO:67) |
| | | xis_{λ}-3′:GGAAGATCTCCTTCGAAGGAAAGACCTGATGC | (SEQ ID NO:68) |
| attP_{HK022} | pEY109 | attP_{HK022}#1:GCAGCTAGCTAATGCTCTGTCTCAGGTC; | (SEQ ID NO:69) |
| | | attP_{HK022}#2:GCACCATGGGACAAAATTGAAATCG | (SEQ ID NO:70) |
| int_{HK022} | pEY109 | int_{HK022}-5′:GCACCATGGTAAGTAGGTCATTATTAGTC; | (SEQ ID NO:71) |
| | | int_{HK022}-3′:GCAGAATTCGTAGCCTTTTGAAGAGG | (SEQ ID NO:72) |
| xis-int_{HK022} | pEY1O9 | xis_{HK022}-5′:GCAGAATTCTGCGGAGACTTTGC; | (SEQ ID NO:73) |
| | | int_{HK022}-3′:see above | |
| attP_{P21} | pBS1attP6-1 | attP_{P21}#1:GCACCATGGAATGACCGACCGATA; | (SEQ ID NO:74) |
| | | attP_{P21}#2:GCAGCTAGCATAAGGCCTCGCAA | (SEQ ID NO:75) |
| int_{P21} | pBS1attP6-1 | int_{P21}-5′:GCAGAATTCAGAACCGCAACTCCCAA; | (SEQ ID NO:76) |
| | | int_{P21}-3′:GCACCATGGATAACGGGCGTATAACA | (SEQ ID NO:77) |
| xis-int_{P21} | pBS1attP6-1 | xis_{P21}-5′:GCAGAATTCGCAGCTAAGAGGAGGAC; | (SEQ ID NO:78) |
| | | int_{P21}-3′:see above | |
| attPP22s | pJL110 | attP_{P22}#1:GCAGCTAGCCGTTGTTACCGATCAAT; | (SEQ ID NO:79) |
| | | attP_{P22}#2:GCACCATGGAAGGCACGAATAATACG | (SEQ ID NO:80) |
| attPP22 | P22 | attP_{P22}#3:GCAGCTAGCCATTATGAAAATCAGCGGATTCGGA; | (SEQ ID NO:81) |
| | | attP_{P22}#4:CATGCCATGGAATCACCTGACTGAACATGCTCGAC | (SEQ ID NO:82) |
| int_{P22} | P22 | int_{P22}-5′:GCAGAATTCCACCACACGACAAGCCT; | (SEQ ID NO:83) |
| | | int_{P22}-3′:GCACCATGGACTCCTATTATCGGCAC | (SEQ ID NO:84) |
| xis-int_{P22} | P22 | xis_{P22}-5′:GCAGAATTCCTACGACATGCCTAACG; | (SEQ ID NO:85) |
| | | int_{P22}-3′:see above | |
| attP_{χ80s} | pJL10 | attP_{χ80}#1:AACCGGTGAATCACGAC; | (SEQ ID NO:86) |
| | | attP_{χ80}#2:GCACCATGGACATCCTTGAAAGCCTG | (SEQ ID NO:87) |
| attP_{χ80} | pJL10 | attP_{χ80}#3:GCATCTAGAGGTGAATCACGACAAAGCGTATC; | (SEQ ID NO:88) |
| | | attP_{χ80}#4:CATGCCATGGGCCCGTGCGAATCAGAAATAAT | (SEQ ID NO:89) |
| int_{χ80} | pJL10 | int_{χ80}-5′:GCAGAATTCGCTAGCAACCCTTATCCAGCA; | (SEQ ID NO:90) |
| | | int_{χ80}-3′:GCACCATGGCAATCAAAGAGTGGGAG | (SEQ ID NO:91) |
| xis_{χ80} | pJL10 | xis_{χ80}-5′:GCAGAATTCGGCAACTGGAGAGAGCTAT; | (SEQ ID NO:92) |
| | | xis_{χ80}-3′:GCAGCTAGCAAGAGATCATCGGAGAG | (SEQ ID NO:93) |

^{a}Restriction sites are italicized.
Bases complementary to the template are underlined.

PCR fragments were generated by using as templates: pHH7013 and pHH7009 (obtained from J. C. Hu, Department of Biochemistry, TAMU, College Station, Tex.) for P_{syn1} and P_{syn4}; a derivative of pLD78 (Haldimann et al. (1998) J.Bacteriol. 180:1277-1286) called pLD81 (obtained from L. Daniels, Yale University, New Haven, Conn.) for P_{rhaS}; p34E-Tp (DeShazer et al. (1996) BioTechniques 20:762-764); obtained from D. E. Woods, University of Calgary Health Sciences Center, Calgary, Alberta, Canada) for dhfr; pBBR1MCS-5 (Kovach et al. (1995) Gene 166:175-176); obtained from M. Kovach, Louisiana State University Health Sciences Center, Shreveport, La.) for gen; λ (obtained from R. Somerville, Department of Biochemistry, Purdue University, West Lafayette, Ind.) for xis_{λ}; pBAD33 (Guzman et al. (1995) J.Bacteriol. 177:4121-4130); obtained from L.-M. Guzman, Department of Microbiology and Molecular Genetics, Harvard Medical School, Boston, Mass.); from pBS1attP6-1 (Campbell et al. (1992) Genetica 86:259-267); obtained from A. Campbell, Stanford University, Palo Alto, Calif.) for attP21, xis_{P21}, and int_{P21}; pEY109 (Yagil et al. (1989) J.Mol.Biol. 207:695-717); obtained from E. Yagil, Tel Aviv University, Israel) for attHK022, xis_{HK022}, and int_{HK022}; pJL10 and pJL110 (Leong et al. (1985) J.Biol.Chem. 260:4468-4477); from A. Landy) for attΦ80, xis_{Φ80}, and int_{Φ80} and attP22; P22 (obtained from S. Maloy, San Diego State University) for xis_{P22} and int_{P22}.

Due to the manner in which the CRIM plasmids (see FIG. 1) were constructed, the araBp region of pAH150, but not of pLA2, encodes an N-terminal portion of AraC as a fusion protein. As a consequence, araBp is expressed at a normal level in pLA2, but at a much reduced level in pAH150. All attP sites were designed taking into account information on DNA binding sites and structure (Leong et al. (1985) J.Biol.Chem. 260:4468-4477); (Wang et al. (1997) J.Bacteriol. 179:5705-5711); (Yagil et al. (1995) J.Mol.Biol. 252:163-177). Accordingly, the attP21 sequence encodes the C-terminus of icd and the attP22 sequence includes sequences for the thrW tRNA gene (Campbell et al. (1992) Genetica 86:259-267). CRIM plasmids with aadA, bla or gen facilitate certain constructions as they have a unique NcoI site.

Primers routinely used to verify cloned inserts by PCR or DNA sequencing include: rgnB-f (TTGTCGGTGAACGCTCTCCT (SEQ ID NO:49), ParaB-f (CACATTGATTATTTGCACGG (SEQ ID NO:94), PrhaB-f (CGTTCATCTTTCCCTGGT (SEQ ID NO:95)), and tL3-r (AGGATGCGTCATCGCCATTA (SEQ ID NO:96)). Priming sites rgnB-f and tL3 are common to all CRIM plasmids and are useful for sequencing inserts in all CRIM plasmids, except those containing lacI^{q}, in which tL3-r can be used.

Unexpectedly, we found that CRIM plasmids carrying attP22ₛ or attΦ80s (Table 3) failed to integrate or gave very few integrants, respectively, suggesting that additional att sequences are required (data not shown). Plasmids carrying a longer attΦ80 site (such as pAH153 and pAH162) integrate efficiently, while those carrying the longer attP22 site (such as pAH154) integrate less frequently than others.

All helper plasmids (see FIG. 2) express int alone or with xis from λp_{R} under cI857 control, which is also encoded by these plasmids, and are temperature-sensitive for plasmid replication. With one exception, the xis-int plasmids express these genes in the same orientation as found naturally. The construction of pAH129 resulted in placing xis_{Φ80} upstream of int_{Φ80} to create an xis-int_{Φ80} operon. We also deliberately destroyed the native BamHI site in int_{λ} during the construction of pAH57 by introducing a silent mutation with the xis_{λ}-5′ primer (Table 2).

CRIM plasmid integration. Cells carrying a CRIM helper plasmid were grown in 5 ml SOB cultures with ampicillin at 30° C. to an optical density at 600 nanometers (OD₆₀₀) of ca. 0.6 and then made electrocompetent. Following electroporation, cells were suspended in SOC without ampicillin, incubated at 37° C. for one hour, at 42° C. for 30 minutes and then spread onto selective agar and incubated at 37° C. Colonies were purified once non-selectively and then tested for antibiotic resistances for stable integration and loss of the helper plasmid, and by PCR for copy number (see below).

CRIM plasmid excision. Cells were transformed with the respective Xis/Int CRIM helper plasmid and then spread on ampicillin agar media at 30° C. Colonies were purified once or twice nonselectively on plates that were incubated for one hour at 42° C. and overnight at 37° C. They were then tested for antibiotic sensitivities and by PCR for loss of the integrated plasmid.

CRIM plasmid retrieval. P1kc lysates were made on integrants by using standard procedures. Recipient cells carrying the respective Xis/Int CRIM helper plasmid were grown in LB with ampicillin at 30° C. to early stationary phase and then infected with a P1kc lysate. Following phage absorption, centrifugation, and resuspension as described elsewhere (Wanner et al. (1994) In K. W. Adolph (ed.), Methods in Molecular Genetics, 3:291-310), the infected-cells were incubated for one hour at 37° C., 30 minutes at 42° C., an additional hour at 37° C., and then spread onto selective media (without ampicillin), for the CRIM plasmid, and incubated at 37° C. To recover plasmids by transformation, chromosomal DNAs were isolated from integrants, subjected to shearing by sonication or DNAse I digestion in the presence of divalent manganese (Anderson, (1981) Nucleic Acids Res., 9:3015-3027). Recipient cells carrying a helper plasmid were grown in 5 ml SOB cultures with ampicillin at 30° C. to an OD₆₀₀ of ca. 0.6 and then made electrocompetent. Following electroporation, cells were suspended in SOC without ampicillin, incubated at 37° C. for one hour, at 42° C. for 30 minutes and then spread onto selective agar and incubated at 37° C.

PCR verification of integrant copy number. Isolated colonies were picked with a plastic tip, suspended in 20 μl water. Five μl of the cell suspension, 10 pmol of each primer (P1 to P4 together), and 0.5 unit Taq DNA polymerase (New England Biolabs) were combined in 1× PCR buffer, 2.5 mM MgCl₂ and deoxynucleoside triphosphates in a final volume of 25 μl. PCR was carried out for 25 cycles of denaturing for 1 minute at 94° C., annealing for 1 minute (see Table 4), and extending for 1 minute at 72° C.

TABLE 4

PCR tests for integration of CRIM plasmids^{a}

Predicted sizes of PCR fragments
for attB with^{b}:

| | | | | | | Multiple |
|---|---|---|---|---|---|---|
| | | | | | | integrant |
| | | | T | None | Single integrant | P1/P2, P3/P2, |
| AttP | Primer P1 sequence | Primer P4 sequence | ° C. | P1/P4 | P1/P2, P3/P4 | P3/P4 |
| λ | GGCATCACGGCAATATAC | TCTGGTCTGGTAGCAATG | 63 | 741 | 577, 666 | 577, 502, 666 |
| | (SEQ ID NO:98) | (SEQ ID NO:99) | | | | |
| HK022 | GGAATCAATGCCTGAGTG | GGCATCAACAGCACATTC | 59 | 740 | 289, 824 | 289, 373, 824 |
| | (SEQ ID NO:100) | (SEQ ID NO:101) | | | | |
| χ80 | CTGCTTGTGGTGGTGAAT | TAAGGCAAGACGATCAGG | 63 | 546 | 409, 732 | 409, 595, 732 |
| | (SEQ ID NO:102) | (SEQ ID NO:103) | | | | |
| P21 | ATCGCCTGTATGAACCTG | TAGAACTACCACCTGACC | 57 | 506 | 568, 620 | 568, 682, 620 |
| | (SEQ ID NO:104) | (SEQ ID NO:105) | | | | |
| P22 | AAGTGGATCGGCATTGGT | TTCGTATCGACAGGAAGG | 63 | 735 | 376, 926 | 376, 568, 926 |
| | (SEQ ID NO:106) | (SEQ ID NO:107) | | | | |
| e14attR | CGCTTGAAGATGTGTGGT | GTTACGGTCTTGGCATTG | 57 | 862 | 1226, 389 | 1226, 682, 389 |
| | (SEQ ID NO:108) | (SEQ ID NO:109) | | | | |
| P22(EcoB) | AAGTGGATCGGCATTGGT | CGATTGAACCGCAGATTACG | 63 | 609 | 376, 801 | 376, 568, 801 |
| | (SEQ ID NO:110) | (SEQ ID NO:111) | | | | |

^{a}Primers P2 (ACTTAACGGCTGACATGG (SEQ ID NO:112)) and P3 (ACGAGTATCGAGATGGCA (SEQ ID NO:113)) are the same for all attP sites.
T, annealing temperature.
The P4 priming sites for attP21 and attP22 lie within prophage elements of wild-type E. coli K-12 (FIG. 2).
An att_{P21} CRIM plasmid integrants to the left (counterclockwise) of e14. The e14attR primers were used to test for integration to the right (clockwise) of e14. E. coli K-12, but not E. coli B, has an uncharacterized prophage in attP22.
An attP22 CRIM plasmid integrates adjacent (counterclockwise) to this prophage in E. coli K-12.
Primers for attP22(EcoB) were used to test for integration of attP22 CRIM plasmids in E. coli K-12 strains (BW25695 and others) with an “empty”attP22 site from E. coli B.
^{b}Sizes are in nucleotides.

DNA sequencing. The DNA sequences of all CRIM and CRIM helper plasmids were deduced in their entirety by verifying the sequence of all modules used in their constructions. PCR-amplified segments were verified by automated DNA sequencing of both strands after initial cloning. Many were initially cloned into SmaI-digested pSPORT1 (from Gibco BRL, Gaithersburg, Md.) or EcoRI and NcoI-digested pLITMUS29 (from New England Biolabs, Beverly, Mass.). Others were cloned directly into a CRIM plasmid and then sequenced. Several additional regions were also sequenced to permit generating detailed maps of all CRIM and CRIM helper plasmids.

Nucleotide sequence accession numbers. GenBank accession numbers for the CRIM plasmids are: AY048713 (pAH55; SEQ ID NO:1), AY048714 (pAH56; SEQ ID NO:2), AY048716 (pAH63; SEQ ID NO:3), AY048717 (pAH68; SEQ ID NO:4), AY048719 (pAH70; SEQ ID NO:5), AY048720 (pAH81; SEQ ID NO:6), AY048722 (pAH95; SEQ ID NO:7), AY048723 (pAH120; SEQ ID NO:8), AY054372 (pAH125; SEQ ID NO:9), AY048730 (pAH143; SEQ ID NO:10), AY048731 (pAH144; SEQ ID NO:11), AY048732 (pAH145; SEQ ID NO:12), AY048733 (pAH150; SEQ ID NO:13), AY048734 (pAH152; SEQ ID NO:14), AY048735 (pAH153; SEQ ID NO:15), AY048736 (pAH154; SEQ ID NO:16), AY048737 (pAH156; SEQ ID NO:17), AY048738 (pAH162; SEQ ID NO:18), AY048739 (pCAH56; SEQ ID NO:19), AY048740 (pCAH63; SEQ ID NO:20), and AY054373 (pLA2; SEQ ID NO:21). GenBank accession numbers for the CRIM helper plasmids are: AY048715 (pAH57; SEQ ID NO:22), AY048718 (pAH69; SEQ ID NO:23), AY048720 (pAH83; SEQ ID NO:24), AY048724 (pAH121; SEQ ID NO:25), AY048725 (pAH122; SEQ ID NO:26), AY048726 (pAH123; SEQ ID NO:27), AY048727 (pAH129; SEQ ID NO:28), AY048728 (pAH130; SEQ ID NO:29), AY048729 (pAH131; SEQ ID NO:30), and AY048741 (pINT-ts; SEQ ID NO:31).

Results and Discussion

General description. The basic CRIM plasmids are shown in FIG. 1. Each CRIM plasmid has four general regions in common: a polylinker or a cloning region consisting of a promoter for ectopic expression (the use of a non-native control sequence to regulate gene expression) with or without a regulatory gene; a phage attachment (attP) site; a conditional replication origin (oriR_{γ}); and a selectable marker. Several already contain an E. coli gene (lacZ, phoB, phoR, pstS, or uidAf) within the cloning region, however these genes are replaceable fragments that may be removed and replaced with a gene of interest in new constructions. In addition, all CRIM plasmids have bacterial (rgnB) and phage λ (t0, tL3) terminators flanking their cloning region to protect other segments from transcriptional read through. The CRIM plasmids were designed so that standard cloning methods can be used for making new ones with various combinations of these and other features, as necessary.

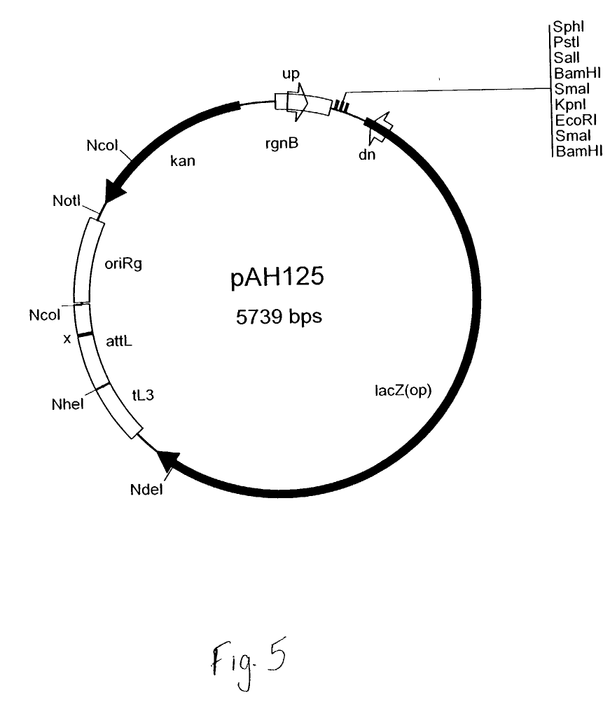

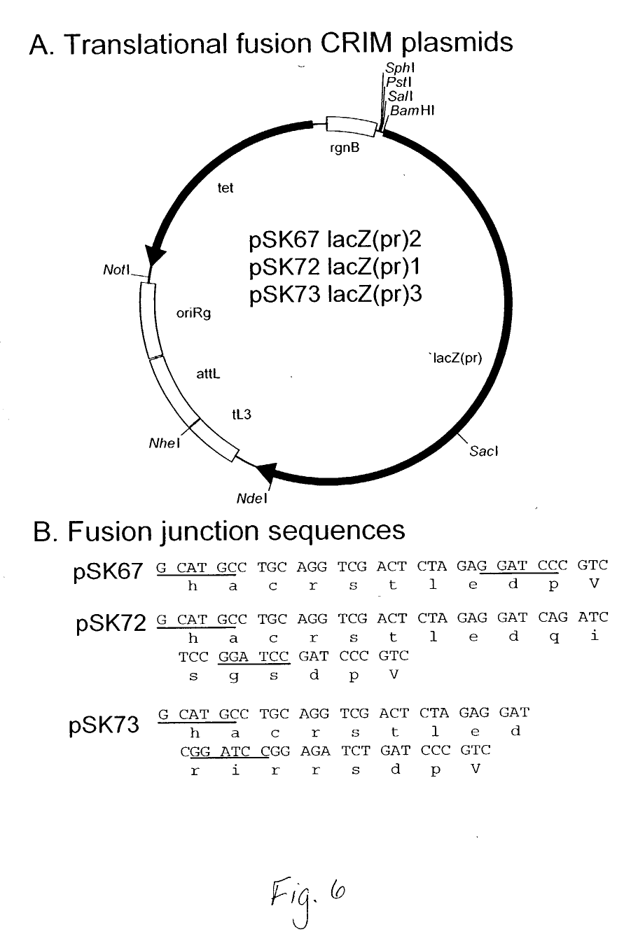

CRIM plasmid integration. The relative locations of the chromosomal attB sites in E. coli K-12 is shown in FIG. 3. Sites are based on the linkage map (Berlynet al. (1998) Microbiol.Mol.Biol.Rev. 62:814-984) and the position of the respective attB core sequences in the E. coli K-12 genome sequence (Blattner et al. (1997) Science 277:1453-1462) (for attλ: gCTTttTtatActAA (SEQ ID NO:114); attHK022: CTTTaggtgaa (SEQ ID NO:115); attP21: tGCtGCgcCATAT (SEQ ID NO:116); attP22: ATTcgtAATGcGAAG (SEQ ID NO:117); attΦ80: AACAcTTTcttAAAt (SEQ ID NO:118) (Campbell et al. (1992) Genetica 86:259-267); lowercase letters indicate bases that differ from the consensus (Boyd et al. (2000) J.Bacteriol. 182:842-847). E. coli K-12 has two attP22 sites separated by ca. 34-kb (at nucleotide 262125 and 296433 of the genome), which is consistent with their being separated by an uncharacterized phage. Furthermore, the use of PCR and P22(EcoB) primers (Table 2) indicates that the intervening sequences is absent from E. coli B, so it lacks this phage. Wild-type E. coli K-12 contains the prophage element e14 adjacent (clockwise) to attP21 (Campbell et al. (1992) Genetica 86:259-267).

CRIM plasmids can be simply integrated into the chromosome by direct transformation of normal (non-pir) hosts carrying a CRIM helper plasmid synthesizing the respective Int (FIG. 2, Table 2). Int synthesis from the helper plasmids is induced at elevated temperatures. Since the helper plasmids are also temperature-sensitive for replication (see Materials and Methods above), the resulting transformants are nearly always simultaneously cured of the helper plasmid. Upon integration at the respective attB site, all CRIM plasmids lie in the same relative orientation on the E. coli chromosome (FIG. 3). Therefore, even though they have sequences in common (tL3, oriR_{γ}, and rgnB), homologous recombination among them does not lead to instability because essential chromosomal genes lie between these attB sites. Due to the high efficiency of site-specific recombination, these homologies also do not interfere with integrating multiple CRIM plasmids at different attB sites in the same strain.

To test for single copy integration, a single PCR reaction with four primers (P1, P2, P3, and P4 in FIG. 4A; see Materials and Methods above) is routinely used. Single copy integrants are revealed as ones that have lost the fragment corresponding to the respective attB site (the P1 to P4 fragment) and simultaneously gained two new ones that are characteristic of the attL (BOP′; the P1 to P2 fragment) and attR (POB′; the P3 to P4 fragment) junctions.

Recombinants with two (or more) CRIM plasmids at the attB site are also easily distinguishable. Such multiple integrants gain also a third fragment characteristic of the attP site of the integrating plasmid (the P2 to P3 fragment). Recombinants resulting from integration elsewhere on the chromosome yield instead PCR products for both the respective attB (the P1 to P4 fragment) and attP (the P2 to P3 fragment) sites, provided that such integration occurs via homologous recombination or otherwise outside the attP region. Based on these criteria, we have shown that integration occurs primarily at the respective attB site and always requires the respective Int. The most common undesirable events are the occurrence of multiple copy integrants, however these seldom represent more than a few percent of the antibiotic-resistant transformants. With one exception, this is true for all CRIM plasmids.

The exception concerns the attP22 CRIM plasmids. These differ in two ways. First, the attP22 plasmids integrate ca. 100-fold less efficiently than the others. Second, one-half or more of the resulting attP22 plasmid integrants are often incorrect, and appear to occur via recombination events that do not involve the attP22 site. Since wild-type E. coli K-12 apparently has an uncharacterized prophage occupying the chromosomal attP22 site, we considered that this prophage interferes with site-specific integration at this site. However, we obtained similar results with an otherwise isogenic host lacking this prophage, suggesting that an additional factor or sequence is required for efficient attP22 recombination. Nevertheless, attP22 CRIM plasmids have still been quite useful for constructing strains that have multiple CRIM plasmids. In these cases, we have usually integrated attP22 CRIM plasmids before integrating others to prevent the attP22 plasmid from recombining with others via homologous recombination, which can also occur at low frequency. The attP22 CRIM plasmids may therefore be less valuable as vectors for library construction or other uses requiring high integration efficiency.

CRIM plasmid excision. Integrated CRIM plasmids can also be excised very efficiently. CRIM plasmid excision is carried out by using CRIM helper plasmids encoding both Xis and Int (FIG. 4B). We found all CRIM plasmids were easily eliminated from a specific attP site when using the respective Xis/Int helper plasmid, but not when using an Xis/Int helper plasmid for a different attP site. In most cases, 100% of the transformants were “cured” of the respective CRIM plasmid after a single colony purification step. No aberrant (non-specific) excision events were detected when using cells containing multiple CRIM plasmids integrated at different sites. We have used excision as a simple way to verify that novel phenotypes result from the presence of particular CRIM plasmids. For instance, in many cases additional experiments should be carried out to verify that a particular phenotype results from the presence of the plasmid. The ability to excise, that is, cure the strain of a CRIM plasmid provides an extremely efficient and useful means to eliminate that plasmid and therefore demonstrate that its presence (and not for example an unlinked mutation) is responsible for the phenotype. We have also found excision to be useful in certain strain constructions. For example, when studying complex metabolic or regulatory pathways, it has often been necessary to make strains containing multiple CRIM plasmids in various combinations. In such cases, it has occasionally been more convenient to excise a single CRIM plasmid from a strain containing a combination of different CRIM plasmids in order to introduce an alternative one than to construct an entirely new strain containing most of the same CRIM plasmids by integrating each individually.

CRIM plasmid retrieval. The ease of retrieving CRIM plasmids from the chromosome is an especially valuable attribute. Because Xis and Int catalyze excision and circularization of molecules from the respective alt sites, CRIM plasmids can be retrieved simply by introducing chromosomal DNAs from an integrant into permissive (pir⁺) hosts synthesizing Xis and Int from a helper plasmid. We have usually done this by using the generalized transducing phage P1kc, in a process that we have called PIX cloning (FIG. 4C; Haldimann et al. (1996) Proc.Natl.Acad.Sci.USA 93:14361-14366).

PIX cloning is done using recipients that are pir⁺, for replication of the CRIM plasmids, recA, to avoid homologous recombination events, and carry the respective Xis/Int CRIM helper plasmid. We measured the PIX cloning efficiencies by determining the number of antibiotic-resistant transductants per infectious phage in standard phage P1 crosses (Table 5). We assayed the transducing titer of the same P1kc lysates by determining the number of llv⁺ transductants. As shown in Table 5, PIX cloning is an extremely efficient process. Efficient retrieval occurs only in the presence of the respective CRIM helper plasmid. The recovered plasmids have always been correct, based on restriction enzyme analysis of plasmid DNAs isolated from several representative transductants in numerous such crosses. We have also used PIX cloning to recover plasmids for direct DNA sequence analysis (Haldimann et al. (1996) Proc.Natl.Acad.Sci.USA 93:14361-14366). In addition, we have shown that CRIM plasmids can be recovered following transformation of a recD pir⁺ host carrying the respective helper plasmids with chromosomal DNA (Materials and Methods). Accordingly, CRIM plasmids are also retrievable from bacteria that are insensitive to phage P1kc.

TABLE 5
PIX cloning efficiencies

| | Gm^{R} or Km^{R} |
|---|---|
| attP, CRIM plasmid | transductants per pfu^{a} |
| λ, pAH63 | 2.3 × 10⁻⁵ |
| HK022, pAH70 | 1.6 × 10⁻⁵ |
| Φ80, pAH153 | 1.9 × 10⁻⁴ |
| P21, pAH95 | 3.9 × 10⁻⁵ |
| P22, pAH154 | 1.8 × 10⁻⁷ |

^{a}Values are normalized to the Ilv⁺ transducing titers. The same P1kc lysates yielded ca. 10⁻⁴ to 10⁻³ Ilv⁺ transductants per plaque-forming units (pfu).

Using CRIM plasmids. Although CRIM plasmids can be used with most ordinary (non-pir) E. coli strains, we have made standard hosts for their use. These hosts have defined deletions of araBAD, rhaBAD, and lacZ, and are lacI^{q}. Hence, they cannot catabolize arabinose or rhamnose and yet encode the regulatory proteins (AraC, RhaR, and RhaS) required for ectopic expression of foreign genes from the respective (araBp, rhaBp, and rhaSp) promoters (also called P_{BAD} or P_{araB}, P_{rhaB}, and P_{rhaS}, respectively) These hosts provide tight regulation of these and LacI-controlled promoters. They can also be used with lacZ fusions generated using our standard CRIM lacZ transcriptional fusion vector pAH125 (FIG. 5). The lacZ, attP, oriRγ, and antibiotic resistance marker are modular to permit easy construction of new variants with different combinations of features. Expression levels of araBp in pLA2, rhaBp in pAH120, and rhaSp in pAH152 are similar to those reported elsewhere for the respective promoters (Haldimann et al. (1998) J.Bacteriol. 180:1277-1286). We have also shown elsewhere that rhaBp is an especially tightly regulated promoter (Haldimann et al. (1998) J.Bacteriol. 180:1277-1286). The synthetic (P_{syn1} and P_{syn4}) promoters provide for low-level unregulated gene expression. These promoters are juxtaposed to a ribosome binding site and AUG start codon that is contained within an NdeI site for convenient construction purposes. With the exception of plasmids carrying attP21, the NdeI site is unique (Haldimann et al. (1998). J.Bacteriol. 180:1277-1286).

Unexpectedly, we have recently found that araBp expression is much lower in pAH150 than in pLA2, which shows normal level expression (Haldimann et al. (1998) J.Bacteriol. 180:1277-1286). Lower expression in pAH150 results from interference by an N-terminal AraC′ fusion protein that is encoded by the araBp segment in pAH150, but not in pLA2. Nevertheless, both of these araBp CRIM plasmids have been useful as they both show arabinose-regulated promoter expression. pAH150 has been especially useful for conditional expression of regulatory genes, such as phoB, requiring low level expression, while pLA2 has been more useful for expression of structural genes requiring high level expression. Elsewhere, we have recently described E. coli hosts that show homogeneous expression of genes under araBp control, which constitutively synthesize the low-affinity AraE transporter from the chromosome.

We have also shown that attP22 and attλ CRIM plasmids integrate into the respective attB sites of Salmonella typhimurium. Since phages tend to exploit highly conserved and sometimes essential genes (e.g., tRNA genes) as sites for integration (Campbell (1992) J.Bacteriol. 174:7495-7499), several CRIM plasmids can probably integrate into chromosomes Of other bacteria, especially in other members of the family Enterobacteriaceae and related families. CRIM plasmids should therefore be useful in many applications involving bacteria other than common laboratory strains.

### Example 2

### Construction of Single-Copy lacZ Transcriptional and Translational Fusions Using CRIM Plasmids

Materials

Bacteria and plasmids. Strains are described in Table 6. A basic lacZ reporter CRIM plasmid is in FIG. 5. BW25141 (pir⁺) and BW25142 (pir-116) are used to propagate CRIM plasmids at medium (about 15 per cell) or high (about 250 per cell) copy number. CRIM helper plasmids for integration into and retrieval from attλ are in FIG. 8. All CRIM helper plasmids are listed in Table 7. Other lacZ reporter CRIM plasmids are shown in figures as cited herein. All CRIM helper plasmids are low-copy number plasmids that are temperature-sensitive for replication and hence easily curable. Most are similar to pINT-ts (or pAH57) and express int alone (or with xis) from λpᵣ under cI857 control, which is also borne by the plasmids. pKD15 expresses int behind araBp under AraC control, which is encoded by pKD15, while pKD16 expresses int behind UV5i under LacI control, which is encoded by pKD16. UV5i is a variant of lacUV5 with an idealized upstream operator.

TABLE 6
Bacterial strains

| Name^{a} | Description^{b} | Line^{c} | Source |
|---|---|---|---|
| BW22653 | lacI^{q} rrnB3 ΔlacZ4787 rph-1 | BD792 | Lessard et al. (1998) |
| | | | Chemistry & Biology 5: 489-504. |
| BW22831 | lacI^{q} Φ(ΔlacP rrnB3 araBp- | BD792 | Haldimann et al. J Bacteriol |
| | lacZ(op))1542 rpoS396(Am) | | 180: 1277-1286. |
| | DE(araBAD)567 rph-1 | | |
| BW25113 | lacI^{q} rrnB3 ΔlacZ4787 hsdR514 | BD792 | Haldimann et al. (2001) J |
| | DE(araBAD)567 | | Bacteriol 183: 6384-6393; |
| | DE(rhaBAD)568 rph-1 | | Lessard et al. (1998) |
| | | | Chemistry & Biology 5: 489-504; |
| | | | Datsenko et al. (2000) |
| | | | Proc Natl Acad Sci USA |
| | | | 97: 6640-6645; Khlebnikov et |
| | | | al. (2001) Microbiol 147: 3241-3247. |
| BW25141 | lacl^{q} rrnB3 ΔlacZ4787 | BD792 | Haldimann et al. (2001) J |
| | ΔphoBR580 hsdR514 | | Bacteriol 183: 6384-6393; |
| | DE(araBAD)567 | | Lessard et al. (1998) |
| | DE(rhaBAD)568 galU95 ΔendA9 | | Chemistry & Biology 5: 489-504; |
| | ΔuidA3::pir⁺ rph-1 recA1 | | Datsenko et al. (2000) |
| | | | Proc Natl Acad Sci USA |
| | | | 97: 6640-6645; Khlebnikov et |
| | | | al. (2001) Microbiol 147: 3241-3247. |
| BW25142 | lacI^{q} rrnB3 ΔlacZ4787 | BD792 | Haldimann et al. (2001) J |
| | ΔphoBR580 hsdR514 | | Bacteriol 183: 6384-6393. |
| | DE(araBAD)567 | | |
| | DE(rhaBAD)568 galU95 ΔendA9 | | |
| | ΔuidA4::pir-116 rph-1 recA1 | | |
| BW25993 | lacI^{q} hsdR514 DE(araBAD)567 | BD792 | Datsenko et al. (2000) Proc |
| | DE(rhaBAD)568 rph-1 | | Natl Acad Sci USA 97: 6640-6645. |
| BW28357 | lacI^{q} rrnB3 ΔlacZ4787 hsdR514 | BD792 | |
| | DE(araBAD)567 | | |
| | DE(rhaBAD)568 rph⁺ | | |
| BW29716 | lacI^{q} ΔlacZ1229::cat lacP−lacY | BD792 | |
| | hsdR514 DE(araBAD)567 | | |
| | DE(rhaBAD)568 rph⁺ | | |
| BW29717 | lacI^{q} ΔlacZ1230 lacP−lacY | BD792 | Cm^{S} of BW29716 with pCP20 |
| | hsdR514 DE(araBAD)567 | | |
| | DE(rhaBAD)568 rph⁺ | | |
| BW29718 | lacI^{q} ΔlacZ1229::cat lacP−lacY | MG1655 | |
| | rph⁺ | | |
| BW29720 | lacI^{q} ΔlacZ1230 lacP−lacY rph⁺ | MG1655 | Cm^{S} of BW29718 with pCP20 |
| BW29721 | lacI^{q} rrnB3 ΔlacZ4787 rph⁺ | MG1655 | |
| ^{a}All strains are λ⁻ and F⁻ derivatives of E. coli K-12. | | | |
| ^{b}All known mutations are indicated. The rph-1 and rph⁺ alleles refer to a frameshift mutation (Jensen (1993) J Bacteriol 175: 3401-3407) and the corrected allele, respectively. Strains are denoted as rph⁺ if they are descended from ones in which this mutation has been corrected. A mutant carrying the ΔlacZ1229::cat lacP−lacY fusion (SC824, obtained from S. Chiang, Harvard Medical School, Boston, MA) was made | | | |
| #by electroporation of BW25993 carrying pKD46 with a PCR product generated with pKD3 as template essentially as described elsewhere (Datsenko et al. (2000) Proc Natl Acad Sci USA 97: 6640-6645) by using PCR primers designed to remove lacZ entirely and to express lacY behind lacP under LacI control (obtained from S. Chiang). This mutation was then transferred by P1kc transduction to make BW29716 and BW29718, after which the FRT-flanked cat cassette was evicted | | | |
| #(as described by Datsenko and Wanner, 2000, Proc.Natl.Acad.Sci.USA 97: 6640-6645) | | | |
| #by using pCP20 (obtained from W. Wackernagel, Universitat Oldenburg, D-26111 Oldenburg, German) to generate the ΔlacZ1230 lacP−lacY fusion. | | | |
| ^{c}The line corresponds to the strain name when received from another lab. BD792 (CGSC6159) and MG1655 (CGSC6300) are in turn F⁻ derivatives of W1485F⁺. Both contain the rph-1 frameshift mutation that exists in many lines of “wild-type” E. coli K-12 (Jensen (1993) J Bacteriol 175: 3401-3407), which we corrected for those identified as being rph⁺. BD792 has the | | | |
| #rpoS396 (Am) mutation at the codon for Q33 of RpoS, which has been corrected in ancestors of all BD792 derivatives listed, except BW22831. | | | |

TABLE 7
CRIM helper plasmids

| attP site | Int helper plasmid^{a} | Xis/Int helper plasmid^{b} |
|---|---|---|
| attλ | pINT-ts, pKD15, pKD16 | pAH57 |
| attΦ80 | pAH123 | pAH129 |
| attHK022 | pAH69 | pAH83 |
| attP21 | pAH121 | pAH122 |
| attP22 | pAH130 | pAH131 |

^{a}pAH69, pAH121, pAH123, and pAH130 are similar to pINT-ts (FIG. 8), except each encodes the respective Int protein.
^{b}pAH83, pAH122, pAH129, and pAH131 are similar to pAH57 (FIG. 8), except each encodes the respective Xis and Int proteins.

Nucleotide sequence accession numbers. GenBank accession numbers for the CRIM plasmids are: AY054372 (pAH125; SEQ ID NO:9), AY054373 (pLA1; SEQ ID NO:32), and AY054373 (pLA2; SEQ ID NO:21). Other CRIM plasmids include pLA4 (SEQ ID NO:34), pLA5 (SEQ ID NO:35), pLA7 (SEQ ID NO:36), pLA8 (SEQ ID NO:37), pLA9 (SEQ ID NO:38), pLZ31 (SEQ ID NO:39), and pSK67 (SEQ ID NO:42). GenBank accession numbers for the CRIM helper plasmids are: AY048715 (pAH57; SEQ ID NO:22), AY048718 (pAH69; SEQ ID NO:23), AY048720 (pAH83; SEQ ID NO:24), AY048724 (pAH121; SEQ ID NO:25), AY048725 (pAH122; SEQ ID NO:26), AY048726 (pAH123; SEQ ID NO:27), AY048727 (pAH129; SEQ ID NO:28), AY048728 (pAH130; SEQ ID NO:29), AY048729 (pAH131; SEQ ID NO:30), and AY048741 (pINT-ts; SEQ ID NO:31). Other CRIM helper plasmids include pKD15 (SEQ ID NO:43), and pKD16 (SEQ ID NO:44). The ΔlacZ1230 lacP-lacY fusion junction is

(SEQ ID NO: 119)

GCGCAACGCAATTAATGTGAGTTAGCTCACTCATTAGGCACCCCAGGCTT
TACACTTTATGCTTCCGGCTCGTATGTTGTGTGGAATTGTGAGCGGATAA
CAATTTGTGTAGGCTGGAGCTGCTTCGAAGTTCCTATACTTTCTAGAGAA
TAGGAACTTCGGAATAGGAACTAAGGAGGATATTCAT.

Chemicals, media, buffers, and other supplies. 5-Bromo-4-chloro-3-indolyl-β-D-galactopyranoside (X-gal) is from Bachem (Torrance, Calif.), 1-S-octyl-β-D-thioglucoside (OTG) is from Anatrace (Maumee, Ohio), o-nitrophenyl β-D-galactopyranoside (oNPG), IPTG, L-arabinose, antibiotics, and ethylene glycol bis(β-aminoethyl ether) N,N,N′,N′-tetraacetic acid (EGTA, free acid) are from Sigma (St. Louis, Mo.). X-Gal is dissolved in dimethylformamide (DMF) at 40 mg/ml. When stored refrigerated in the dark, it is stable for about one month. A 1 M OTG stock is stored frozen (−20° C.), thawed and held on ice until use. A stock solution of 4% oNPG in water is stored frozen (−20° C.) indefinitely and used to make other oNPG solutions for assay. Other chemicals are from routine sources.

Luria-Bertani (LB) broth (without glucose), tryptone-yeast extract (TYE) agar (pH 7.0), M63, and MOPS media are prepared as described elsewhere (Wanner (1994) In K. W. Adolph (ed.), Methods in Molecular Genetics, 3:291-310). Media are solidified using Bacto agar (1.5% final). Nutrient broth (for freezing vials) contains 10 g Bacto Nutrient broth and 8 g NaCl per L. SOB (Hanahan D (1983) J Mol Biol 166:557-580) medium contains 2% tryptone, 0.5% yeast extract, 10 mM NaCl, 2.5 mM KCl, 10 mM MgCl₂, and 10 MM MgSO₄. It is prepared without Mg²⁺ and autoclaved. This is done by dissolving 20 g tryptone (Bacto), 5 g yeast extract (Bacto), and 0.5 g NaCl in ca. 950 ml water, adding 10 ml 0.25 M KCl, adjusting the pH to 7.0 with 1 N NaOH, adjusting the volume to 1 L and autoclaving. A 2 M Mg²⁺ stock solution (1 M MgCl₂.6H₂O and 1 M MgSO₄.7H₂O) is prepared and filter sterilized. To prepare SOB, 100 μl of the 2 M Mg²⁺ stock solution is added per 10 ml of the Mg²⁺-free SOB immediately before use. SOC medium is SOB with 20 mM D-glucose. SOC is made by adding 200 μl of filter-sterilized 1 M glucose per 10 ml SOB.

X-Gal is the blue dye for detection of β-galactosidase. Agar plates containing X-Gal are unstable and should be used within one week. When many X-Gal plates are prepared, the molten agar after autoclaving is cooled to 55° C., other ingredients are added, as necessary, and then 1 ml of the X-gal stock solution is added per liter. When only a few are required, 75 μl of the X-Gal stock solution is spread onto the surface with a glass or metal spreader and a turntable, or by using several (ca. 20 to 30) sterile 3 mm, soda lime glass beads and manually tilting the plate.

When following the growth of many cultures simultaneously, samples for cell density measurements are removed periodically and added to “fix” (0.5 ml formaldehyde per 100 ml water), to prevent further growth. When using a spectrophotometer, 0.5 ml samples are added to tubes containing 1.0 ml fix, the tubes are mixed by vortexing, and the diluted samples are later read. When using a microplate reader, 100 μl portions are pipetted directly into wells of a 96-well microplate containing 150 μl fix. Samples are mixed by pipetting and left at room temperature until they are read. Samples with an absorbance at 410 nanometers (A₄₁₀)>ca. 0.7 are further diluted to obtain accurate readings. All wells should contain the same total volume (250 μl) when readings are taken.

Z buffers for microtiter plate assays include: (a) Z stock buffer (pH 7.5), which contains 11.6 g anhydrous Na₂HPO₄, 2.48 g NaH₂PO₄.H₂O, 0.75 g KCl, and 0.246 g MgSO₄.7H₂O per liter and is stored refrigerated; (b) Z assay buffer, which contains also 1% β-mercaptoethanol (βME), 100 μg/ml chloramphenicol (Cm), and 15 mM OTG; and (c) Z holding buffer, which contains in addition 10% glycerol.

Microelectroporator chambers (for a BRL Cell-Porator) are from Labrepco (Horsham, Pa.). Standard 96-well flat-bottom polystyrene microtiter plates with covers (Evergreen scientific 290-8115-01F) and microplate sealing tape are from Life Science Products (Denver, Colo.).

Equipment. A variety of microplate readers, electroporators, and PCR machines are suitable. Protocols described in this chapter were carried out using a BRL Cell-Porator with Voltage Booster, Molecular Devices 340PC plate reader, and an MJ Research PTC-200 Cycler. Temperatures, times, and volumes may require adjustments with equipment of different models or manufacturers.

Methods

Bacteria preservation. Bacteria are routinely stored at −70° C. in nutrient broth or LB containing 8% dimethyl sulfoxide (DMSO). A one-half dram (2 ml) glass vial containing 1 ml nutrient broth (to which an antibiotic is freshly added for plasmid-bearing strains) is inoculated from a fresh isolated colony of a new strain. The vial is incubated at 30° C. on a roller until saturation (ca. 16 hours), then 90 μl DMSO (containing 0.5 ml 95% ethanol per 100 ml DMSO) is added, the vial is mixed briefly and then placed directly into the ultracold freezer.

Bacteria recovery. Bacteria are recovered without thawing by scraping the surface with a toothpick or pipet tip and colony-purified by streaking onto an appropriate agar medium (usually TYE agar without an antibiotic, except for plasmid-bearing strains). For physiology experiments, cells are adapted on agar of a similar composition prior to inoculation into liquid media. Cells are routinely passaged on M63 agar before growing them on MOPS agar. Typically, a strain is revived on TYE agar, isolated colonies are restreaked on glucose M63 agar and then on glucose MOPS agar before inoculation of glucose MOPS broth. Colonies from the glucose MOPS agar are streaked onto MOPS agar with different carbon sources prior to inoculation into the respective media. For inoculation, an isolated colony is suspended in ca. 50 μl of saline (0.85% NaCl) and ca. 10 μl portions are inoculated into culture tubes (or flasks) containing similar media without or with inducer. All strains are assayed in triplicate by suspending three colonies separately and inoculating one tube (or flask) without and with inducer with each suspension. 1 ml cultures are grown in 18 mm culture tubes with closures to permit air exchange in a tube roller. Larger volumes are grown in shake flasks.

Preparation of electrocompetent cells. To make electrocompetent cells, 1.5 ml culture is transferred to a microfuge tube, placed on ice for 2 minutes, and the cells are collected by centrifugation for 1 minute (at maximum speed in a high-speed microfuge at 4° C.). The supernatant is discarded and the cells are washed three times with 1 ml ice-cold 10% glycerol by resuspension and 15 second centrifugations. Care is taken to remove the supernatants promptly because the cell pellets are soft. After three washings, the cells are resuspended with 50 μl ice-cold 10% glycerol and held on ice until use. This yields a total of ca. 80 μl, or enough for four electroporations. The protocol is scaled up to prepare larger amounts of electrocompetent cells by using larger tubes and a high capacity centrifuge. Cells prepared in this way are routinely stored in 20 or 40 μl aliquots at 70° C. Frozen electrocompetent cells are thawed in an ice-water bath immediately before use.

Electroporation. For electroporation, 20 μl electrocompetent cells and 1 μl DNA (50 ng) are combined and placed into the electroporation cuvette. The Cell-Porator has a cuvette holding chamber that is filled with an ice-water mixture so the cells are held at 0° C. throughout the process. Electroporation is done as recommended by the manufacturer.

CRIM plasmid integration. Integration can be done by introducing the CRIM plasmid by using electroporation or chemically competent cells (Hanahan (1983) J Mol Biol 166:557-580). We nearly always use electroporation solely because it is more efficient.

Cells carrying the respective CRIM helper plasmid are prepared in advance by selecting ampicillin-resistant transformants at 30° C. For preparation of electrocompetent cells, a fresh isolated colony is inoculated into a small flask containing 5 ml SOB (with Mg²⁺ and ampicillin (100 μl/ml)) and incubated in a 30° C. shaking water bath. pINT-ts transformants are grown to an A₆₀₀ of ˜0.5-6 (ca. 5 to 6 hours) and the flask is then shifted to a 42° C. shaking water bath for ca. 20 minutes longer and then placed on ice. Growth in water baths is preferred to permit rapid temperature shifts. pKD15 and pKD16 transformants are grown to an A₆₀₀ of ˜0.2-3 (ca. 4 to 5 hours), then 1 mM IPTG (for pKD15) or 1 mM L-arabinose (for pKD16) is added, incubation is continued ca. 1 to 1½ hours longer to an A₆₀₀ of ˜0.6-7, and the flasks are placed on ice. Cells are then made electrocompetent as described above. Following electroporation, the cell-DNA mixture is added to 1 ml SOC (no antibiotic) in an 18 mm tube, incubated at 42° C. for 30 min., and then at 37° C. for 1 to 1½ hours longer. Portions are spread onto media that is selective for the CRIM plasmid, and lacking ampicillin to permit loss of the helper plasmid. Single-copy integrants of the lacZ CRIM plasmids described here are usually selected on TYE agar containing X-gal and kanamycin at 10 μg per ml or tetracycline at 8 μg per ml. Concentrations used with CRIM plasmids encoding different resistances are given elsewhere (Haldimann et al. (2001) J Bacteriol 183:6384-6393). After spreading, the plates are incubated 16 to 20 hours at 37° C. Cells are colony-purified without antibiotic selection before testing for loss of the helper plasmid (by testing for ampicillin sensitivity).

PCR lest of integrant copy number. Isolated colonies are picked up with a plastic tip or glass capillary, and suspended in 20 μl water. 5 μl of the cell suspension, 10 pmol of each primer (P1 to P4; FIG. 9), and 0.5 U of Taq DNA polymerase (New England Biolabs) are combined in 1× PCR buffer-2.5 mM MgCl₂ with deoxynucleoside triphosphates in a final volume of 25 μl. PCR is carried out for 25 cycles (denaturing for 1 min at 94° C., annealing for 1 min at 63° C. (for integration at attλ), and extending for 1 min at 72° C.). The same P2 and P3 primers are used with all CRIM plasmids, while the P1 and P4 primers are specific for the chromosomal attB site (FIG. 9). Single PCR reactions are run with all four primers in the same tube. Different annealing temperatures and P1 and P4 primers are used for CRIM plasmids that integrate at different attB sites. Usually three or four colonies are picked directly from the selection plate and initially tested by PCR for single-copy ones. Two or three single-copy candidates are then colony-purified nonselectively once or twice and retested by PCR to be sure that they are pure and stable.

As depicted in FIG. 9, CRIM plasmids are integrated into a normal (non-pir) host under conditions of Int synthesis. Integration of an attλ CRIM plasmid occurs by site-specific recombination at attB, which lies between the chromosomal gal and bio loci. Integrates are selectable as antibiotic-resistant transformants because the CRIM plasmids cannot replicate in the absence of Π. When using the P1 to P4 primers, a control strain with an empty attB site yields a single attB (741-nt, P1/P4) PCR product and those with a single integrated CRIM plasmid yield both attL (577-nt, P1/P2) and attR (666-nt, P3/P4) PCR products. Ones with multiple CRIM plasmids integrated in tandem yield three products: (i) an attL (577-nt, P1/P2) PCR product, (ii) an attR (666-nt, P3/P4) PCR product, and (iii) a CRIM plasmid-specific (502-nt, P2/P3) PCR product. Integration can also occur by homologous recombination, although this is usually infrequent. These recombinants are recognizable as ones with an attλ (741-nt, P1/P4) and a CRIM plasmid-specific (502-nt, P2/P3) PCR products. The latter is seen because the attλ region of the CRIM plasmid is uninterrupted in such recombinants. Primer sequences are: P1, GGCATCACGGCAATATAC (SEQ ID NO:98); P2, ACTTAACGGCTGACATGG (SEQ ID NO:112); P3, ACGAGTATCGAGATGGCA (SEQ ID NO:113); and P4, TCTGGTCTGGTAGCAATG (SEQ ID NO:99).

CRIM plasmid retrieval. Retrieval is carried out by transduction or transformation of a recipient that is pir⁺, for replication of the CRIM plasmids, recA, to avoid homologous recombination events, and carrying the respective Xis/Int CRIM helper plasmid. Accordingly, the CRIM plasmid enters the recipient as a linear DNA molecule attached to chromosomal DNA of the donor. Upon entry into the recipient, X is and Int catalyze the excision and recircularization of the CRIM plasmid, which then replicates as a free plasmid. In essence, site-specific recombination results in cloning the CRIM plasmid from the chromosome. Because we usually do this by using the generalized transducing phage P1kc, we have termed the process PIX cloning for P1, Int, Xis cloning (Haldimann et al. (1996) Proc Natl Acad Sci USA 93:14361-14366).

P1kc lysates are made on the integrants by standard procedures (Wanner (1994) Methods in Molecular Genetics, 3:291-310). Recipient cells are grown in 1 ml LB with ampicillin at 30° C. to early stationary phase in 18 mm tubes (in a roller). Cells are collected by centrifugation, resuspended in 10 mM MgCl₂, 2.5 mM CaCl₂. A portion of the lysate (usually 5 μl) is added to 100 μl cell suspension, and the mixture held at room temperature. After 20 min., 1 ml LB containing 10 mM EGTA (neutralized with NaOH) is added, the tube is vortexed, and the cells are collected by centrifugation. Cells are resuspended in 1 ml LB with 10 mM EGTA (no antibiotic), incubated at 37° C. for one hour, at 42° C. for 30 min, and at 37° C. for an additional hour. Portions are then spread onto TYE agar with an antibiotic to select for the CRIM plasmid (without ampicillin) and incubated at 37° C. The protocol and strains used for the recovery of CRIM plasmids by DNA transformation are described elsewhere (Haldimann et al. (2001) J Bacteriol 183:6384-6393).

Simple plate test for estimation of β-galactosidase activity. X-Gal is a very sensitive indicator of β-galactosidase activity, however it is not very quantitative. To estimate relative β-galactosidase levels among different strains, we use oNPG instead. In the past, we did this dripping a solution of 0.4% oNPG in Z buffer containing 0.05% SDS (without βME or Cm) onto colonies grown on an agar medium (Agrawal et al. (1990) J Bacteriol 172:3180-3190). Since OTG is a more effective lysing agent than SDS under these conditions, we now routinely do this by using instead a solution of 0.4% oNPG in Z buffer containing 15 mM OTG. For comparisons, appropriate negative (Δlac) and positive (Lac⁺ constitutive) control strains are tested side-by-side. The 0.4% oNPG Z buffer solution is stored refrigerated for up to one week.

Cell growth and microplate β-galactosidase assays. Our standard protocols for measuring cell growth and β-galactosidase activities when using test tubes and a spectrophotometer are described in detail elsewhere (Wanner (1994) Methods in Molecular Genetics, 3:291-310). A microplate reader facilitates assaying many more samples and the use of smaller volumes. While standard spectrophotometric and microplate β-galactosidase assays are basically similar, a few differences are notable. For tube assays, cells are usually lysed by treatment with SDS and chloroform. We use instead 15 mM OTG to lyse cells for microplate assays because it is rapid and more effective than other lysis methods. Tube assays are usually stopped before reading by adding ca. one-half volume of 1 M Na₂CO₃, which raises the pH to ca. 10. Since tubes with more enzyme turn yellow more rapidly, they are stopped earlier and the times are noted. For simplicity, microplates are instead read continuously without stopping the reactions until all samples turn sufficiently yellow. Although all data are collected automatically, we use only those absorbance readings within a set range (ca. 0.05 to 0.7) to calculate the results.

It should be noted that the absorbance of o-nitrophenol changes with pH. There is an ca. 2.5-fold increase from pH of 6.9 to 8.6, while β-galactosidase activity is only modestly affected between pH 6.9 and 7.6. Therefore, to enhance color development of o-nitrophenol, we use Z buffers at pH 7.5 (instead of pH 7.0) for microplate β-galactosidase assays. It is also important that all wells contain the same total volume. We run assays with a total volume of 250 μl per well. Cross contamination can also be a serious problem if assays are run for long periods (>ca. 2 hours). For short periods, plates are covered with standard lids between readings. For samples requiring long incubations, the wells are sealed with microplate sealing tape in order to prevent cross contamination due to vaporization and condensation of o-nitrophenol. Our detailed protocol follows:

Step 1. Cultures are sampled by removing 100 μl portions. For absorbance readings, 100 μl samples are added directly to 150 μl fix in wells of a microplate and mixed by pipetting. For enzyme assay, a second 100 μl sample is added to a microfuge tube containing 200 μl Z assay buffer and the tube is vortexed. Accordingly, the dilution factors for the cell density measurements and β-galactosidase assays are 2.5 and 3.0, respectively (step 9). The microplate is left at room temperature. The enzyme samples are kept in a floating microfuge rack in an ice-water bath (0° C.) till all data are collected and evaluated (step 7). If these samples are to be kept more than several hours before they are assayed, then Z holding buffer is used instead.

Step 2. The absorbance values of the “fixed samples” are measured in a plate reader at A₄₁₀. If values exceed ca. 0.7, then further dilutions are made as necessary. The dilution factor is corrected accordingly. For convenience, the cell densities and β-galactosidase assays (o-nitrophenol production) are both read at A₄₁₀.

Step 3. To measure β-galactosidase, 30 μl portions of the enzyme samples are added to microplate wells containing 200 μl Z assay buffer. Extreme care should be taken to avoid air bubbles, which can affect the readings. Once substrate is added (step 6), the total assay volume is 250 μl, so the sample dilution factor is 8.33. Depending upon the cell density and amount of enzyme, the portion assayed is varied. For cultures with an actual A₄₁₀ between 0.2 and 1.5, 30 μl portions are usually adequate. The amounts are adjusted as necessary so the cell absorbance in the wells is ca. 0.02 or less. Even though OTG treatment rapidly permeabilizes cells to oNPG and reduces their turbidity, the cell densities continue to decrease for many hours. For initial values <0.02, these changes are negligible even after 16 hour incubations. We also vary the portions assayed so that the assay time is at least 10 minutes and generally less than 16 hours (step 7). If portions are varied, the amounts of Z assay buffer are changed to maintain a total assay volume after substrate addition of 250 μl.

Step 4. The microplate samples are read to obtain an initial value of the OTG-treated cells. Due to the OTG treatment, these values are less than the values of the fixed samples, even after correcting for dilution. The actual differences vary and depend on the time of OTG treatment, culture condition, and strain. This is why we use an initial cell density of the OTG-treated cells <0.02. If samples with higher cell densities are assayed, then additional controls (OTG-treated cells without substrate) are run in parallel for each sample.

Step 5. The microplate with the OTG-treated cells is incubated at 28° C. for 10 minutes. During this time, the oNPG substrate solution is also prewarmed to 28° C. With a temperature-controlled reader and a single microplate, the reader itself can be used for incubation. When assaying several microplates, they are incubated on a metal surface inside a constant temperature incubator nearby.

Step 6. The assay is started by adding 20 μl 0.4% oNPG to each well with a multichannel pipettor. When doing kinetic assays, the actual times of the first (t₀, time zero) and last (tₑ, time end) readings are arbitrary.

Step 7. Plates are read at 5, 10, and 20 minutes and at later times until all samples reach an A₄₁₀ of 0.2 to 0.6 or 16 hours, whichever occurs sooner. If samples turn yellow too quickly, e.g., reach an A₄₁₀>0.8 in less than 10 minutes, they are re-assayed by using smaller portions of the enzyme samples.

Step 8. Blanks containing Z assay buffer with oNPG but no cells are always run. As mentioned in step 4, controls for OTG-treated cells (without substrate) are sometimes also necessary.

Step 9. Enzyme units are calculated in terms of activity (nanomoles per min) per ml of culture and specific activity (activity per cell density or mg protein) using the molar extinction coefficient, ε₄₁₀, for o-nitrophenol of 4,500. After subtraction of the blank, results are calculated using the following equations:

Activity (nmoles/ml/min)=3.0(CDF)·[A₄₁₀(tₑ)−A₄₁₀(t₀)]·8.33(SDF)·222(CF)/(tₑ−t₀)

where CDF is the culture dilution factor (3.0 in step 1), SDF is the sample dilution factor (8.33 in step 3), and CF is the conversion factor (222 or 10⁶/4,500, for conversion of absorbance to nanomoles per ml). A₄₁₀(tₑ) and A₄₁₀(t₀) are the absorbances at the end (tₑ) and start (t₀, zero time), respectively. The actual values of CDF and SDF vary depending upon the dilutions when sampling the cultures and the portions assayed.

Specific activity=Activity/[2.5(FDF)·A₄₁₀(cells)]

where FDF is the fix dilution factor (2.5 in step 1), and A₄₁₀ (cells) is the cell density in step 2. If different volumes are taken for the cell density and enzyme samples, then the appropriate values are used in the calculations.

Troubleshooting

Problems can arise during the construction of specific lacZ fusion CRIM plasmids or upon integrating them into the chromosome. While it is desirable to use high-copy-number (pir-116) E. coli hosts (Metcalf et al., (1994) Gene 138:1-7) for ease of plasmid preparation, plasmids carrying strong promoters or particular genes can be deleterious in these hosts. We have on occasion found both promoter and lacZ mutations in CRIM plasmids isolated from such high-copy-number hosts. We therefore routinely use medium-copy-number (pir⁺) hosts to prepare CRIM plasmid DNAs when characterizing promoters of unknown strength. We use high-copy-number (pir-116) hosts only to prepare CRIM plasmid DNAs when examining weak or tightly controlled promoters.

Following electroporation and selection of integrants, the helper plasmid is usually lost because it is unstable at 37° C. and ampicillin is omitted. If it is not lost, then a few colonies are colony-purified nonselectively once at 43° C. and retested. Once integrants are chosen from the initial selective media, we routinely propagate them in the absence of an antibiotic. Since CRIM plasmids integrate by site-specific recombination, the integrants are quite stable. It is also preferable not to maintain them on an antibiotic medium to prevent inadvertent selection of multiple copy integrants, which can also occur subsequently.

We routinely test about three colonies by PCR as described above to find single-copy integrants, which are usually the majority. The most frequent undesirable event is the formation of multicopy (tandem) integrants at the respective attB site. These can predominate if too high an antibiotic concentration is used in the selective medium. We have used the concentrations described above in CRIM plasmid integration to isolate single-copy integrants of hundreds of E. coli K-12 and a few Salmonella typhimurium strains. Occasionally, we varied these concentrations to reduce background growth or to find single-copy integrants.

A second undesirable event can occur if the CRIM plasmid recombines elsewhere on the chromosome, and not at the respective attB site. These are also recognizable by the standard PCR test (FIG. 9). They probably result from homologous recombination of the CRIM plasmid with the bacterial chromosome. For example, a CRIM plasmid carrying an E. coli promoter-lacZ fusion can recombine via promoter sequences in common with the chromosome. We also frequently integrate CRIM plasmids into cells containing an integrated CRIM plasmid at a different attB site(s). In such cases, homologous recombination can occur with the resident plasmid because all CRIM plasmids have sequences in common (tL3, oriRγ, and rgnB). Importantly, these events are seldom problematic due to the high efficiency of site-specific recombination. When they occur, they are attributable to inadequate Int synthesis. The simplest remedy is to repeat the integration with newly prepared electrocompetent cells. Sometimes, a new transformant carrying the CRIM helper plasmid is used. Changing the induction protocol can also help. A CRIM helper plasmid that synthesizes Int under a different control (e.g., induction by temperature-shift or by arabinose or IPTG addition; FIG. 8) can also be advantageous.

Applications

Reporter CRIM plasmids are useful both for studying specific regulatory regions and for screening random fusion libraries to identify new promoters. Recombinants carrying specific or random promoter-lacZ fusions are generated using standard molecular biological methods (Ausubel et al. (2002) Current Protocols in Molecular Biology. John Wiley & Sons, New York). To construct specific fusions, promoter fragments are conveniently synthesized by PCR using primers with 5′ extensions containing restriction sites for directional cloning into a transcriptional fusion CRIM plasmid such as pAH125 (FIG. 5). Translational fusions are similarly made by using a translational fusion CRIM plasmid such as pSK67 (FIG. 6).

These plasmids can also be used for the construction of random lacZ fusion libraries. For example, we have used equimixtures of the translational fusion CRIM plasmids pSK67, pSK72, and pSK73 (digested with BamHI) to prepare libraries with E. coli chromosomal DNA (digested with Sau3A) to search for genes controlled by the response regulator CreB. To do this, we integrated the libraries directly into the chromosome of a ΔlacZ ΔcreB host carrying a tightly regulated, rhamnose inducible rhaBp-creB⁺ fusion elsewhere on the chromosome (Haldimann et al. (1998) J Bacteriol 180:1277-1286). By screening ca. 6,000 colonies that were blue (Lac⁺) on X-Gal agar in the presence of rhamnose, we found thirteen that were less blue in its absence. We retrieved these CRIM plasmids, sequenced their inserts, and then integrated them into the same and different hosts to study their regulation. As expected, a few contained the rhamnose-regulated rhaS and rhaT promoters. Several others contained regions upstream of yidS and yieI, suggesting that they are targets of CreB. While the goal was to unmask the role of CreB by identifying its gene targets, this was not accomplished because no role can be ascribed to YidS, YieI, or other co-transcribed genes. Nevertheless, these experiments established the utility of lacZ reporter CRIM plasmids for the preparation and screening of random lacZ fusion libraries in single-copy.

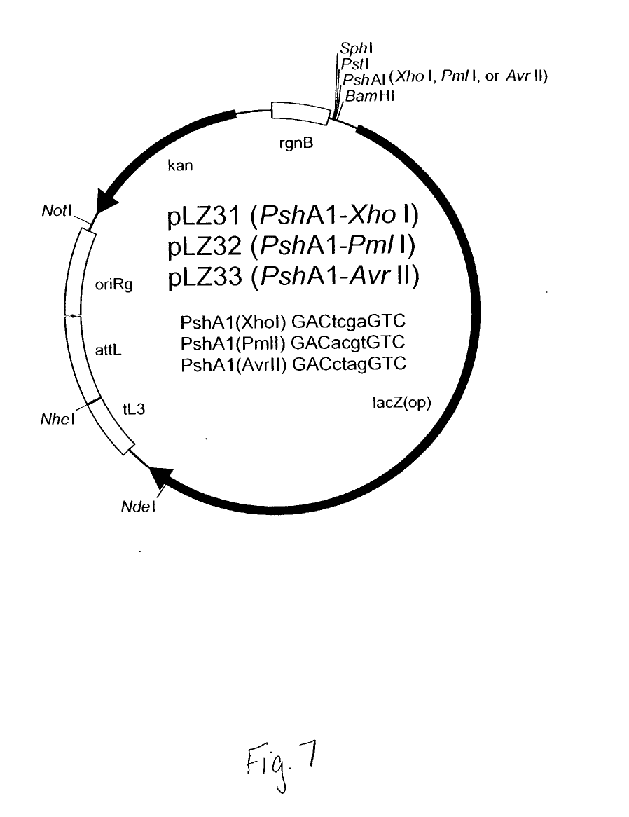

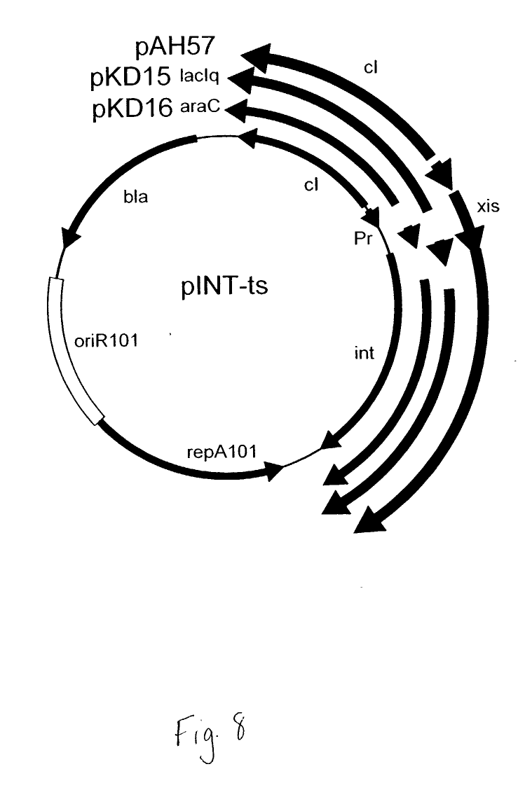

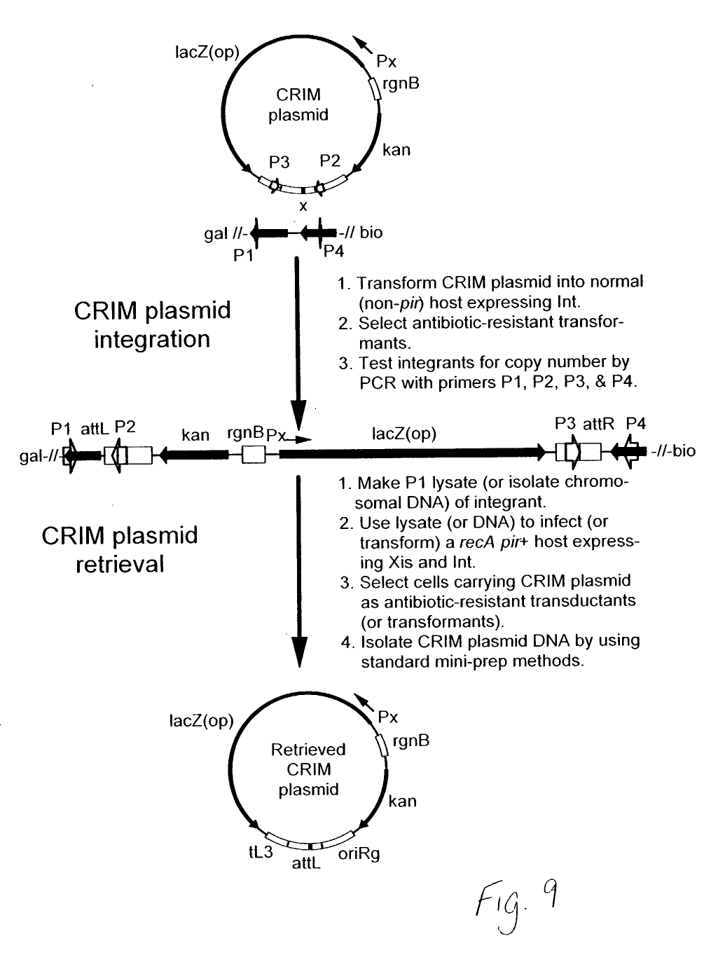

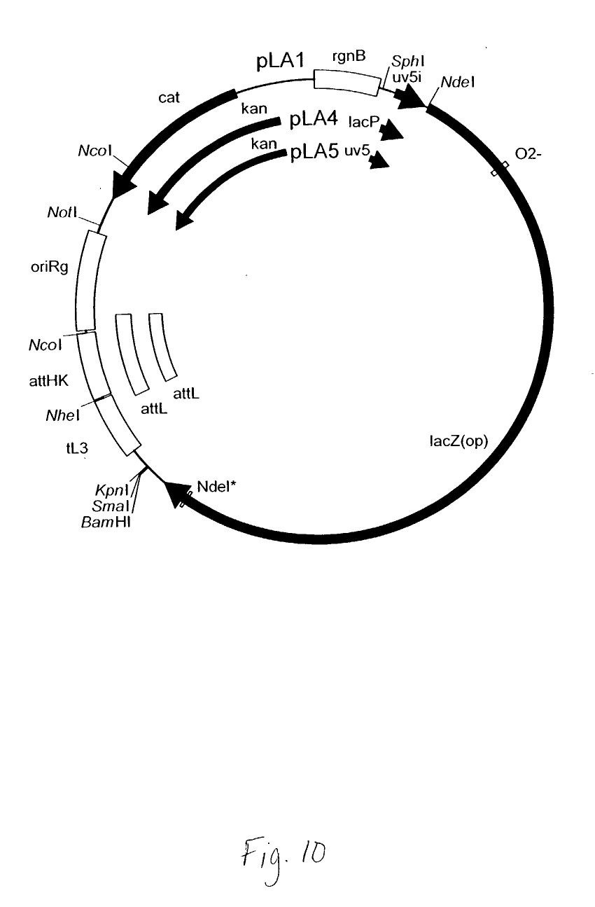

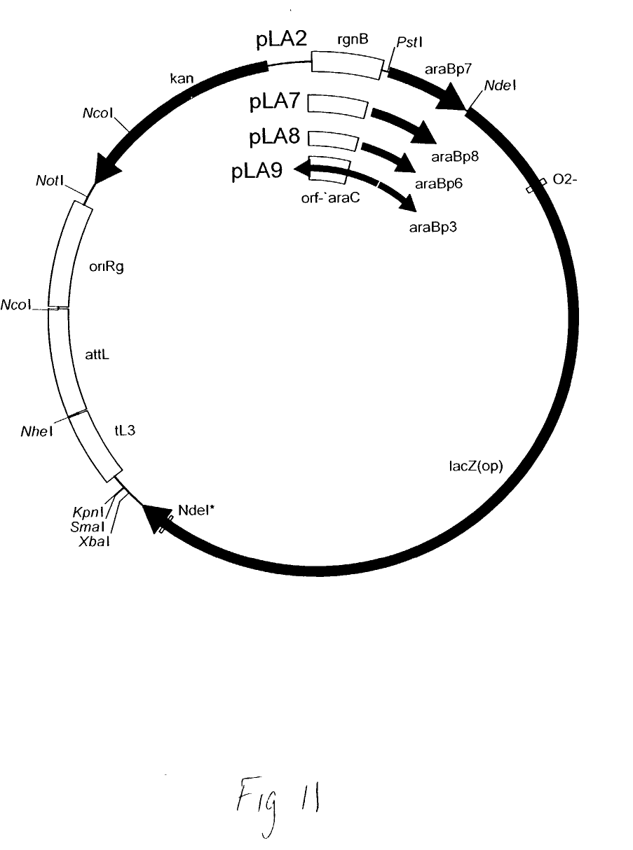

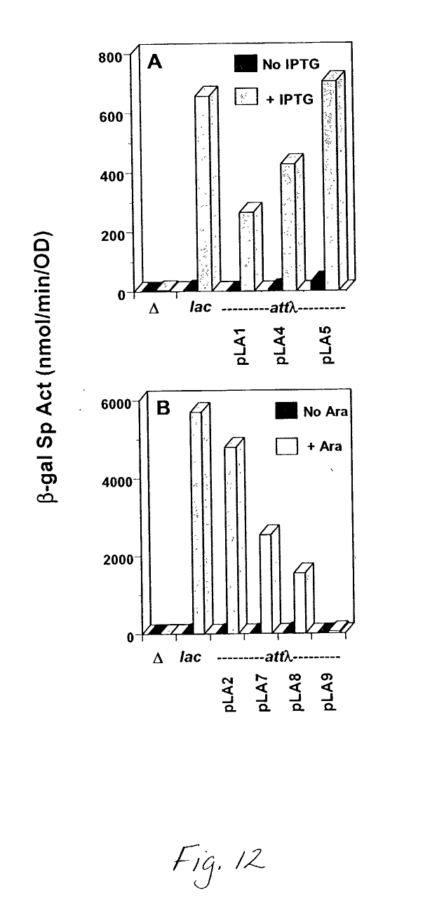

One possible limitation of screening random fusions is the large numbers of integrants that should be tested. For example, to find all promoters in an ca. 5-megabase genome at a 95% confidence limit would require testing over 50,000 integrants, or at 99% confidence limits over 250,000 integrants. These values were calculated based on the assumptions that the libraries contain random ca. 500-bp fragments and the promoter/regulatory regions are distributed randomly around the chromosome within ca. 250-bp segments. Further, this number would even be larger if the libraries are “contaminated” with plasmids without inserts. To overcome this problem, we recently made new lacZ reporter CRIM plasmids (FIG. 7), which are based on zero-background cloning vectors developed elsewhere (Majumder et al. (1994) Gene 151:147-151).

Conditional expression is a powerful genetic tool. It was first used to show that a control region is separable from its structural gene in the analysis of a fusion that expressed LacY under purine control (Jacob et al. (1965) J Mol Biol 31:704-719). Shortly afterwards conditional (adenine-dependent) expression of this fusion was used to clone the first gene (lacz) into a phage vector by directed transposition (Beckwith et al. (1966) J Mol Biol 19:254-265). This approach has been used innumerable times since, for example, to show that the first trp-lacZ, ara-lacZ, and phoA-lacZ fusions were fused to the respective promoters (Casadaban et al. (1975) Proc Natl Acad Sci USA 72:809-813); Reznikoffet al.(1969) J Mol Biol 43:201-213); (Sarthy et al. (1979) J Bacteriol 139:932-939). Likewise, searches for damage-inducible (din), phosphate-starvation-inducible (psi), and in vivo inducible (ivi) genes (Kenyon et al. (1980) Proc Natl Acad Sci USA 77:2819-2823); (Wanner et al. (1981) J Bacteriol 146:93-101); (Mahan et al. (1993) Science 259:686-688) were logical extensions of this approach. Accordingly, many methods have been since developed that exploit conditional expression to study cell biology (Brown (1987) Cell 49:825-833); Guzman et al.(1995) J Bacteriol 177:4121-4130).

Our basic CRIM plasmids include ones with promoters controlled by IPTG, arabinose, or rhamnose (Haldimann et al.(2001) J Bacteriol 183:6384-6393). To assess their regulation, representative promoters controlled by IPTG and arabinose were cloned into an appropriate lacZ reporter CRIM plasmid (FIG. 10 and FIG. 11). Unlike the CRIM plasmids in FIGS. 5, 6, and 7, pLA1, pLA4, and pLA5 (see FIG. 10) are derivatives of a lacZ CRIM plasmid in which the native lacO2 operator was changed to the O2− sequence (\ddot{M}uller et al. (1996) J Mol Biol 257:21-29), the native NdeI site in lacZ was eliminated by a silent mutation, and a unique NdeI site was introduced adjacent to the Met start codon of LacZ (Haldimann et al. (2001) J Bacteriol 183:6384-6393), pLA1 has an idealized upstream operator and the native lacO1 downstream of lacUV5, hence the designation UV5i. In pLA4 and pLA5, lacP and lacUV5 originated as PCR fragments using as templates pUC19 (Yanisch-Perron et al., (1985) Gene 33:103-119), and pRZ6522 (Noel et al. (2000) J Biol Chem 275:7708-7712); obtained from W. Reznikoff, University of Wisconsin, Madison, Wis.), respectively. The entire lacZ with the modified lacO2 region and NdeI sites and all promoter segments were sequenced after initial cloning. Due to how they were constructed, the native EcoRI site of lacZ is also absent in these plasmids. O2− and NdeI* mark the locations of the respective mutations.

pLA2, pLA7, pLA8, and pLA9 (see FIG. 11) have the identical lacZ region as those shown in FIG. 10. The araBp segments are denoted with alleles to indicate their origin and different sequences. The araBp3 (in pLA9) has the same region as pAH 150 (Haldimann et al. (2001) J Bacteriol 183:6384-6393), which exhibits arabinose-dependent PhoB synthesis (Haldimann et al. (1998) J Bacteriol 180:1277-1286). The araBp6 (in pLA8) was generated by PCR with pAH150 as template and cloned as a 152-nt araBp fragment lacking native araBpO2 (Dunn et al. (1984) Proc Natl Acad Sci USA 81:5017-5020). The araBp7 (in pLA2) was generated by PCR with pBAD33 (Guzman et al. (1995). J Bacteriol 177:4121-4130) as template; it has the entire araBp regulatory region of E. coli B. The araBp8 (in pLA7) was generated by PCR with strain MS 1868 (obtained from from P. Youderian, University of Idaho, MOscow Idaho (at time of receipt; now at TAMU, College Station, Tex.) as template; it has the entire araBp regulatory region of S. typhimurium LT2. As described herein, all of these promoters, except araBp3, express comparable levels of β-galactosidase. An examination of the sequences revealed that pLA9 (like pAH31 and pAH150) is predicted to encode 55 N-terminal residues of AraC. Furthermore, this araC′ segment is joined to plasmid sequences such that pLA9 and pAH150, but not pAH31, encode a 246-residue araC′-orf fusion protein due to translational read through of the rgnB region. In contrast, pAH31 is predicted to encode an AraC′ fusion protein with only a 10-residue C-terminal extension. The introduction of stop codons preceding rgnB restores high level expression in a derivative with the same araBp3 region, thus down regulation of araBp in pLA9 (and pAH 150) is a consequence of expressing this AraC′-orf fusion protein. All segments generated by PCR were sequenced after initial cloning.

In general, the expression of these promoters depicted in FIGS. 10 and 11 was induced by IPTG or arabinose and the induced levels were similar (FIG. 12). The strains in panel A of FIG. 12 were assayed following 16 hours growth in 0.06% glucose MOPS without or with 1 mM IPTG, and the strains in panel B were assayed following 16 hours growth in 0.1% glycerol MOPS without or with 1 mM L-arabinose. In panel B, the BW22831 (araBp3-lacZ⁺) strain was created by recombining the araBp3 fusion from pAH31 onto the chromosome by homologous recombination in place of lacP (Haldimann et al. (1998) J Bacteriol 180:1277-1286). Under these conditions, the lacUV5 promoter is the highest among the IPTG-regulated promoters. With one notable exception, the arabinose-regulated promoters are induced to even higher levels. The exception concerns araBp3 in pLA9. The araBp3-lacZ fusion in this CRIM plasmid was unexpectedly found to express lacZ at only ca. 2% of the level of the identical fusion when recombined onto the chromosome in front of the lac operon (FIG. 12; (Haldimann et al. (1998) J Bacteriol 180:1277-1286). Yet, it is highly inducible (ca. 40-fold) by arabinose, in agreement with studies showing that a similar CRIM plasmid with an araBp3-phoB⁺ fusion displays an arabinose-dependent PhoB phenotype (Haldimann et al. (1998) J Bacteriol 180:1277-1286). The low expression level of araBp3 in CRIM plasmids is attributable to the fortuitous creation of an artificial AraC′ fusion protein that interferes with normal inducibility.

Several reporter systems are now available for constructing fusions. Major advantages of lacZ fusions are the availability of alternative substrates, ease of assay, and perhaps most of all genetic selections (Beckwith (1970) In J. R. Beckwith and D. Zipser (eds.), The Lactose Operon). X-gal is clearly the most sensitive and widely used substrate for detecting lacZ activity. X-gal itself is colorless. Upon cleavage of X-gal by β-galactosidase, an indigogenic product is formed that is spontaneously oxidized in air to an insoluble blue pigment (Horwitz et al. (1966) J Med Chem 9:447). Because its uptake does not require LacY, X-gal is an especially useful indicator of β-galactosidase in most cells. However, its sensitivity also makes it less useful in genetic selections that require differentiating mutants with elevated or decreased lacZ expression levels.

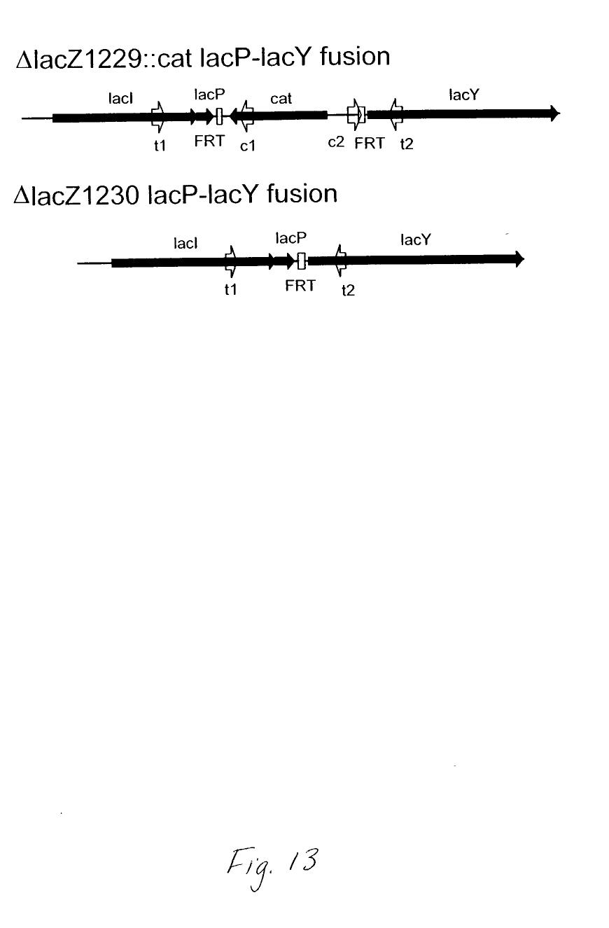

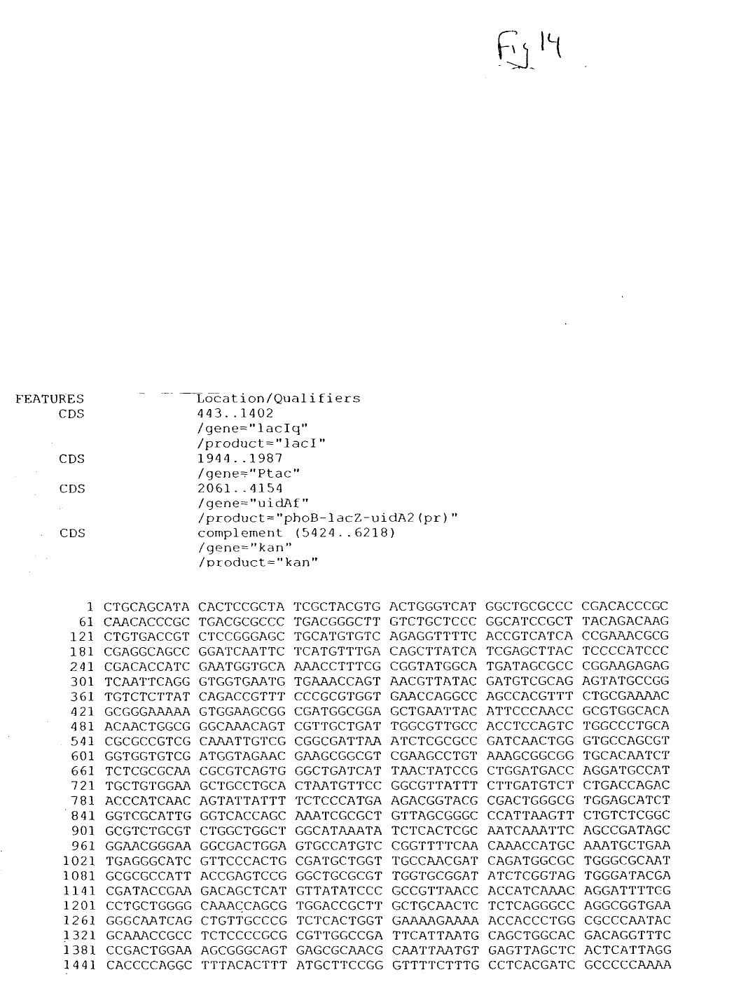

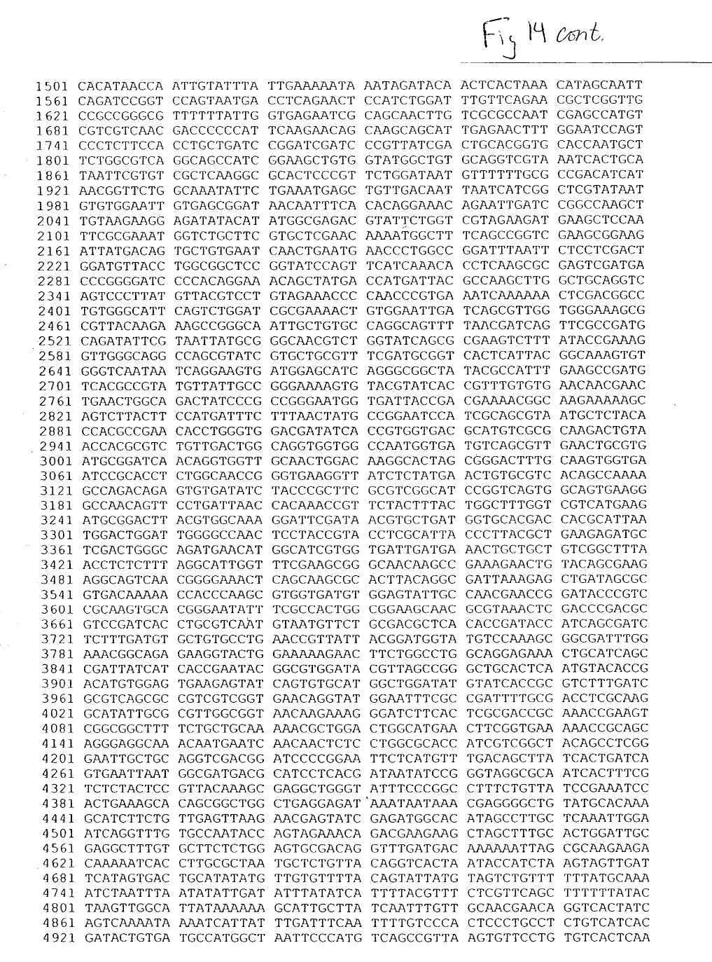

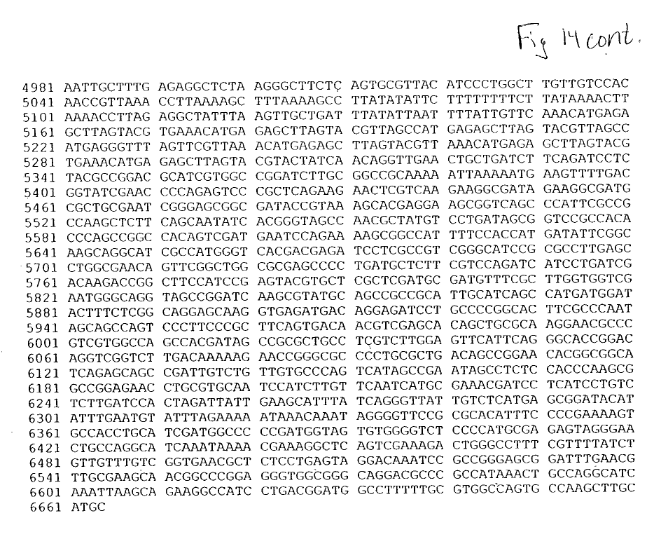

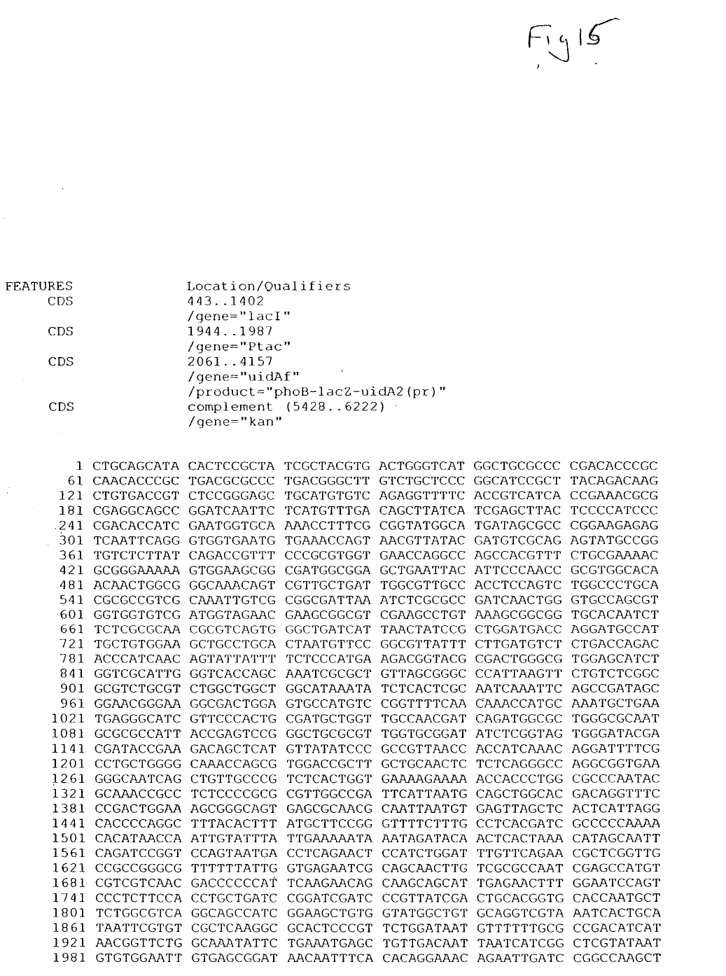

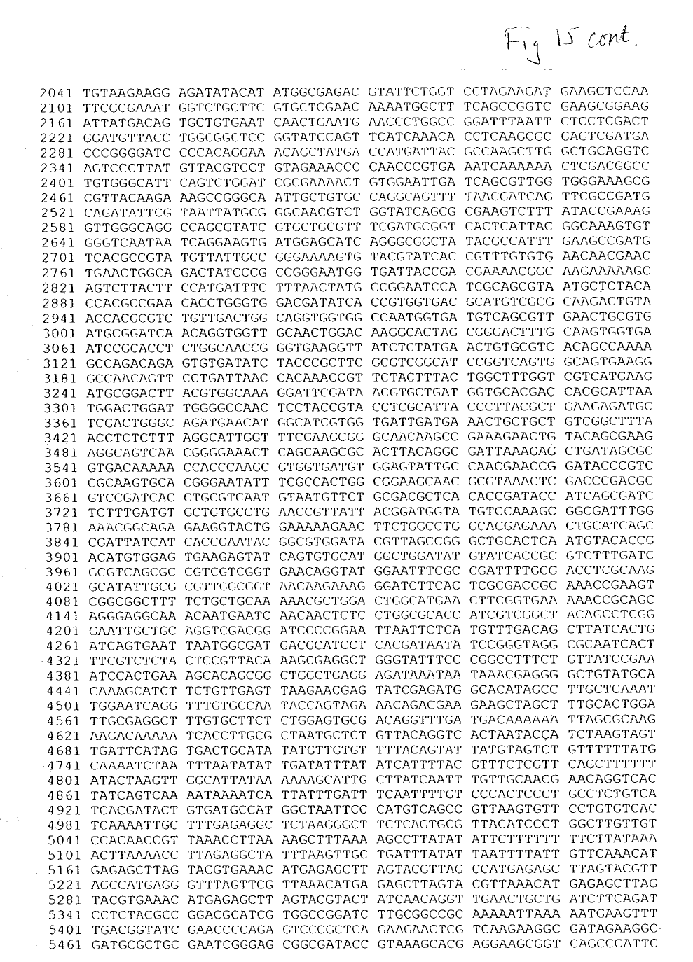

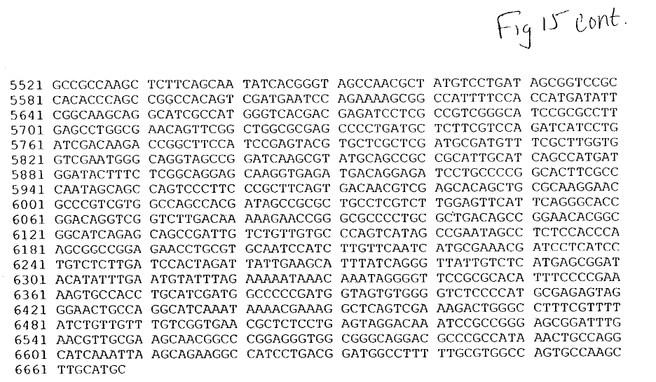

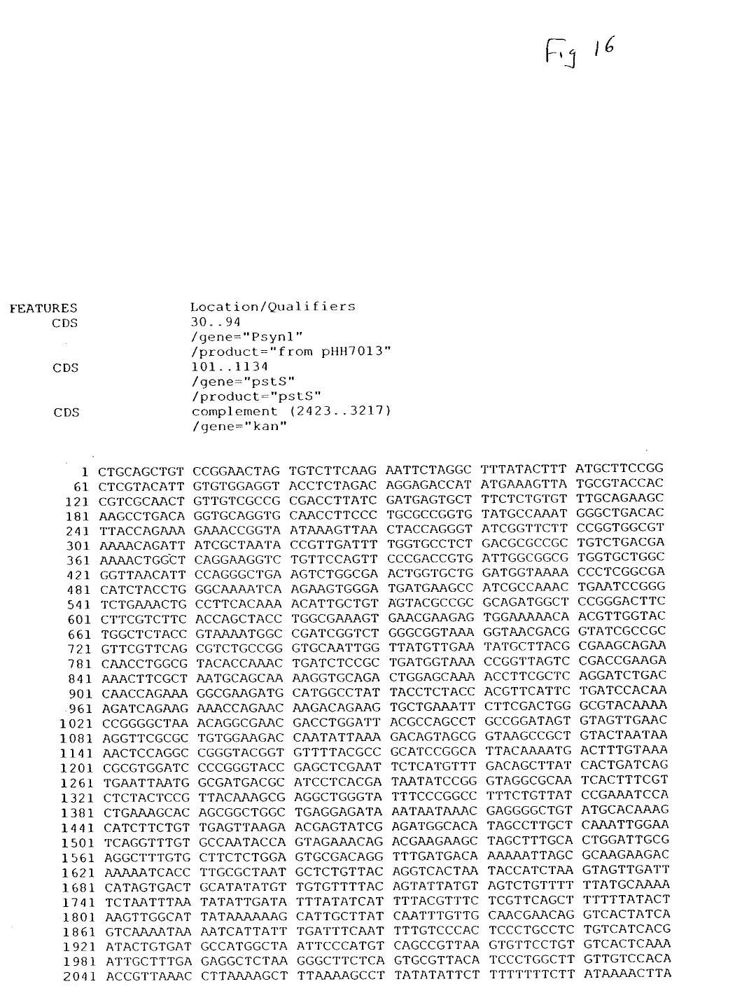

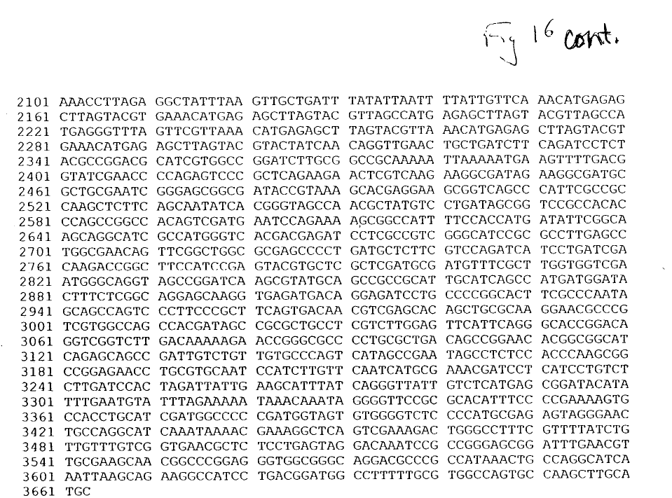

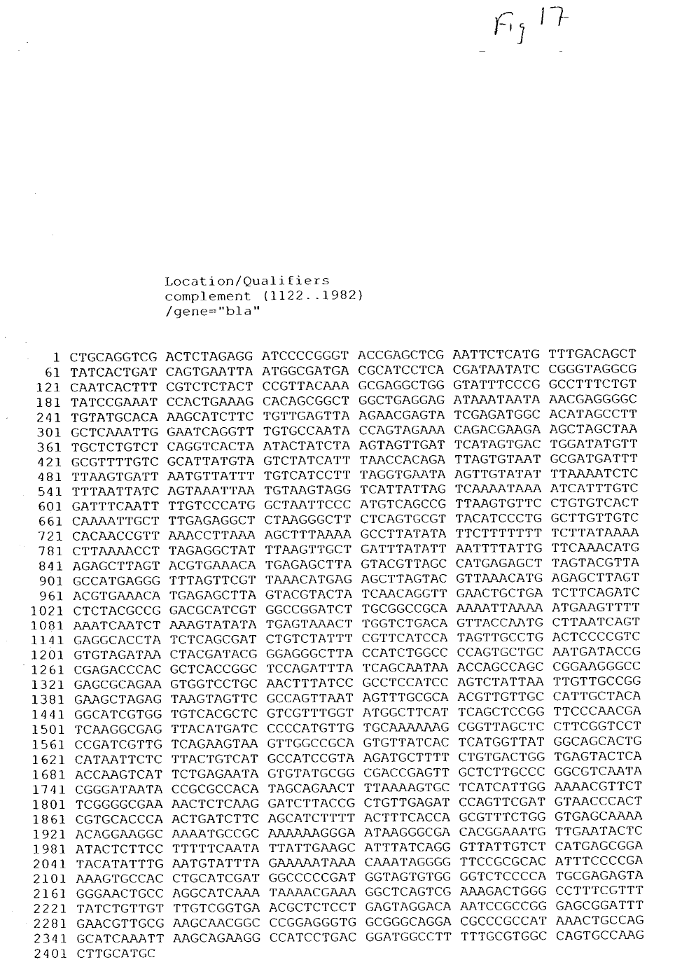

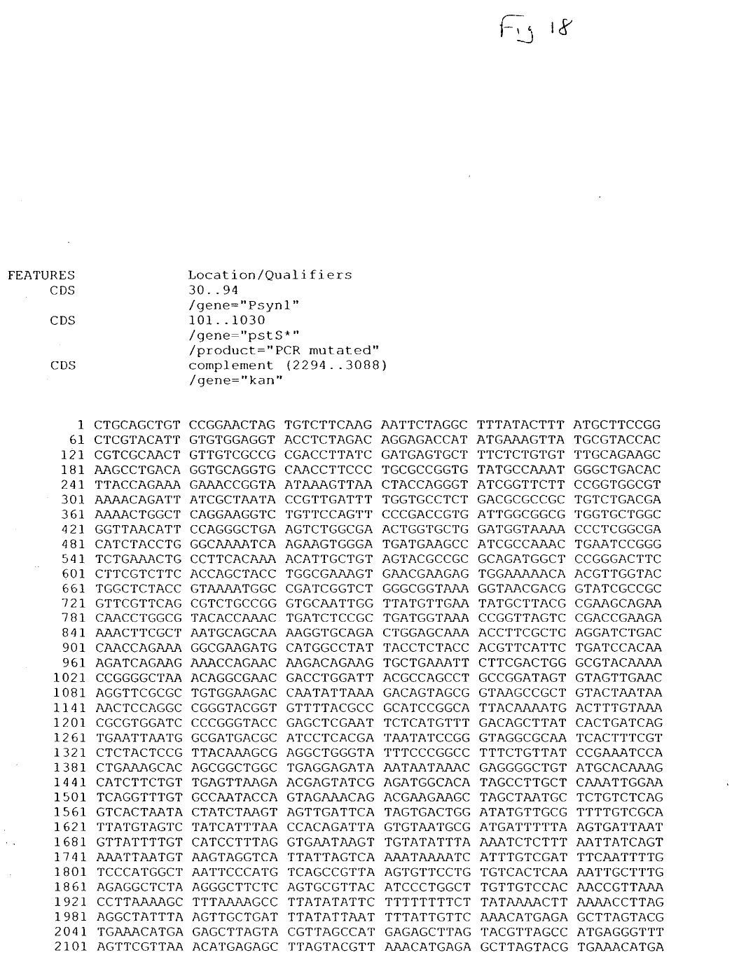

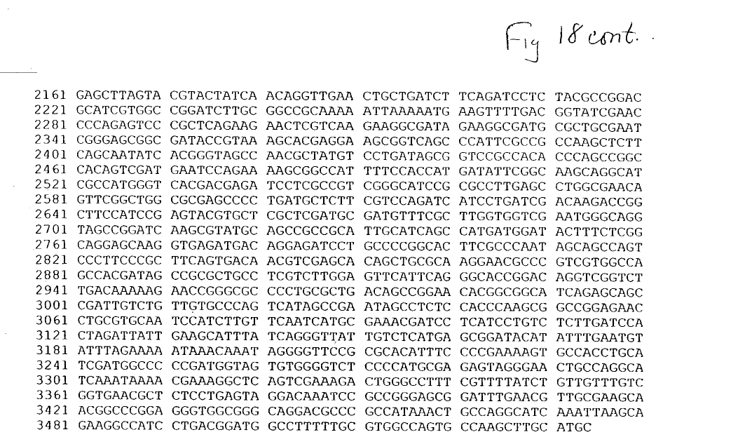

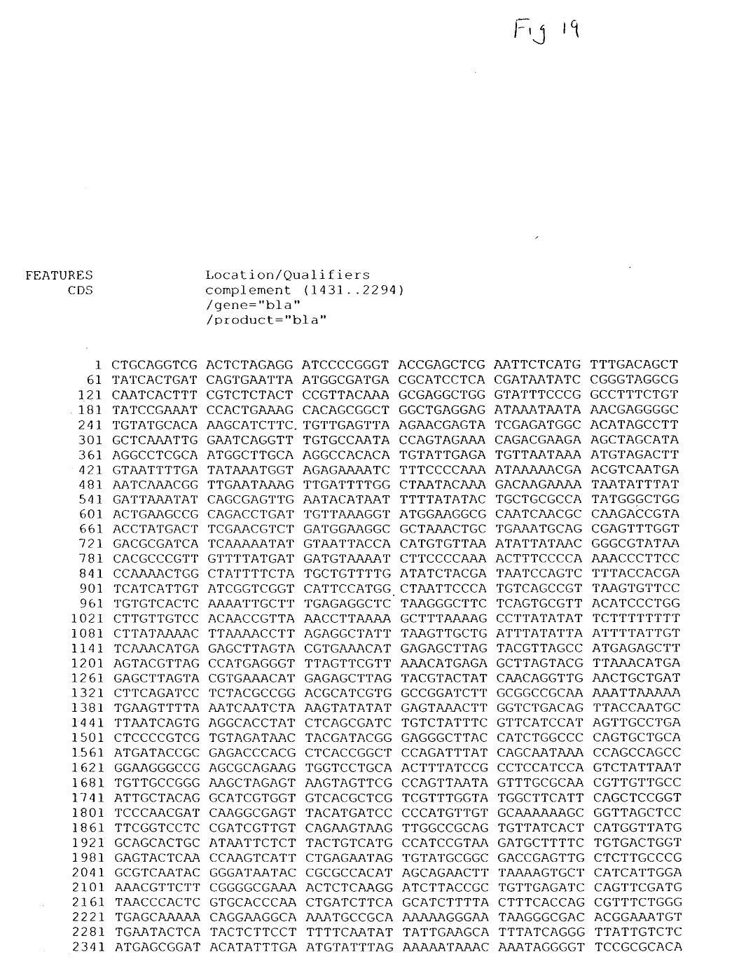

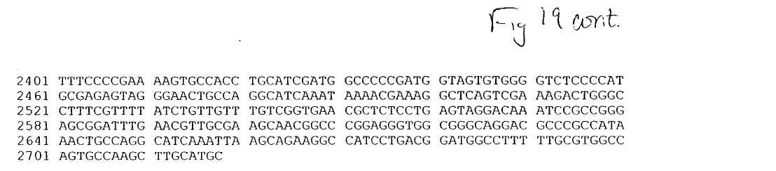

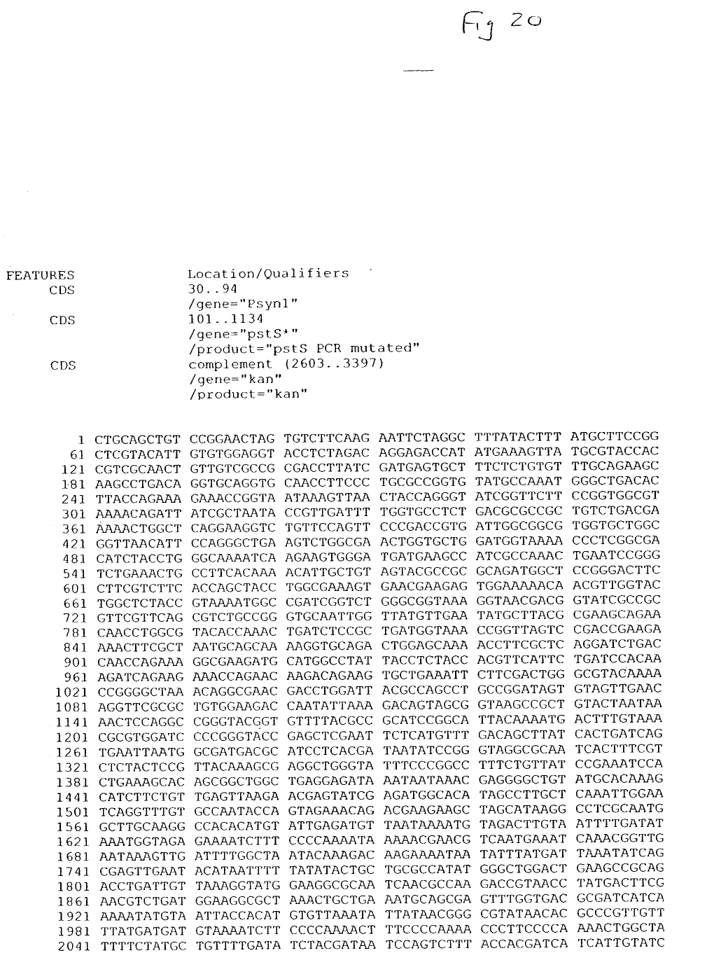

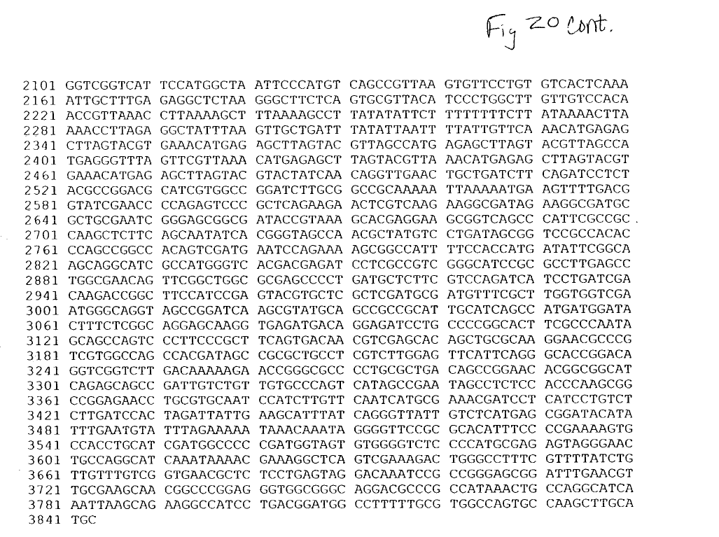

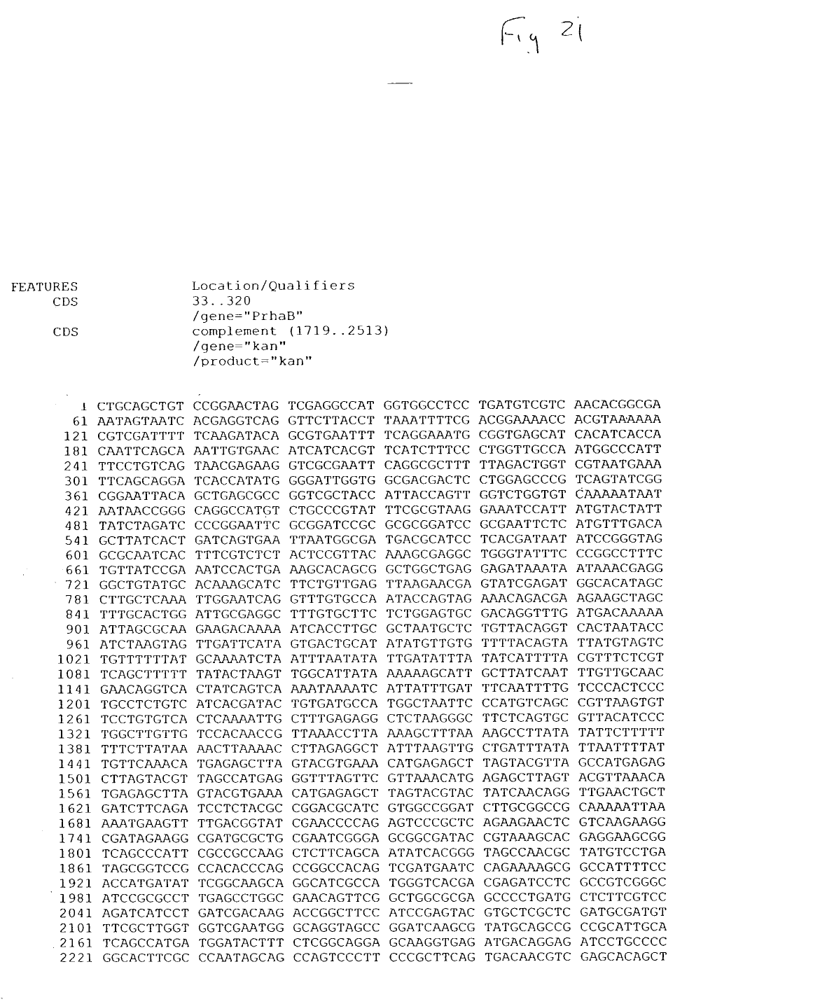

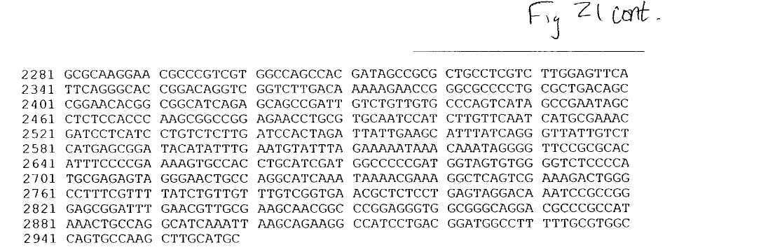

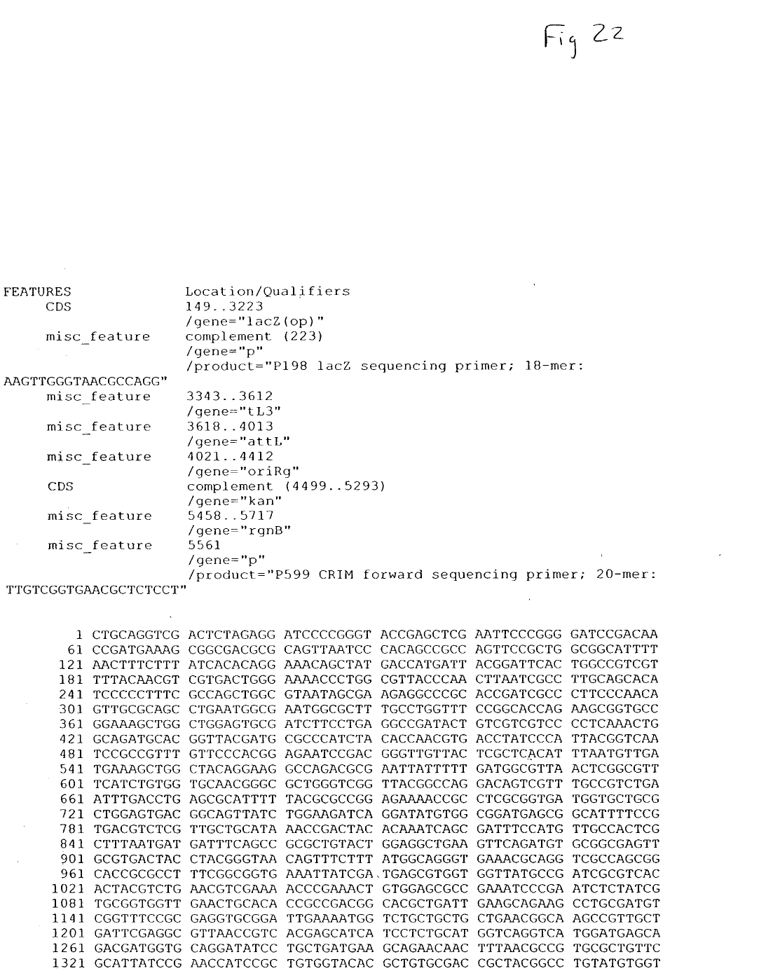

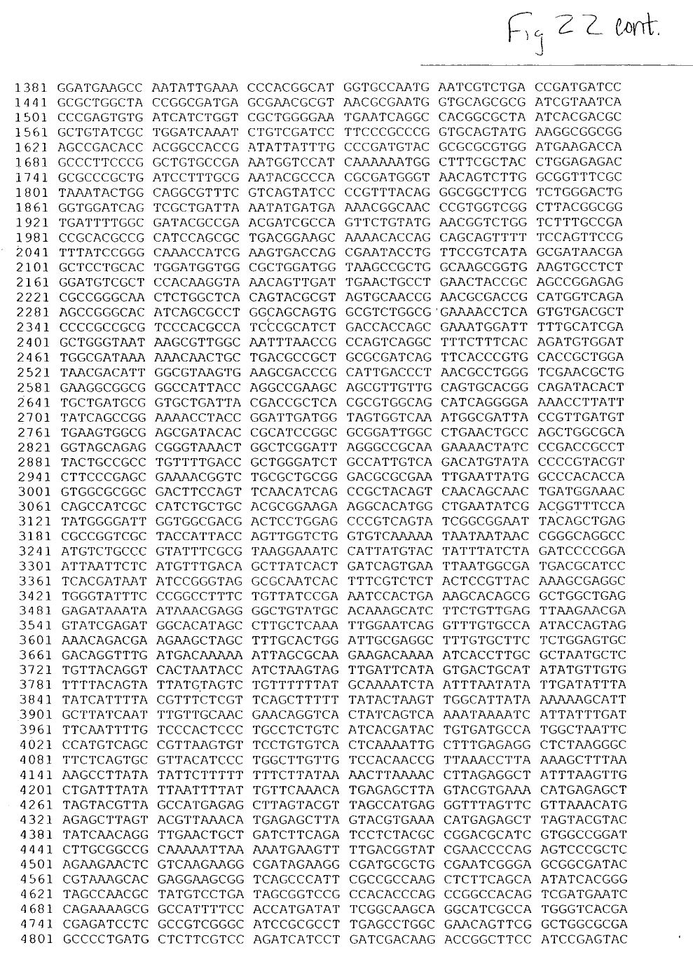

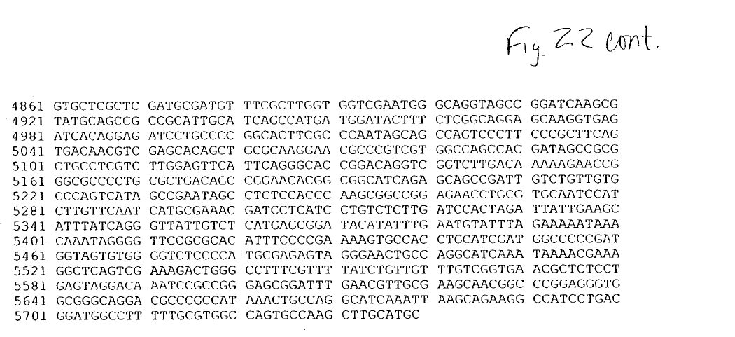

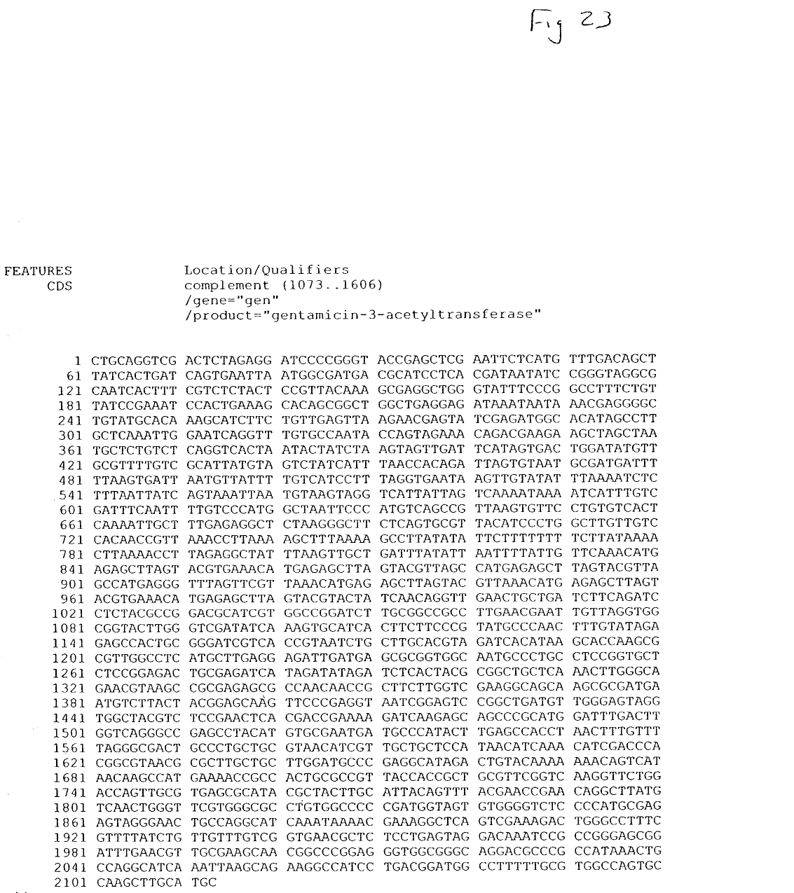

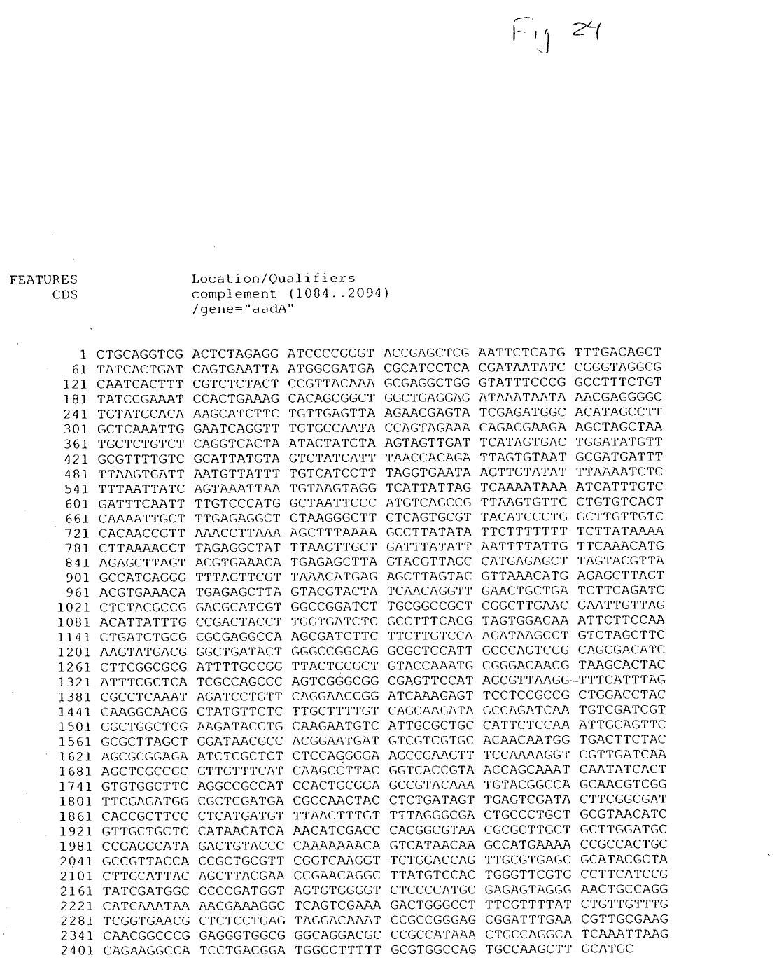

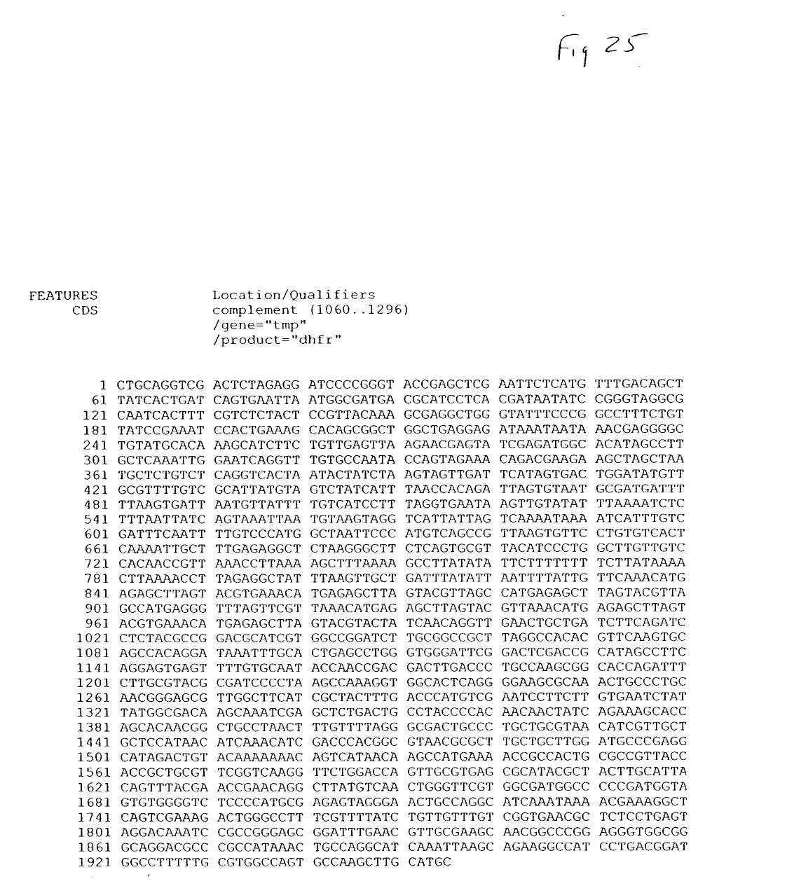

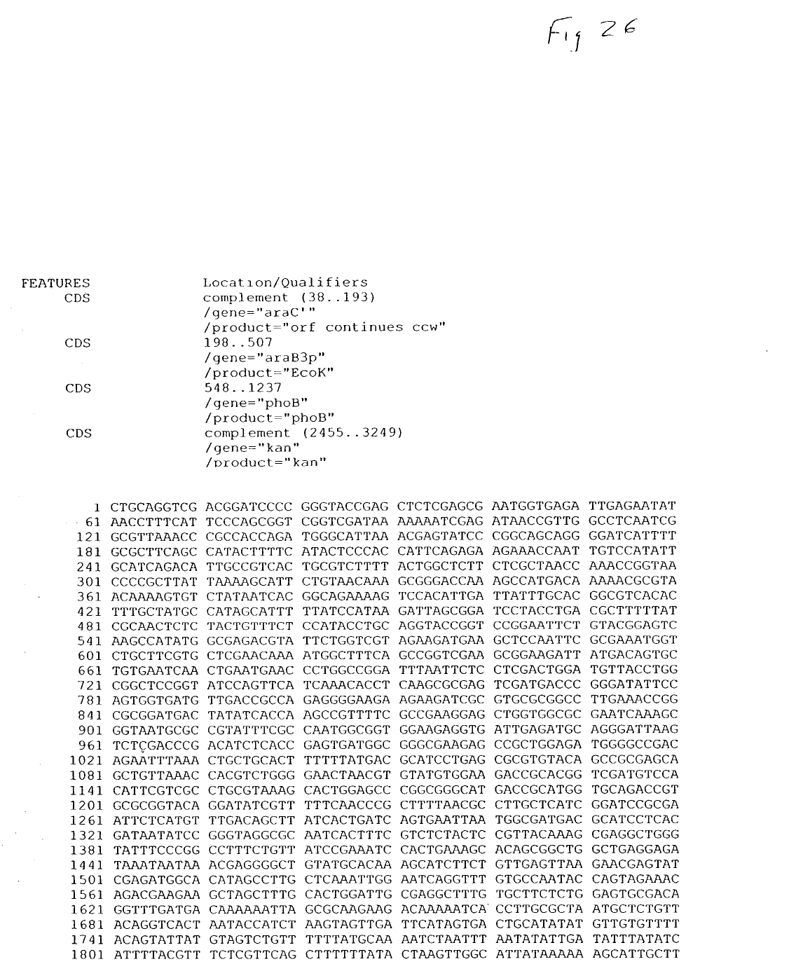

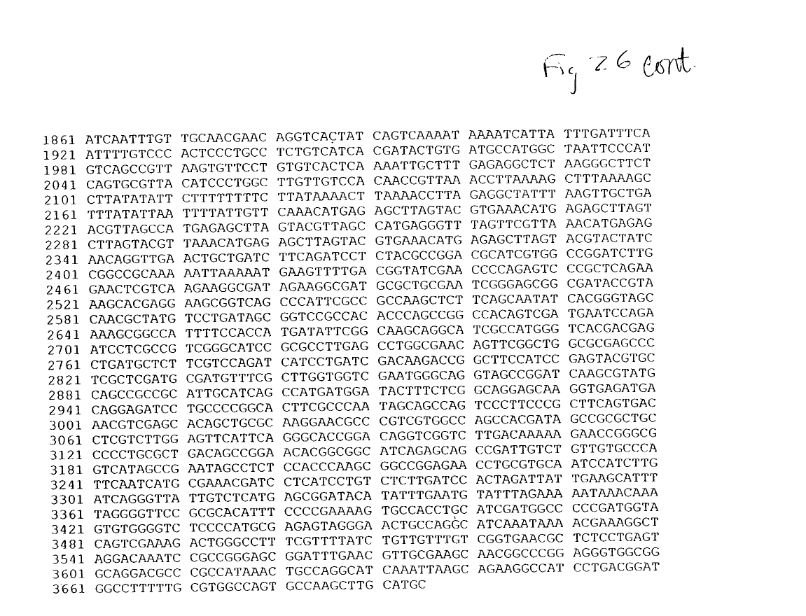

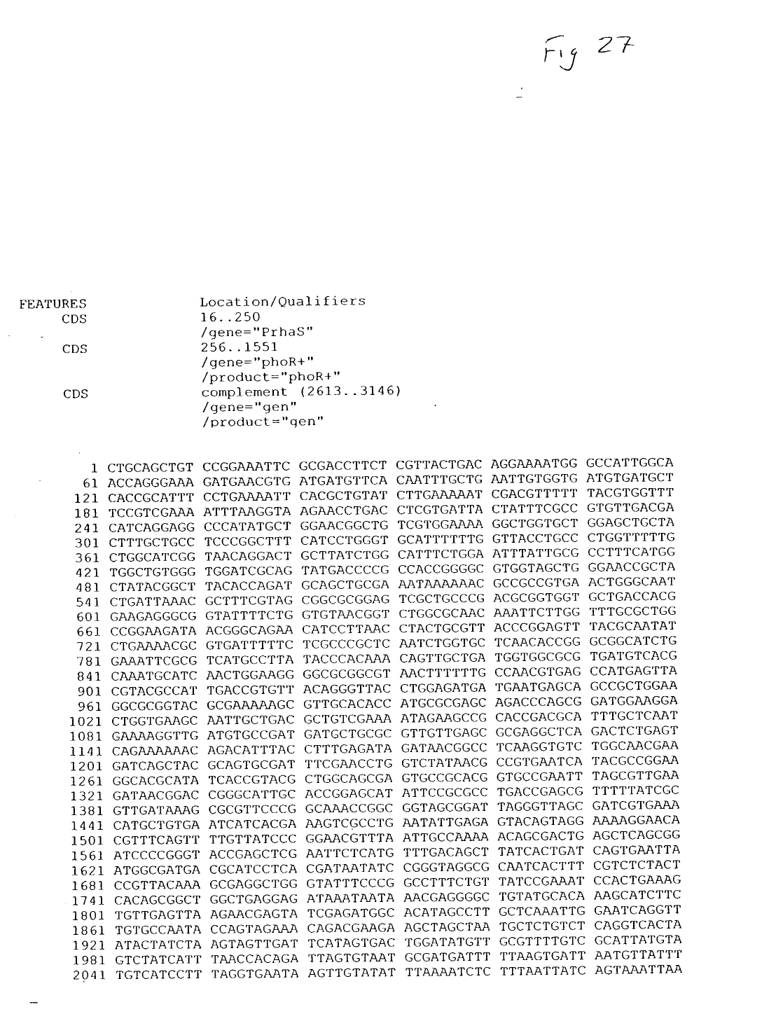

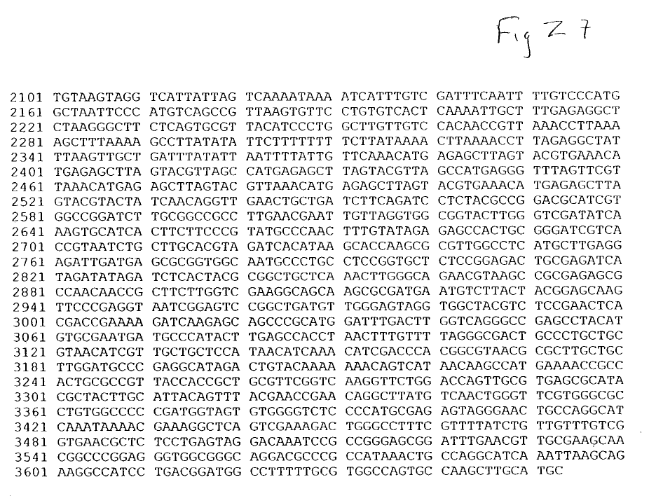

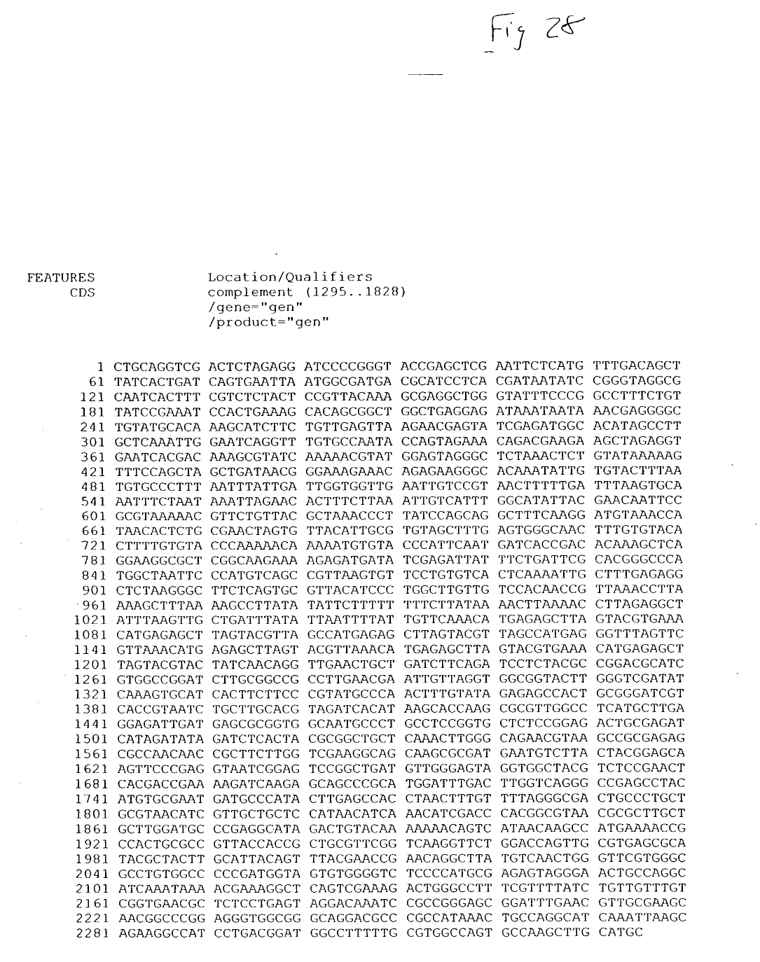

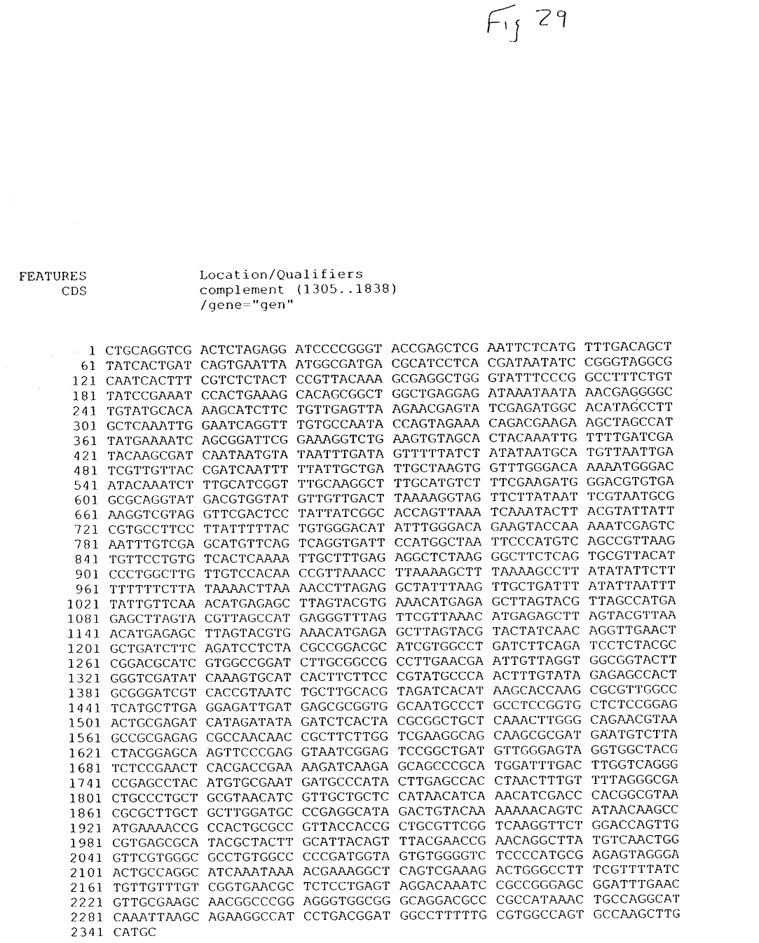

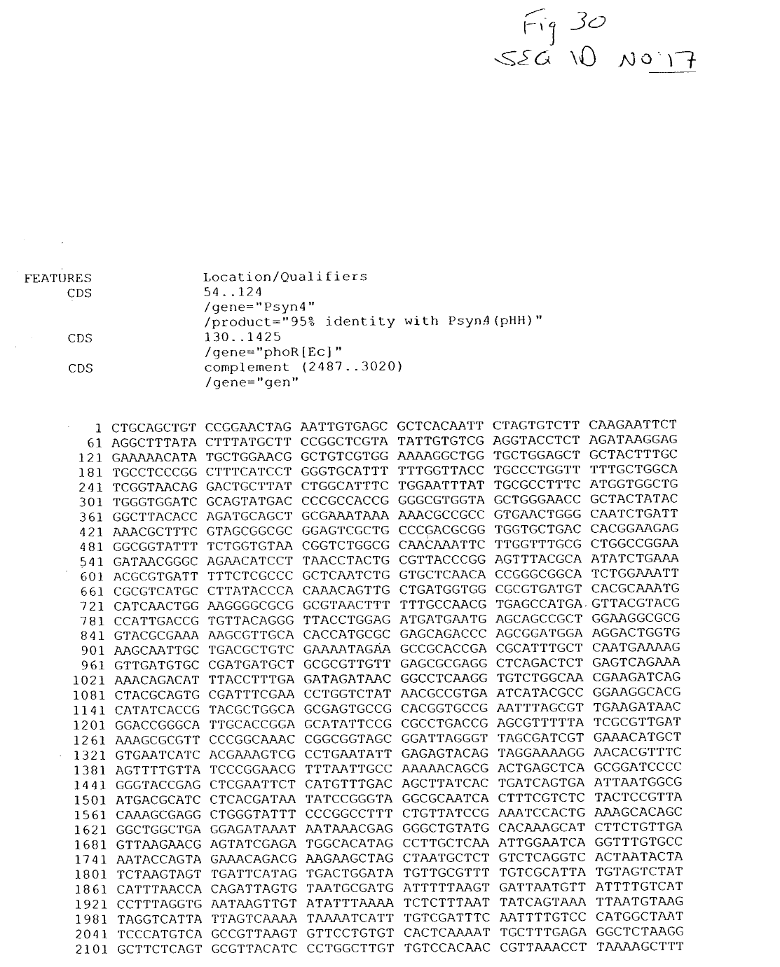

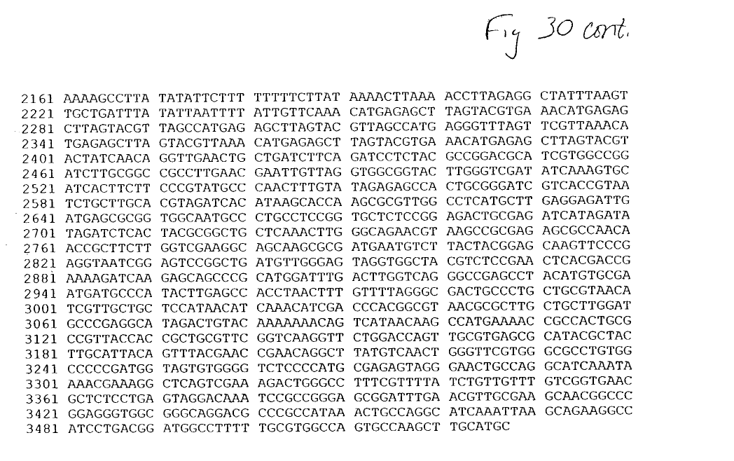

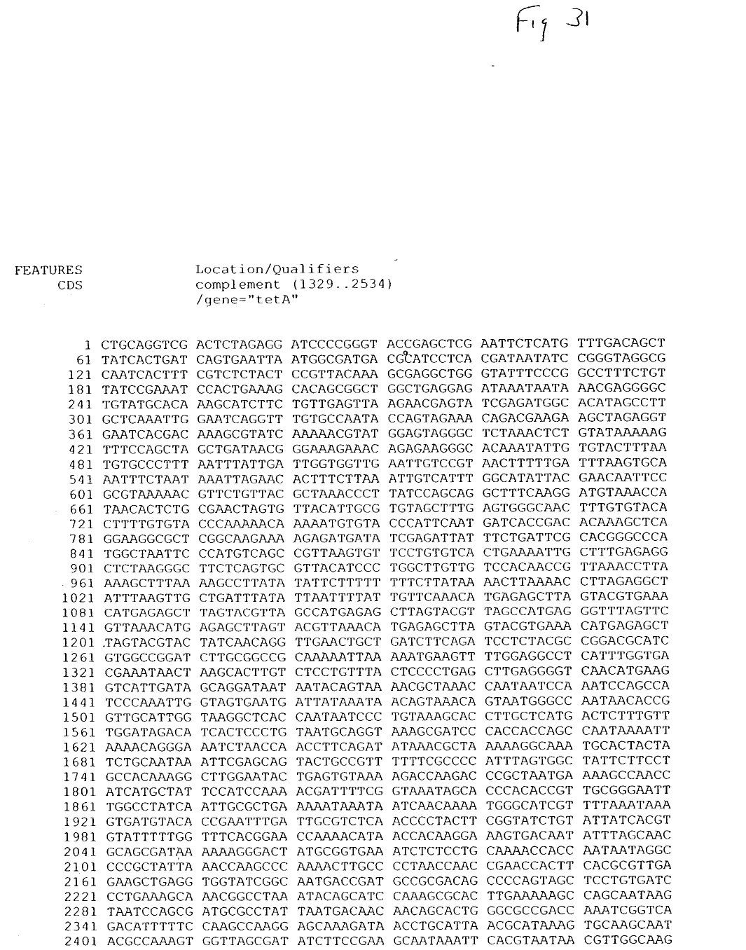

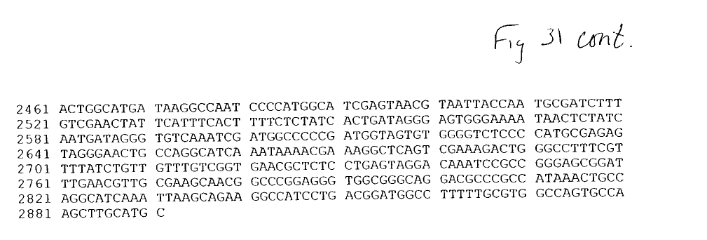

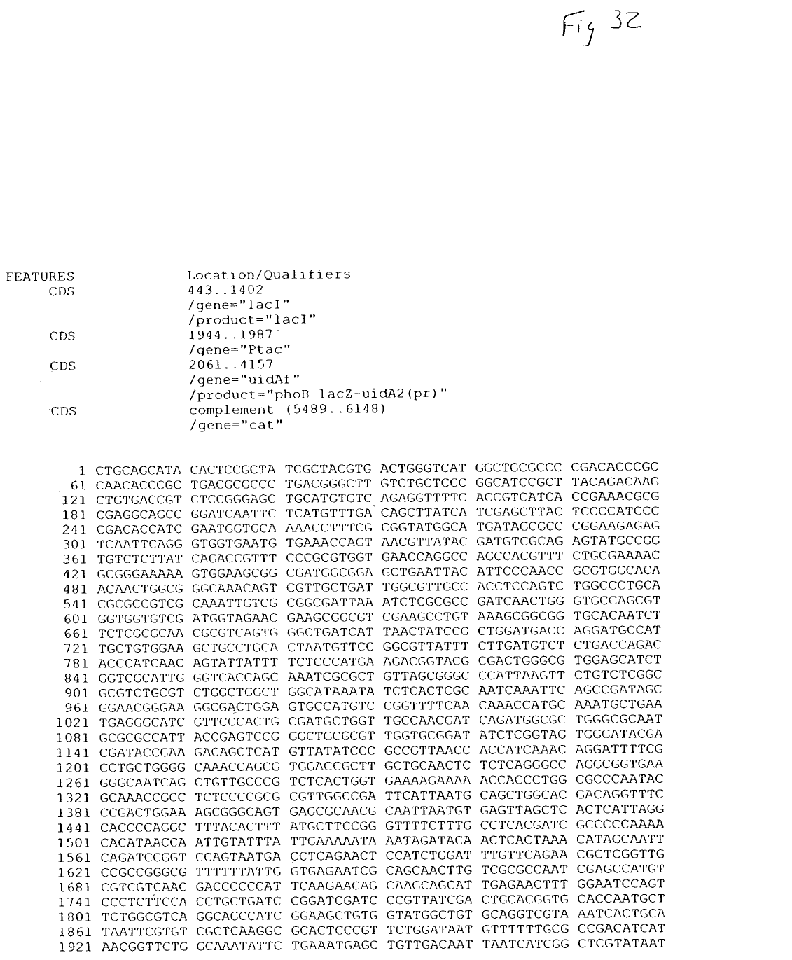

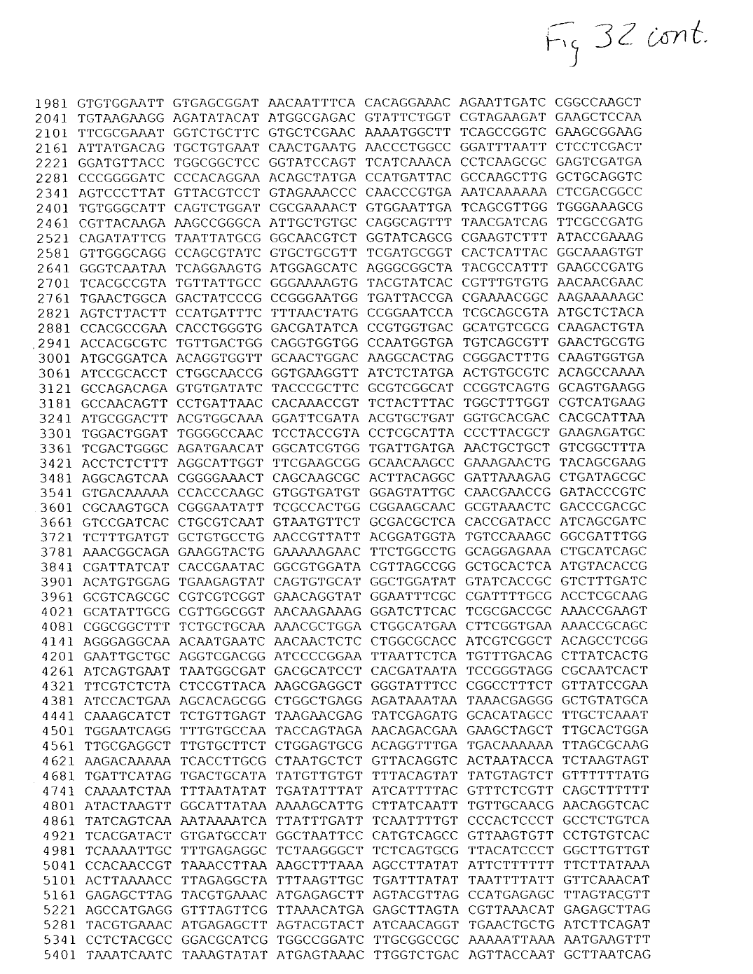

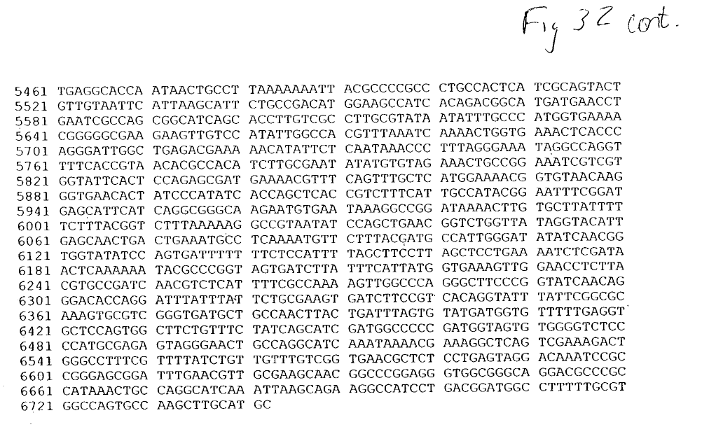

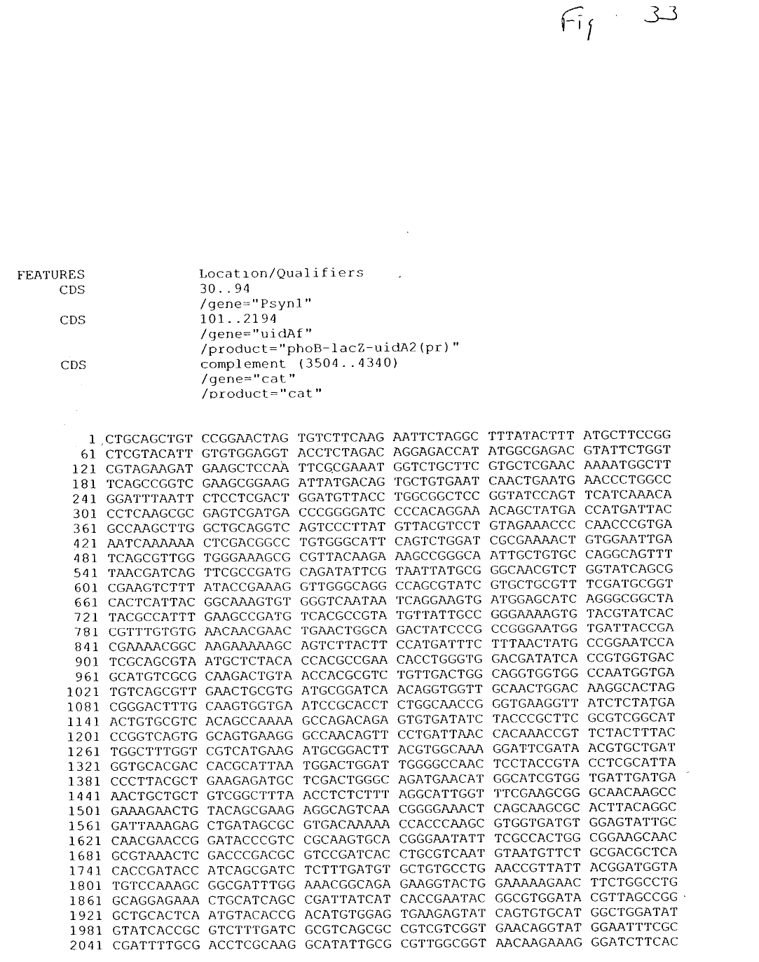

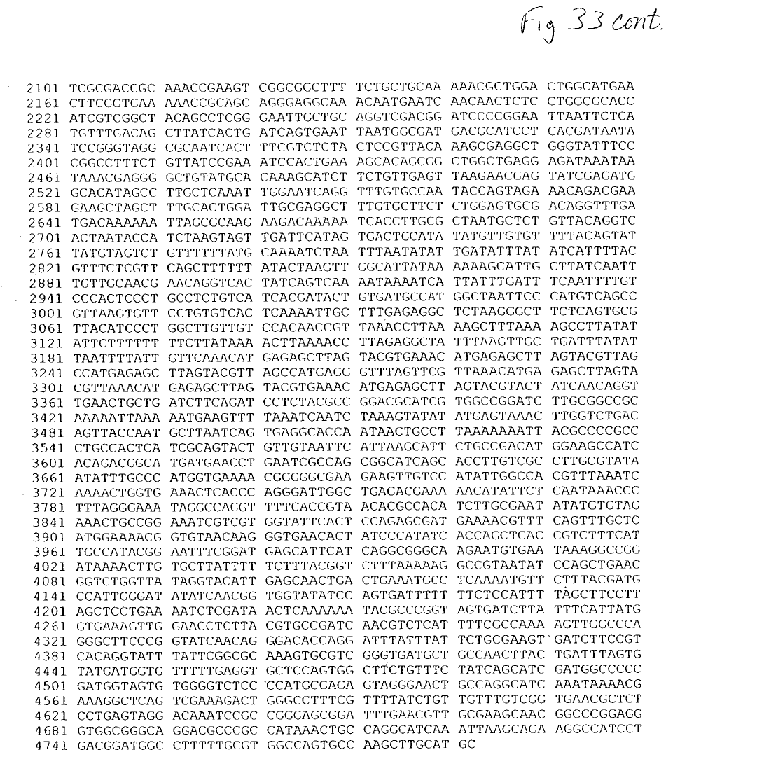

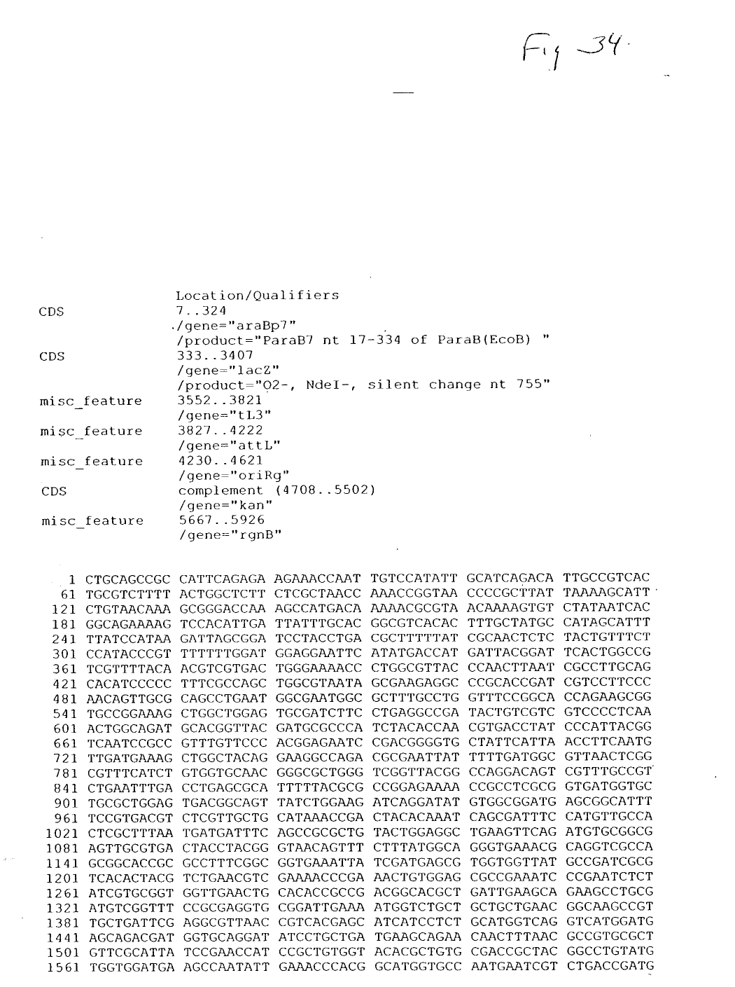

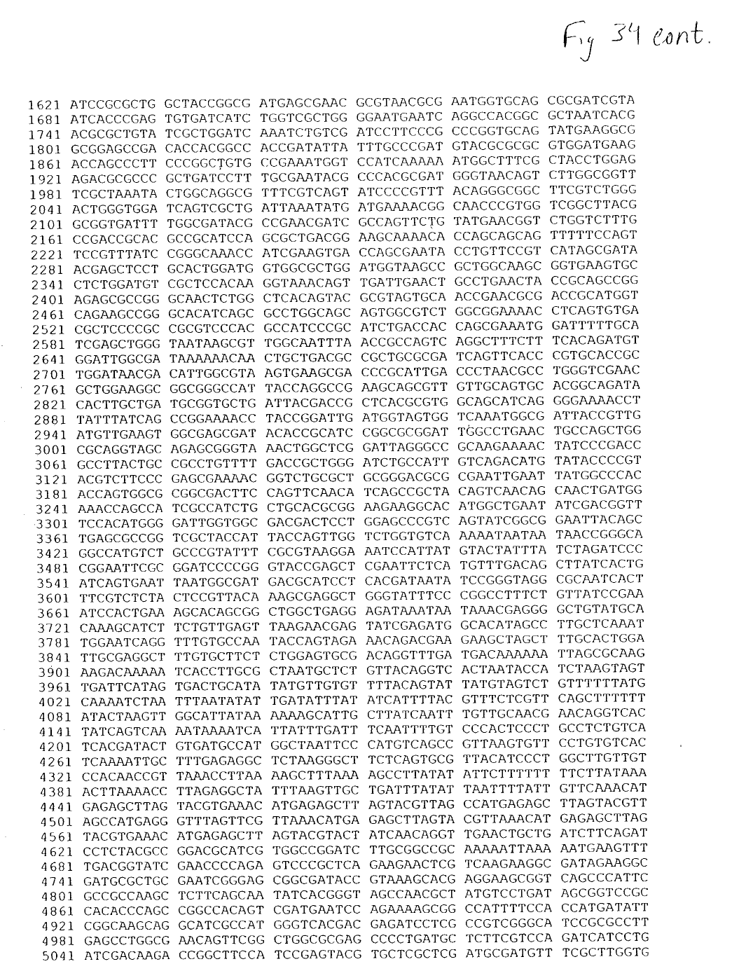

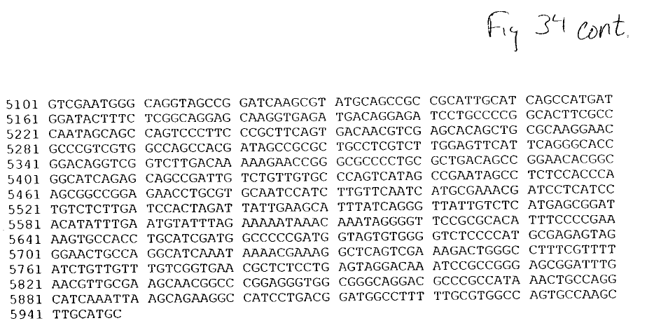

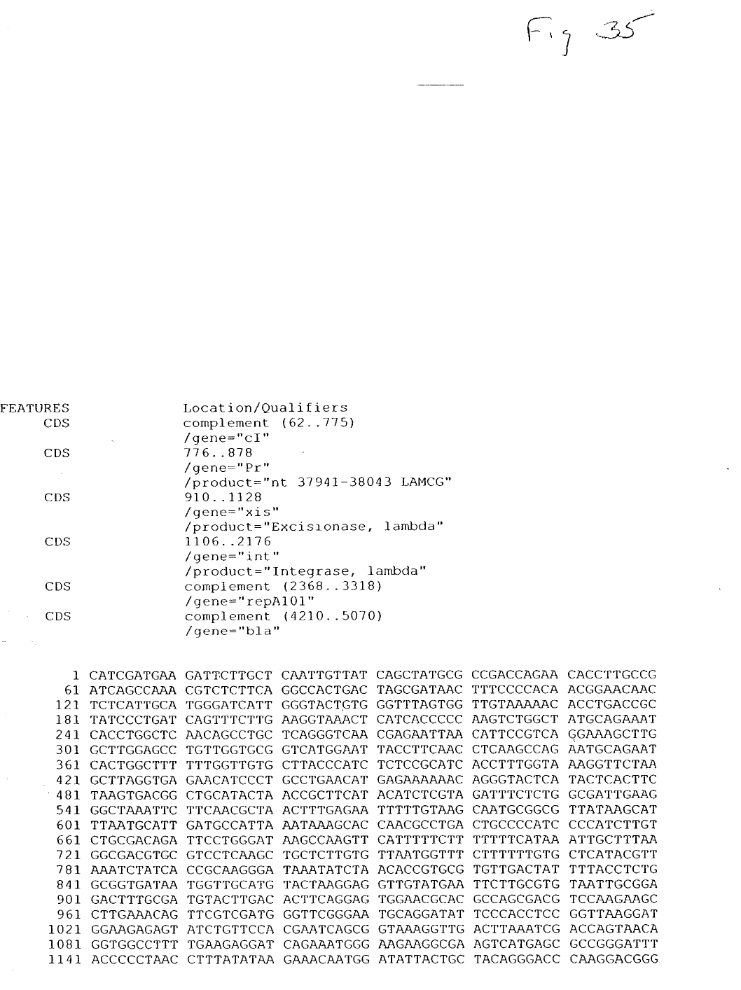

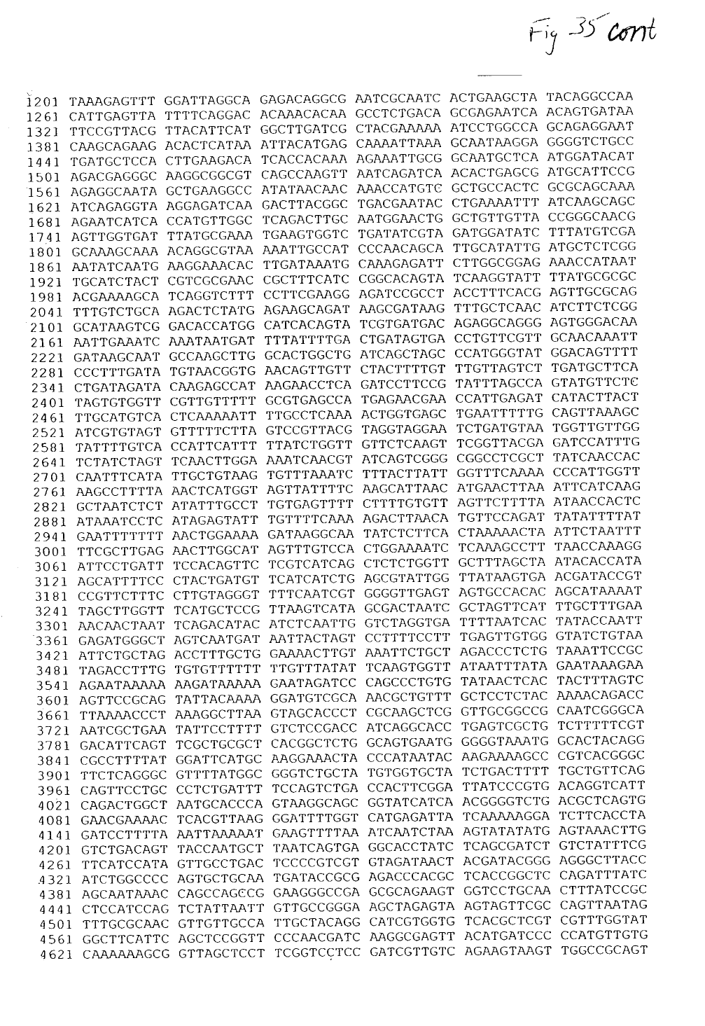

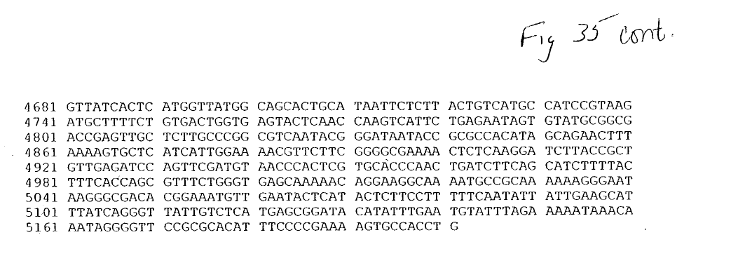

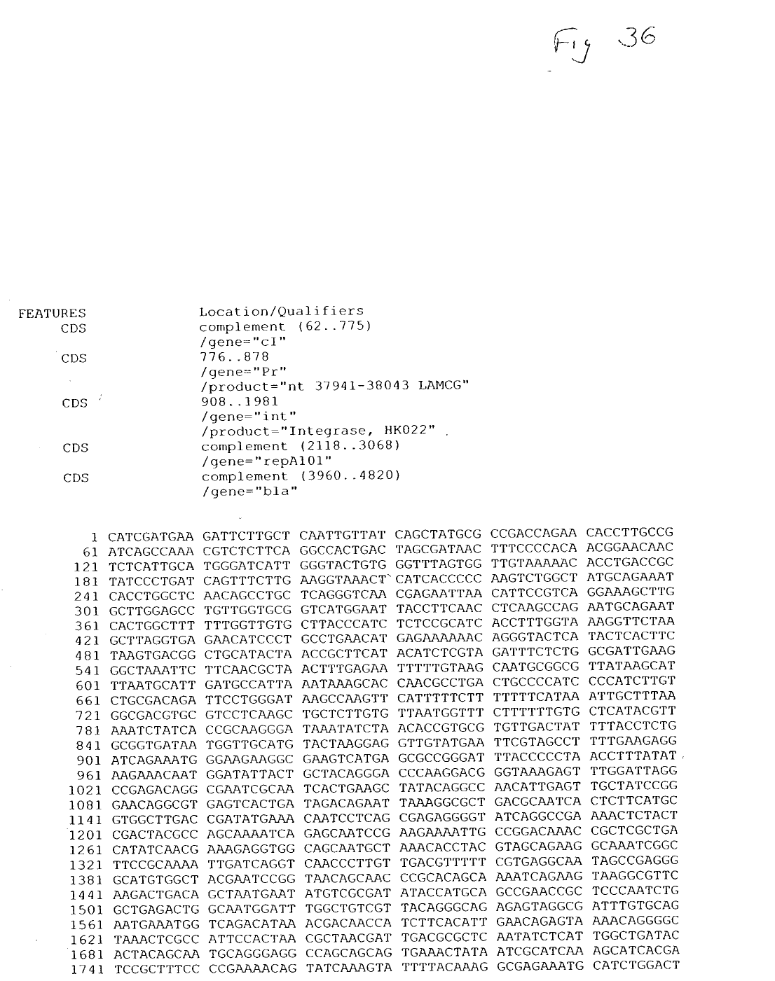

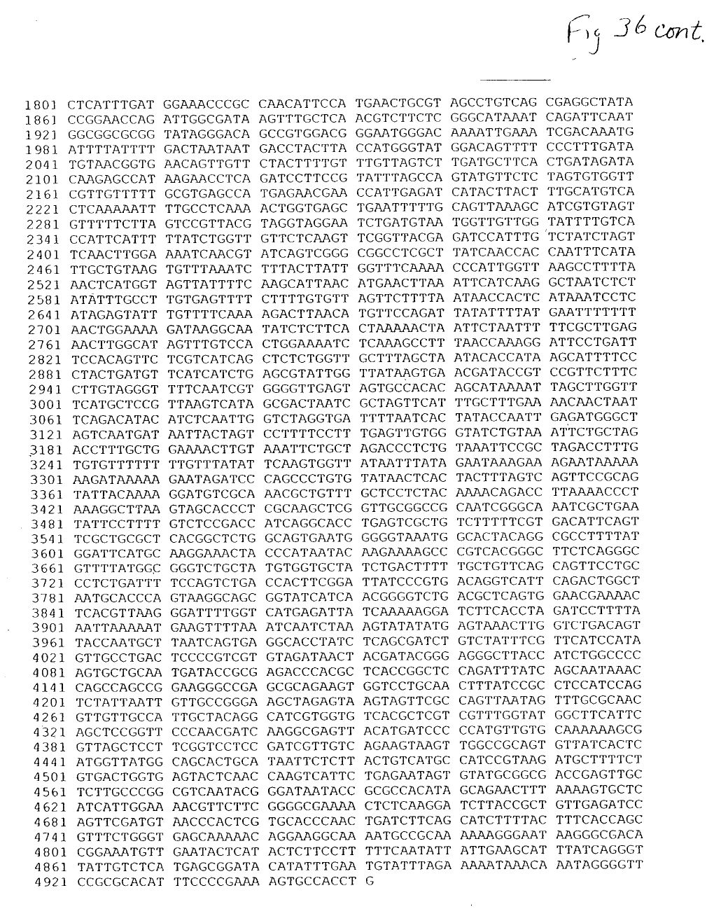

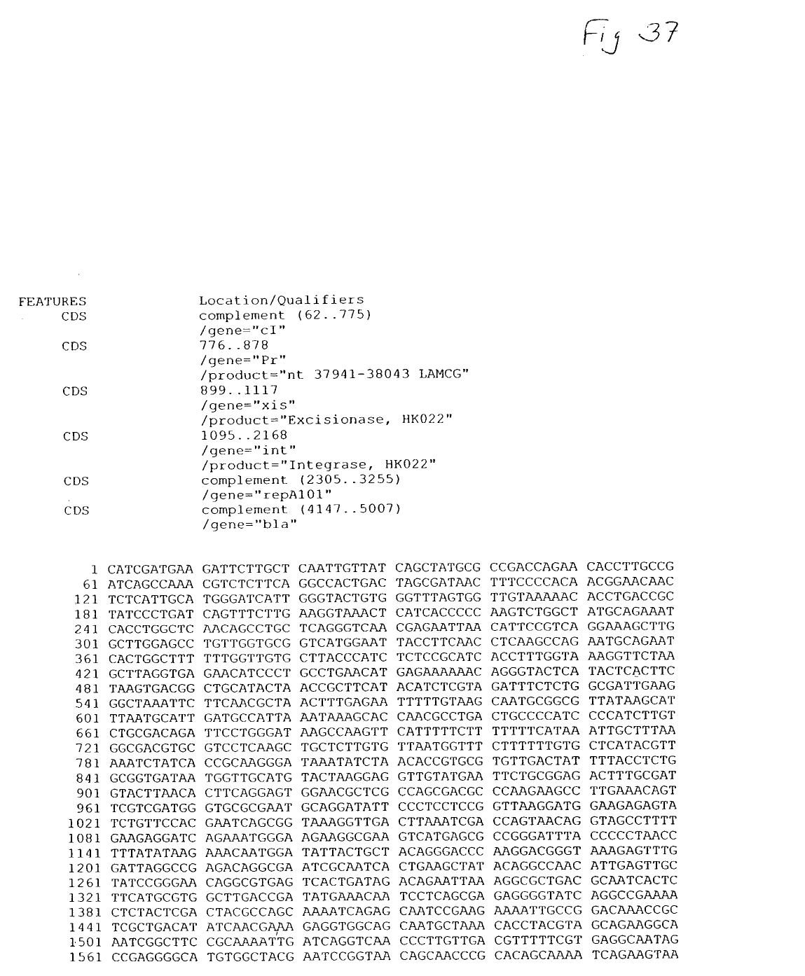

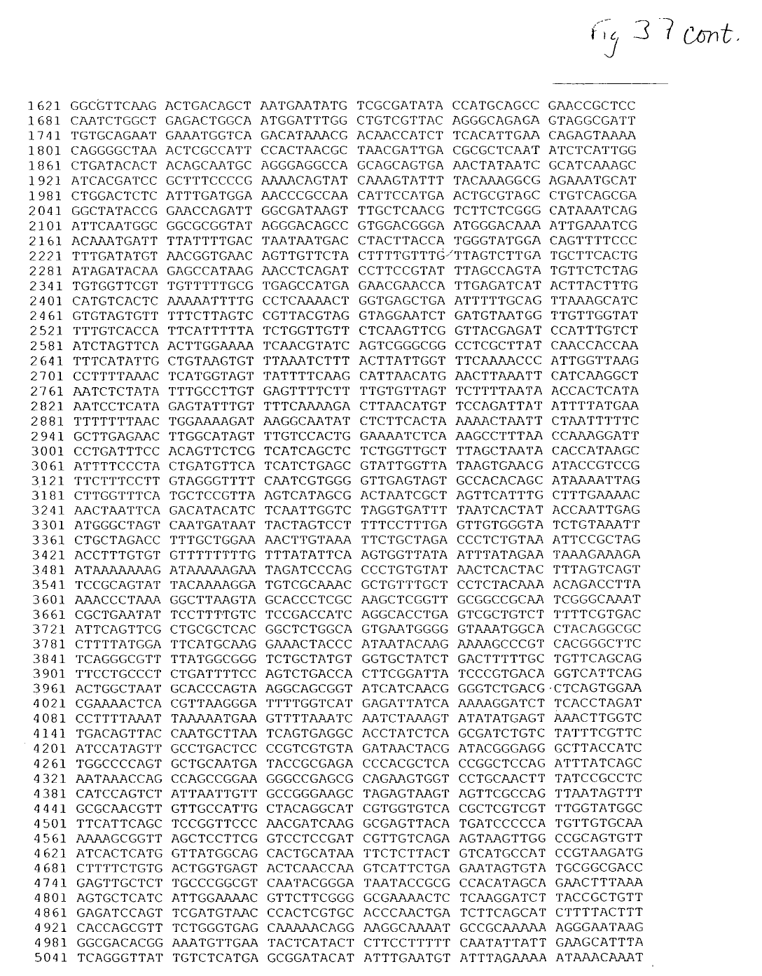

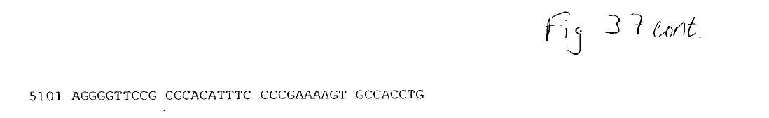

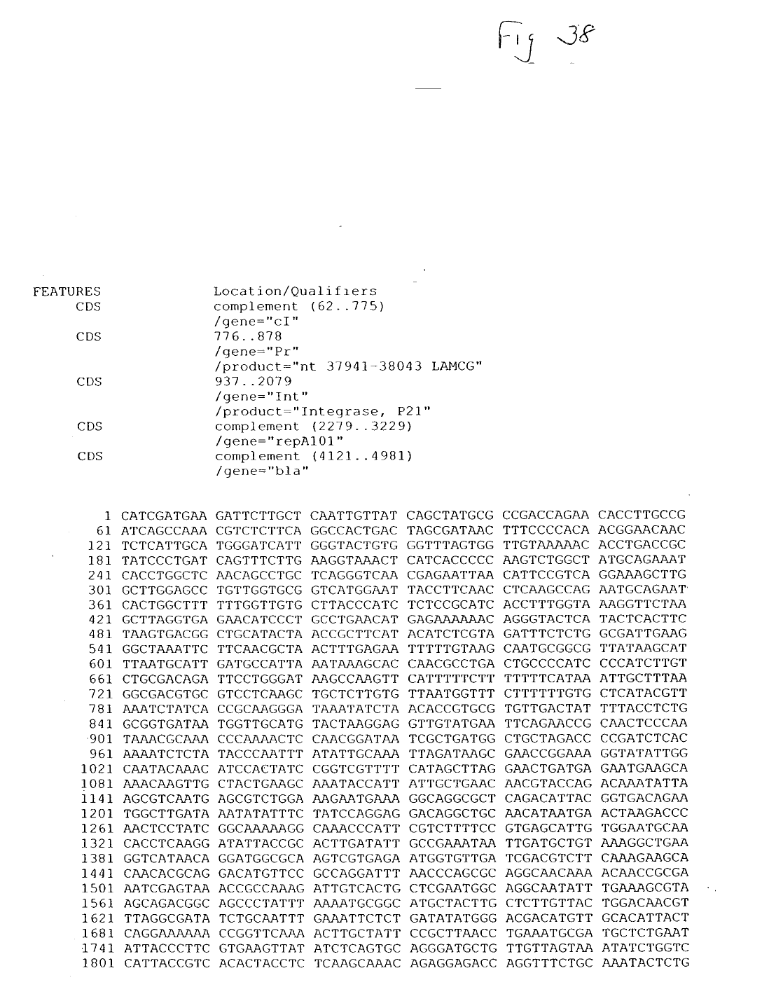

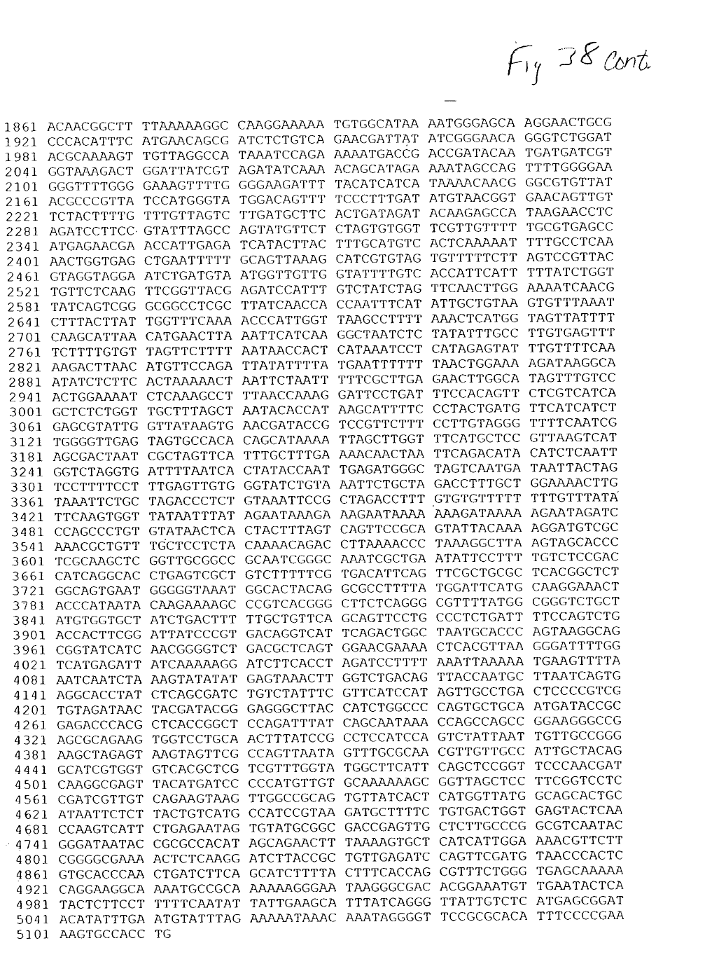

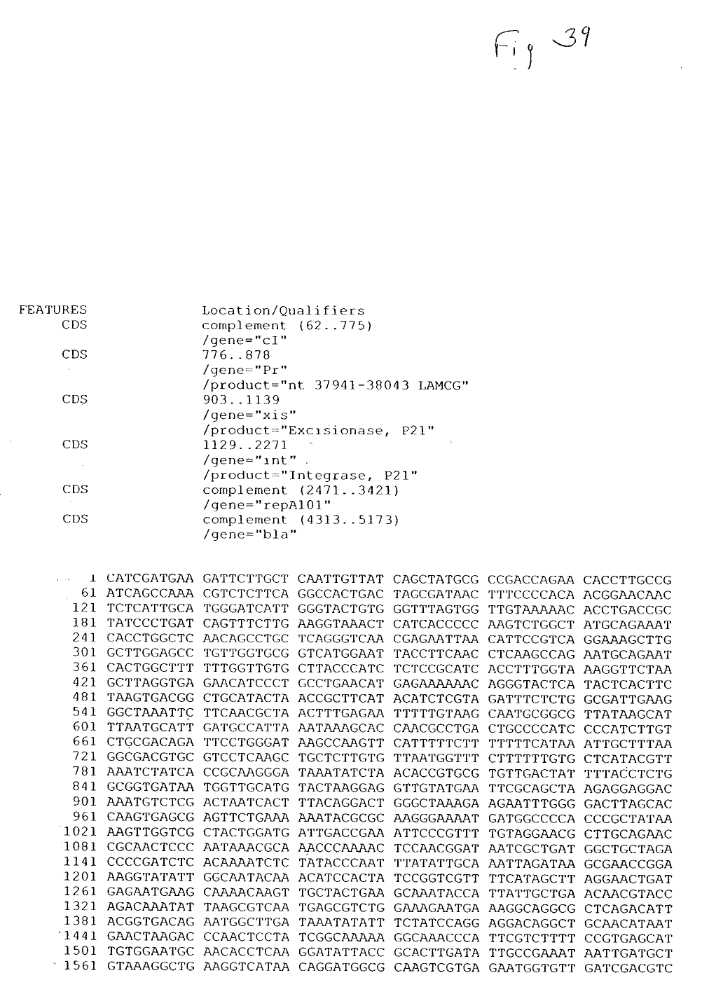

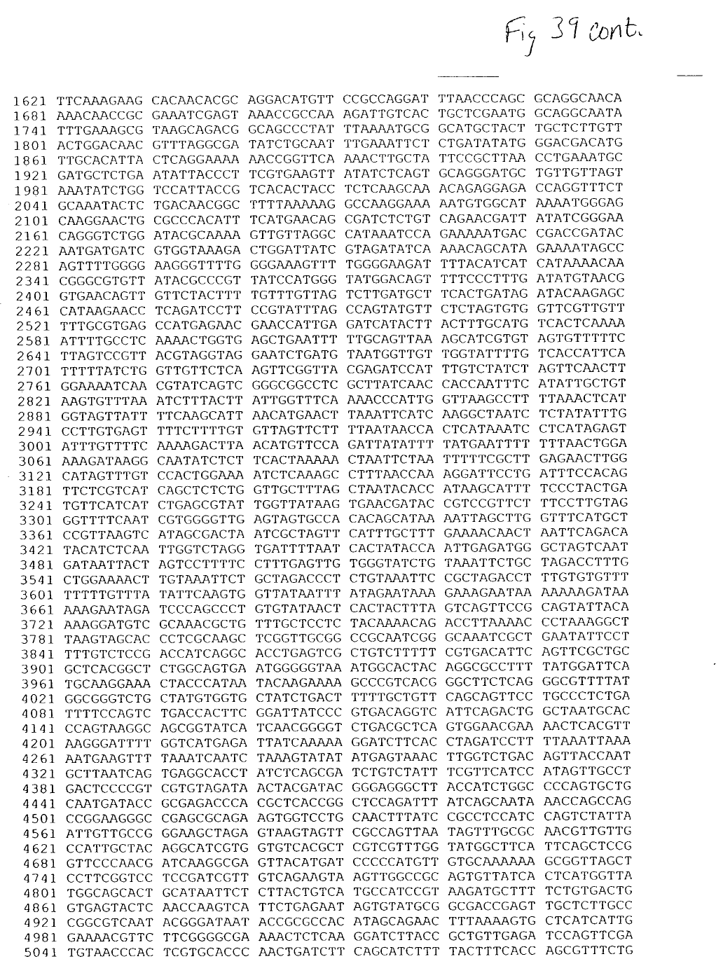

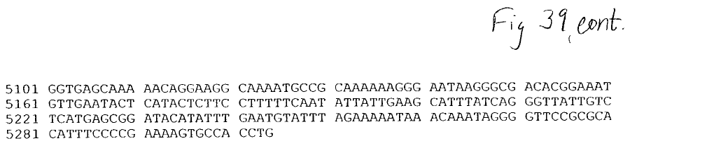

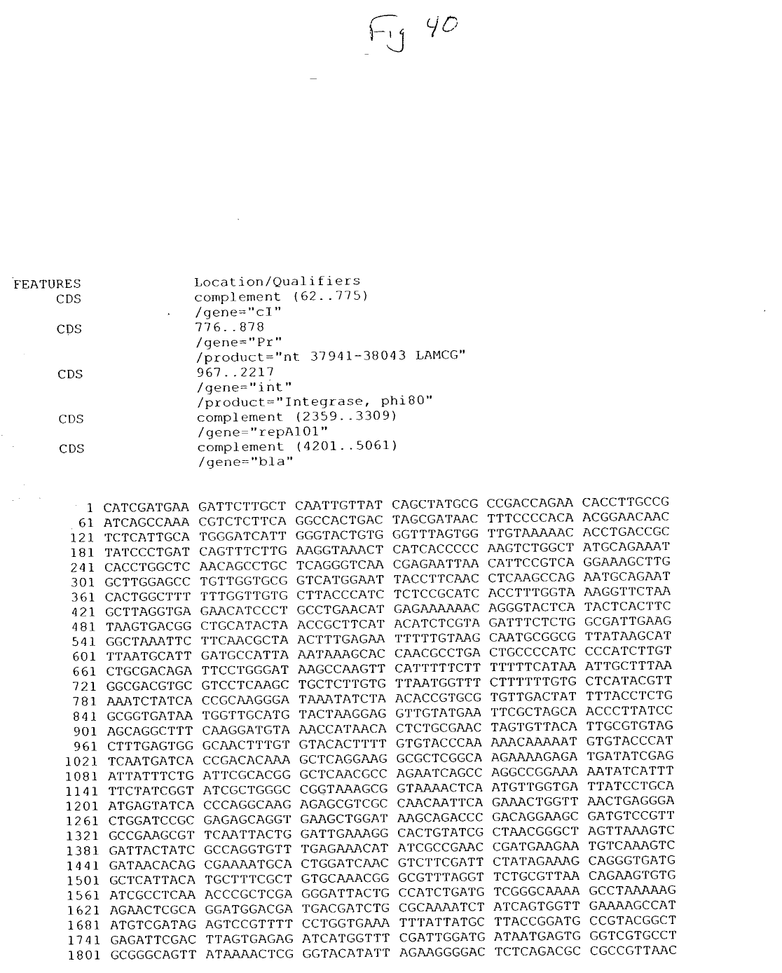

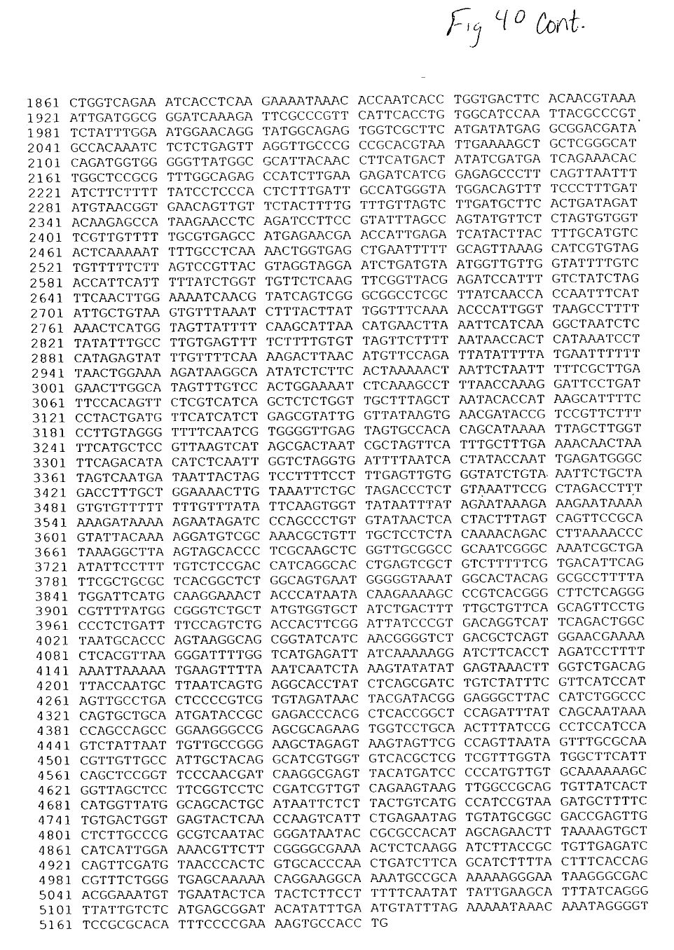

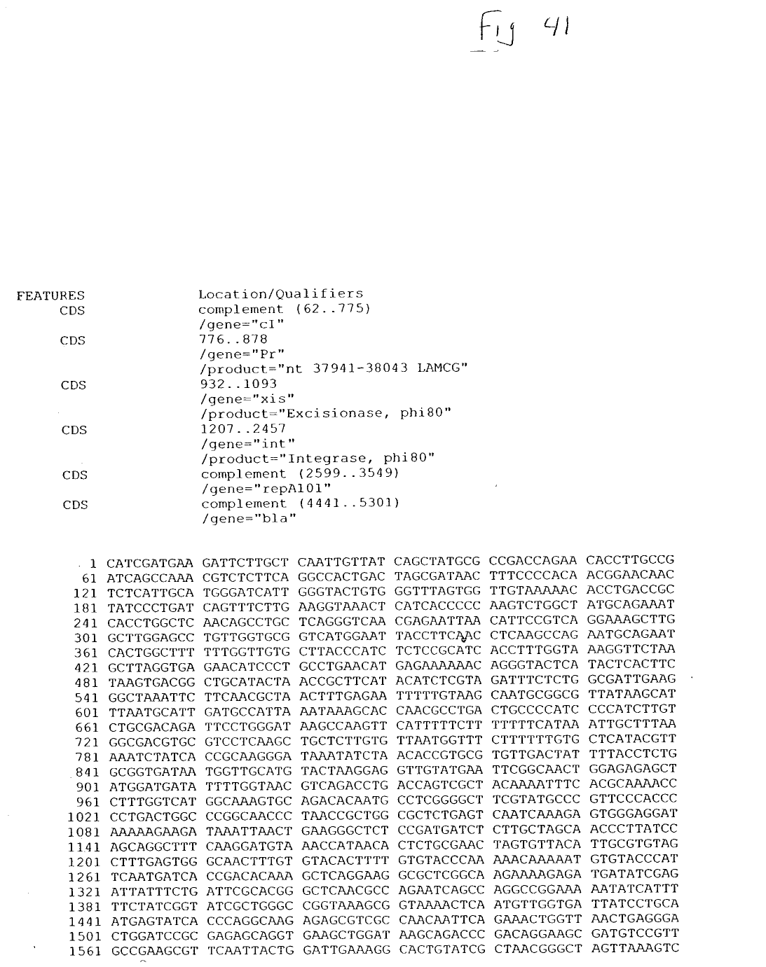

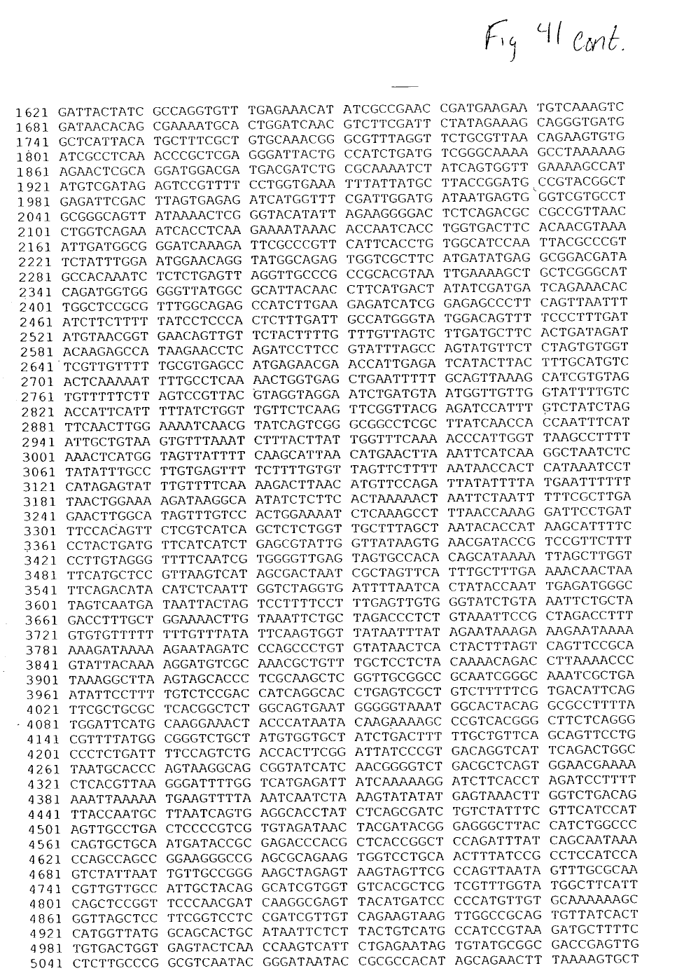

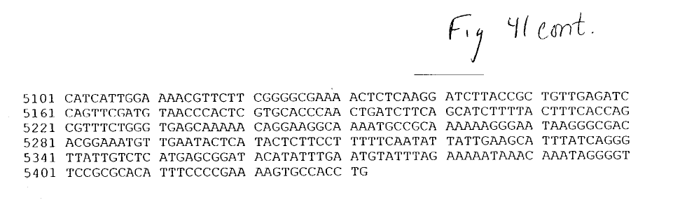

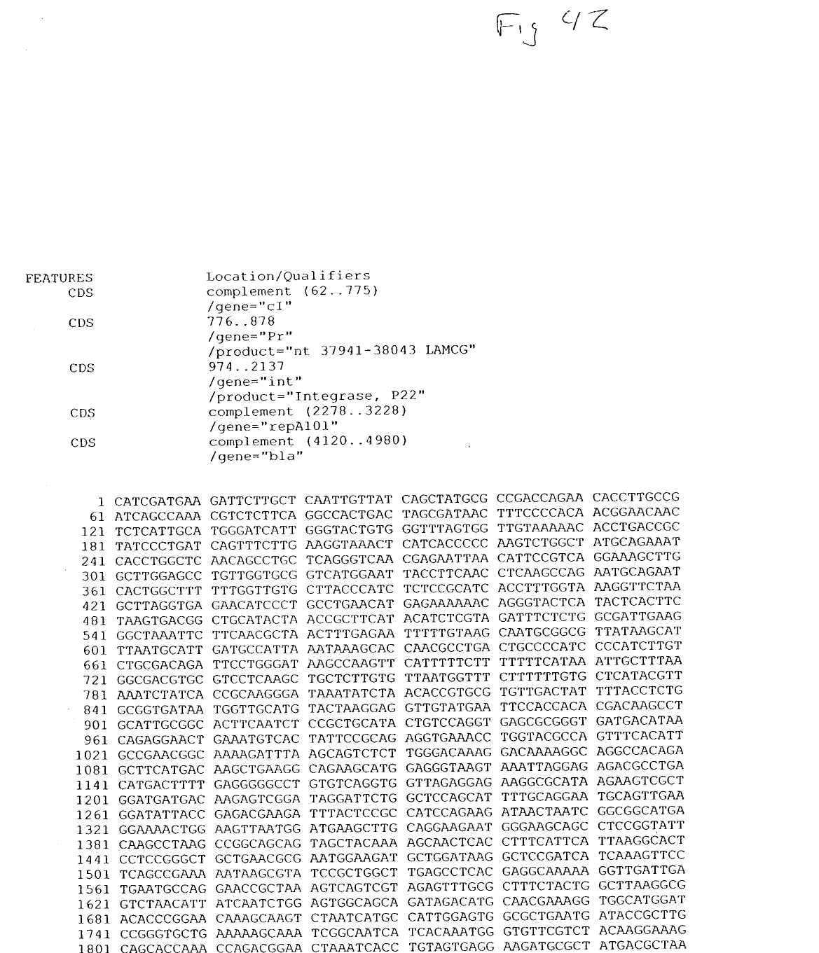

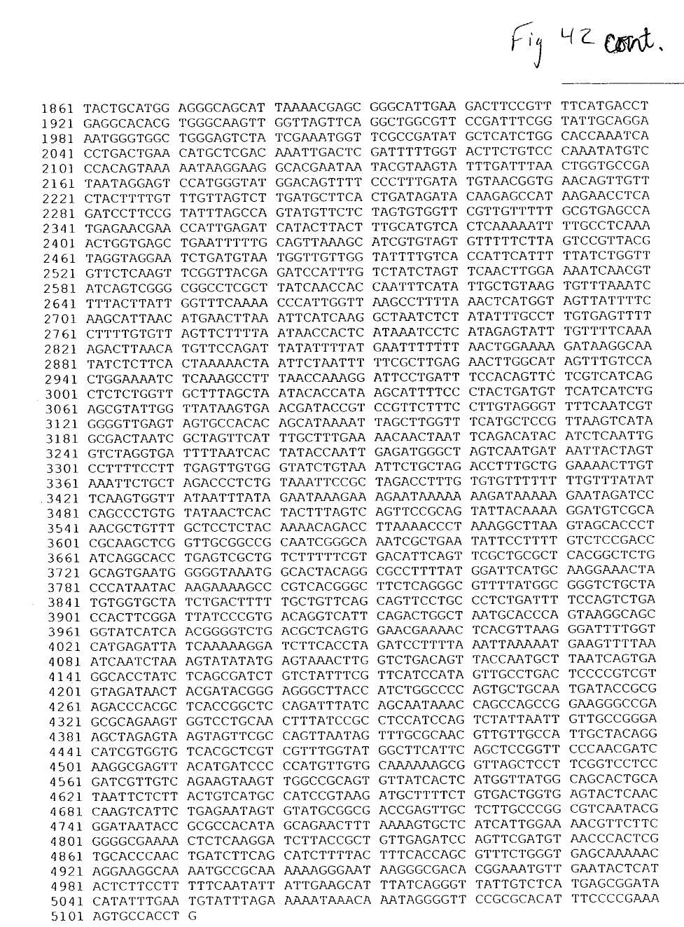

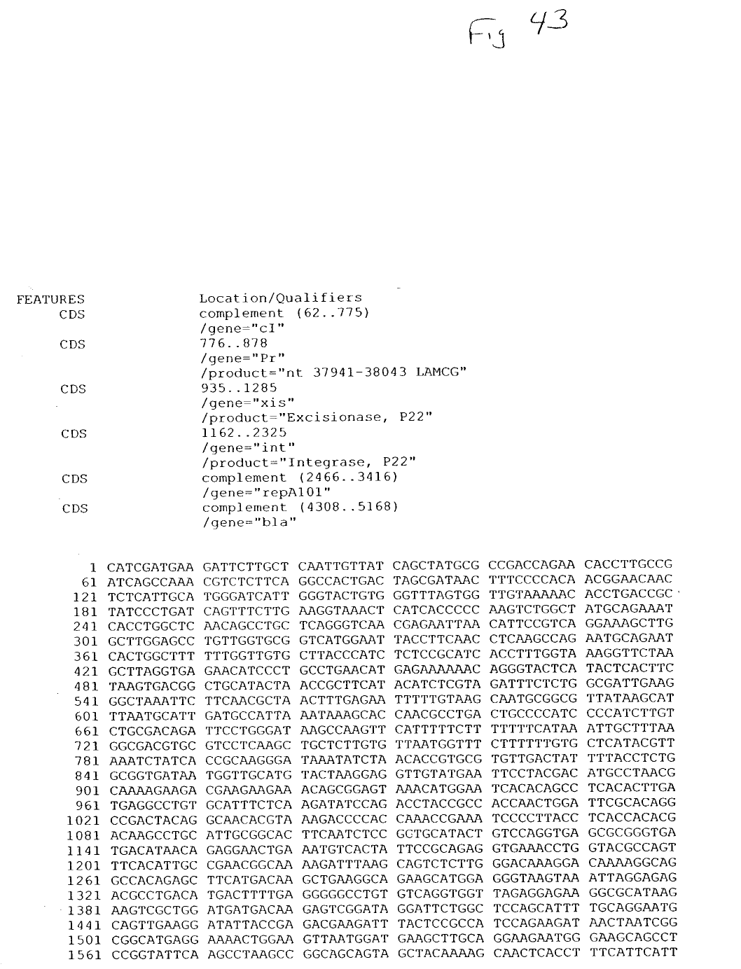

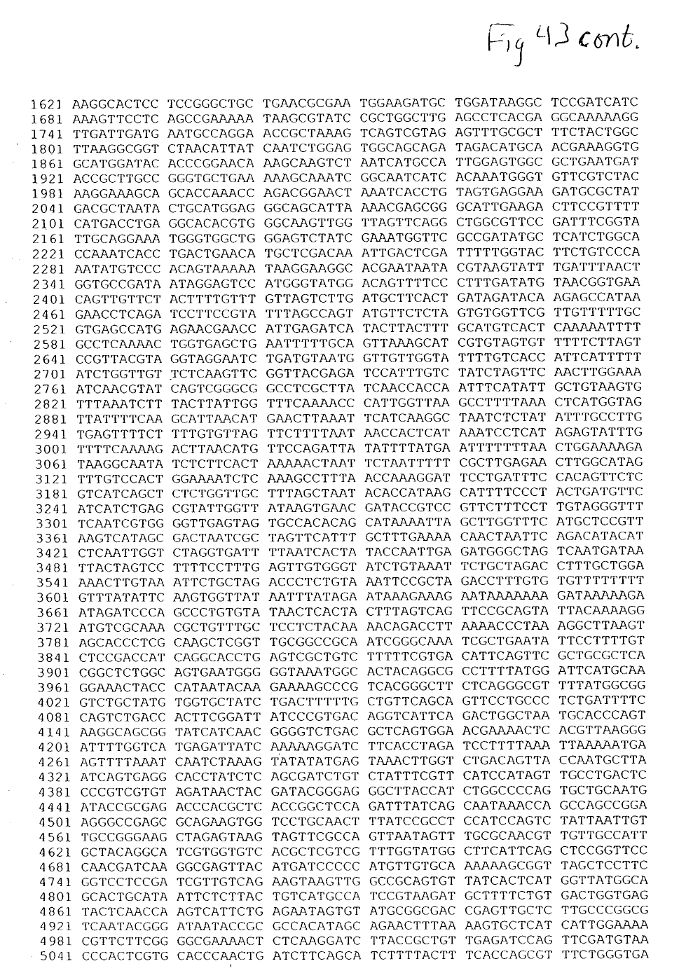

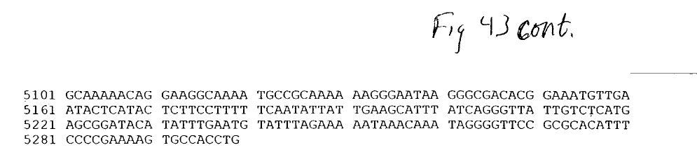

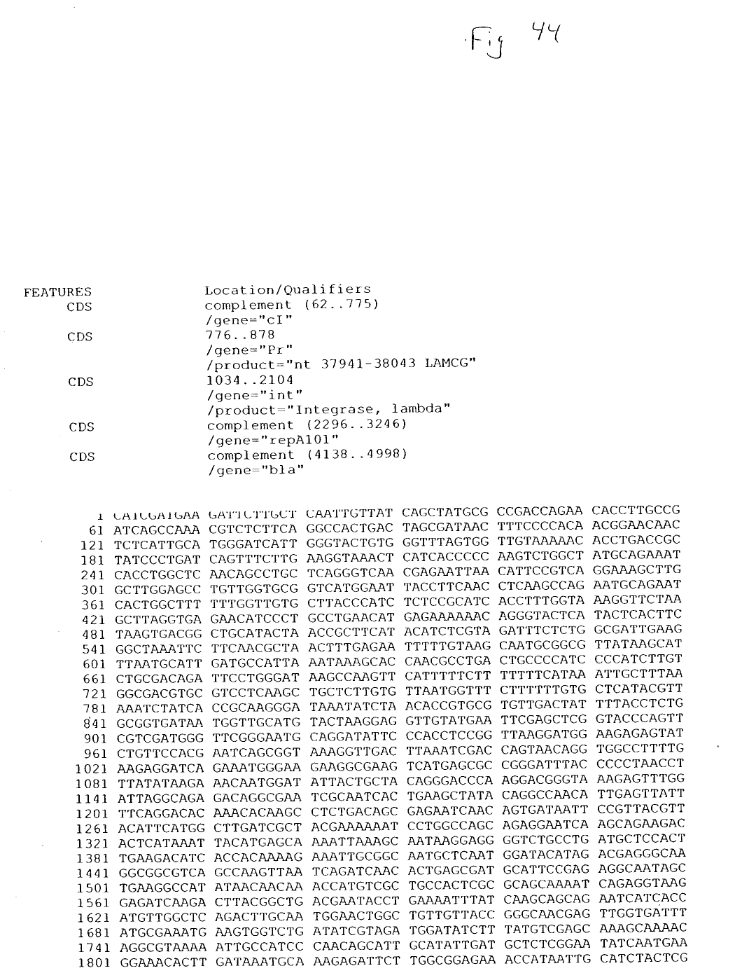

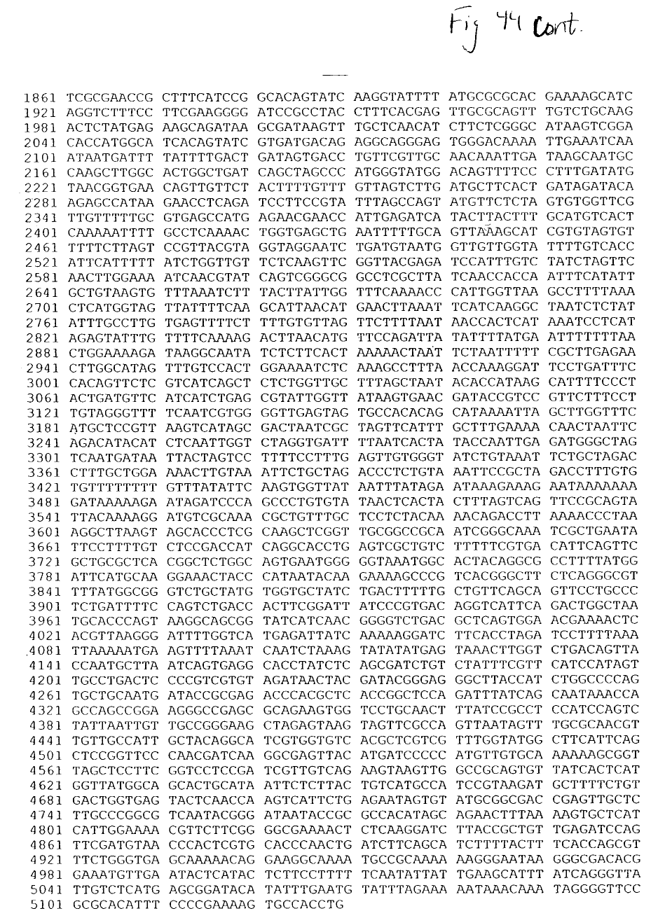

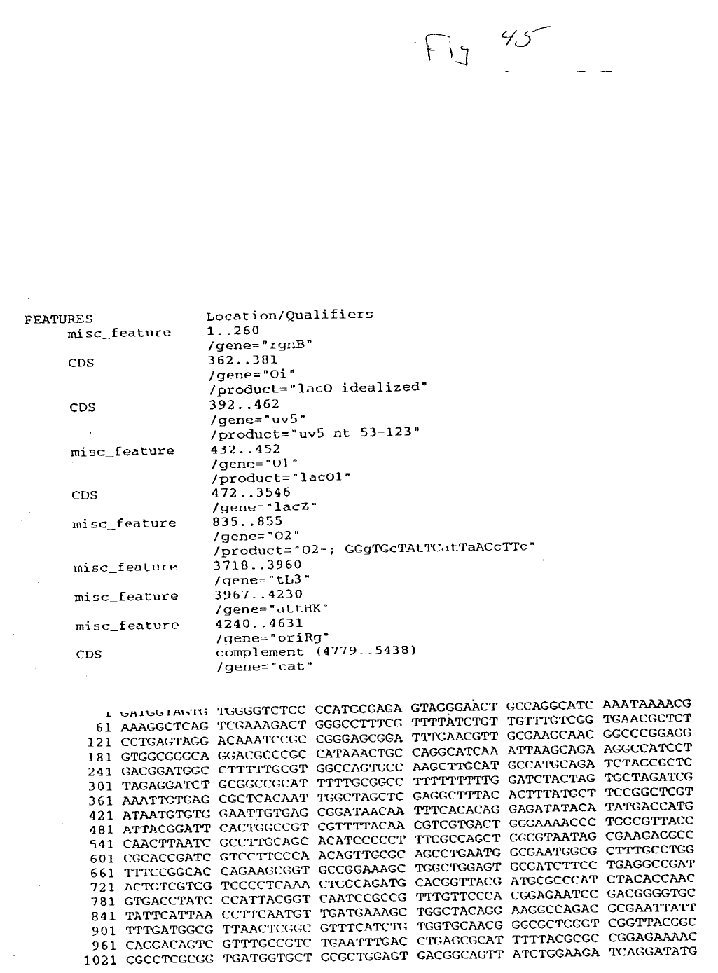

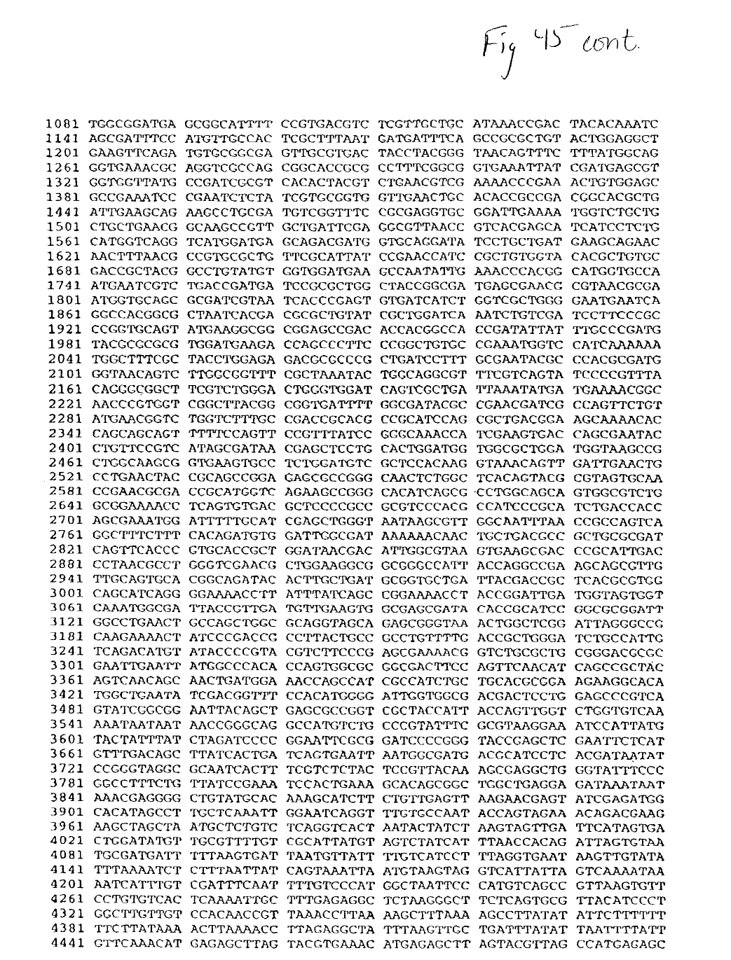

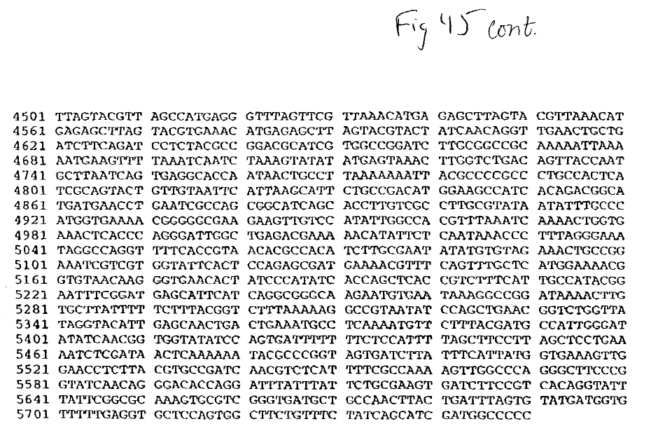

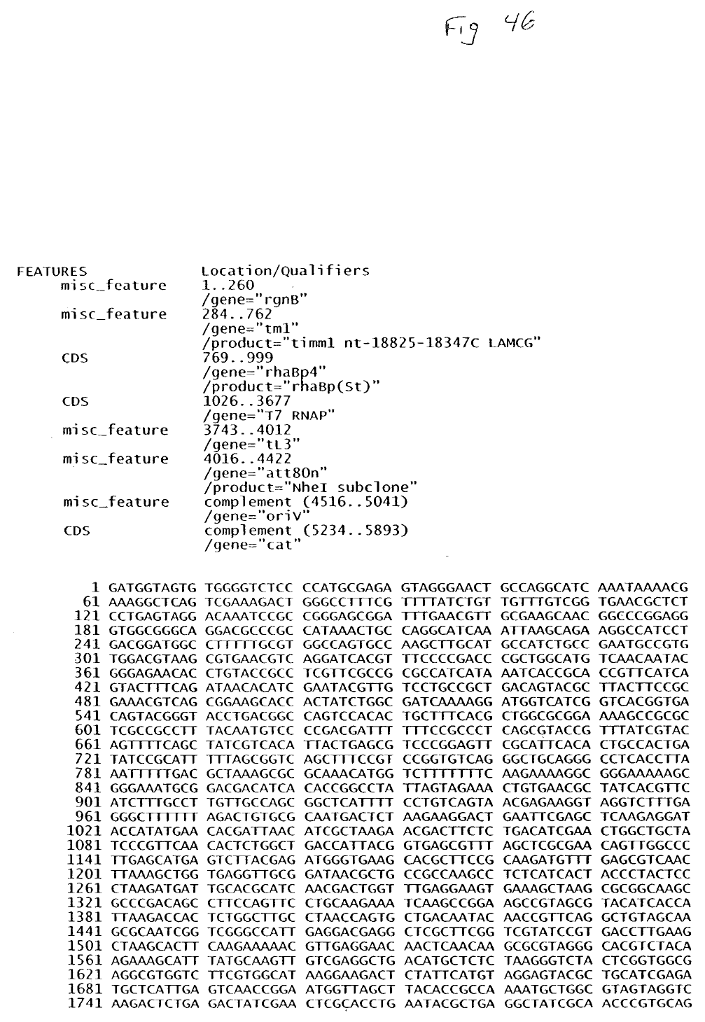

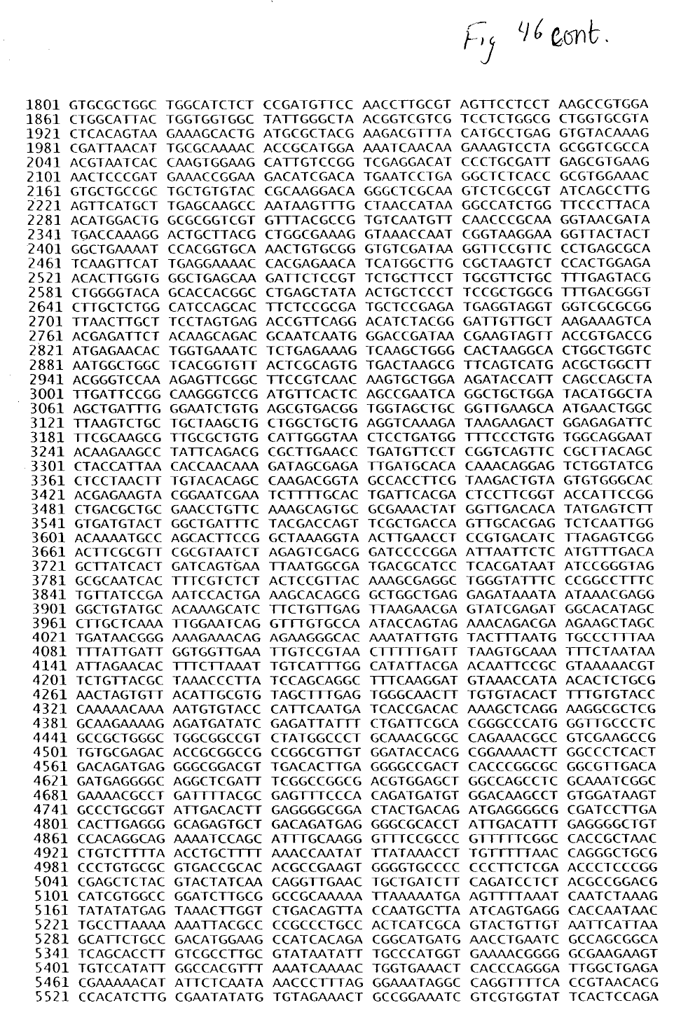

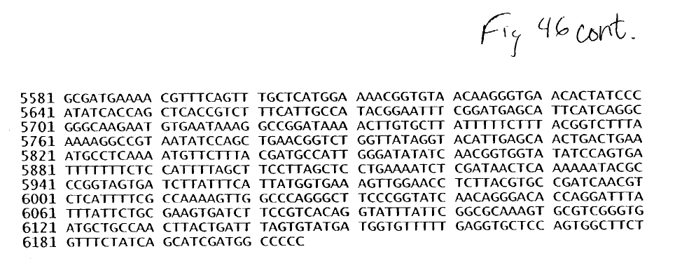

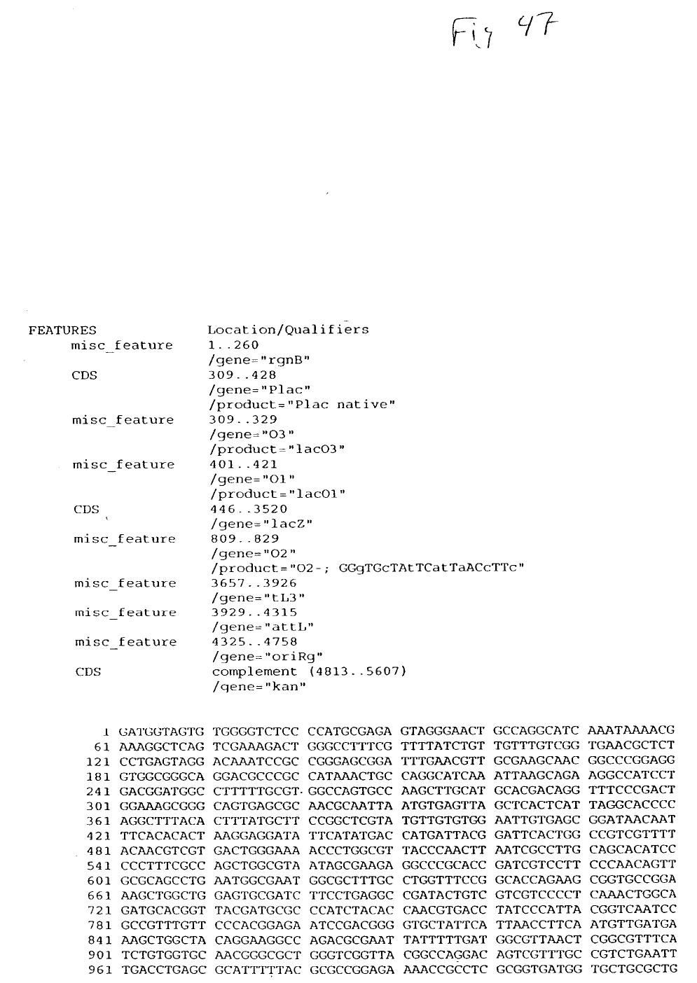

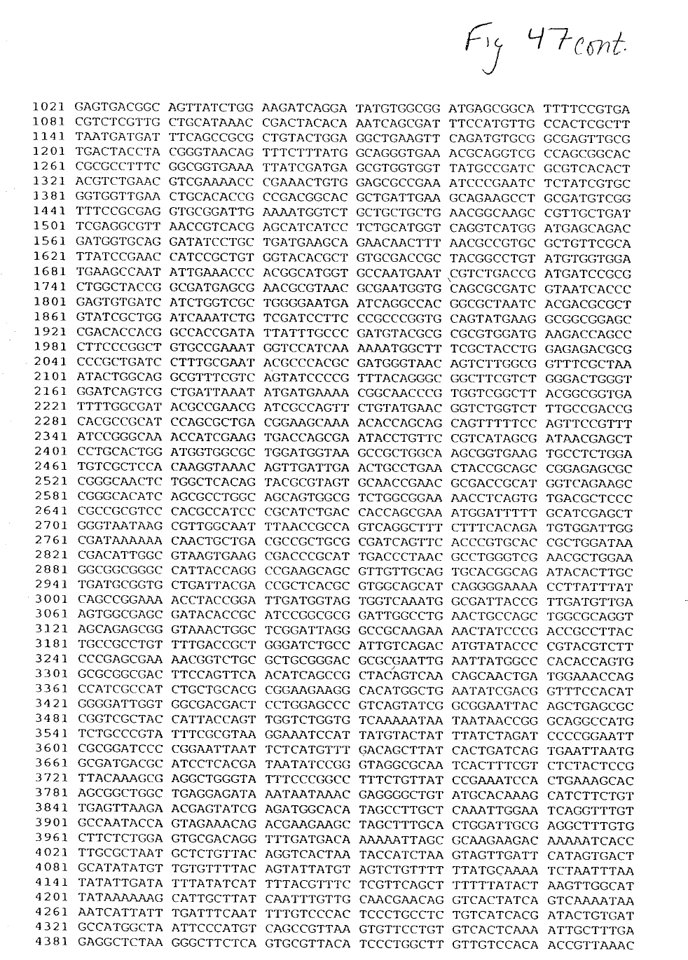

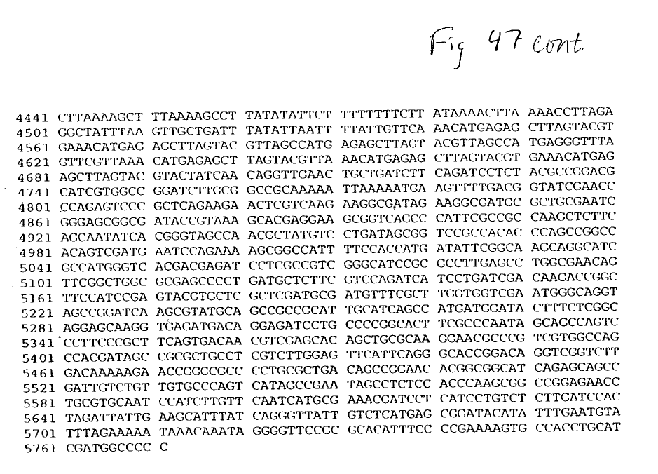

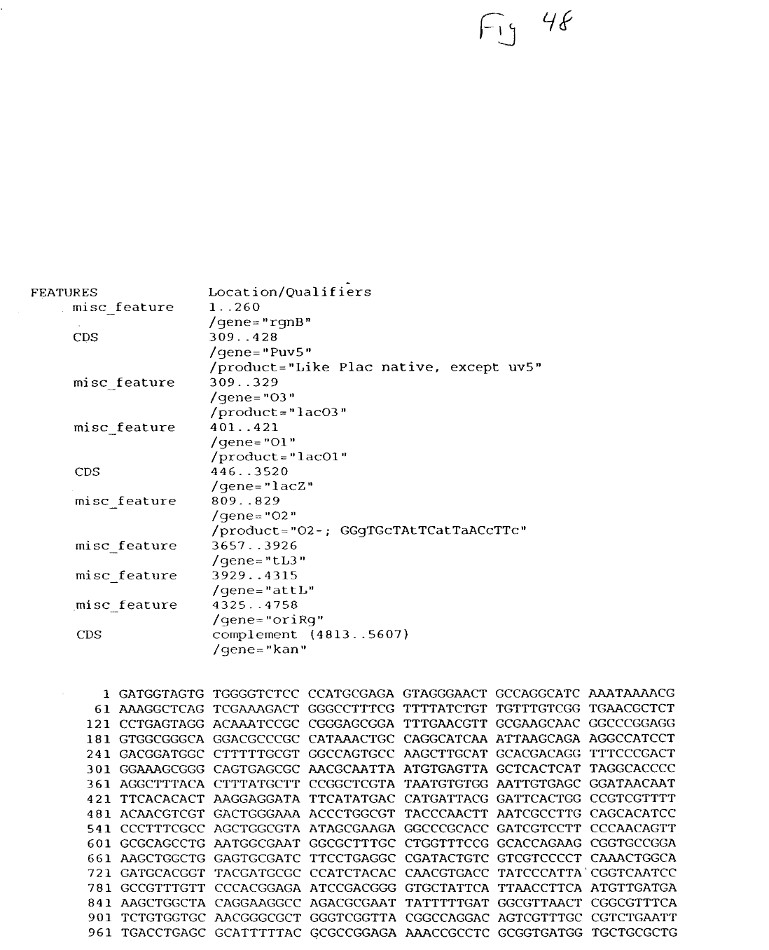

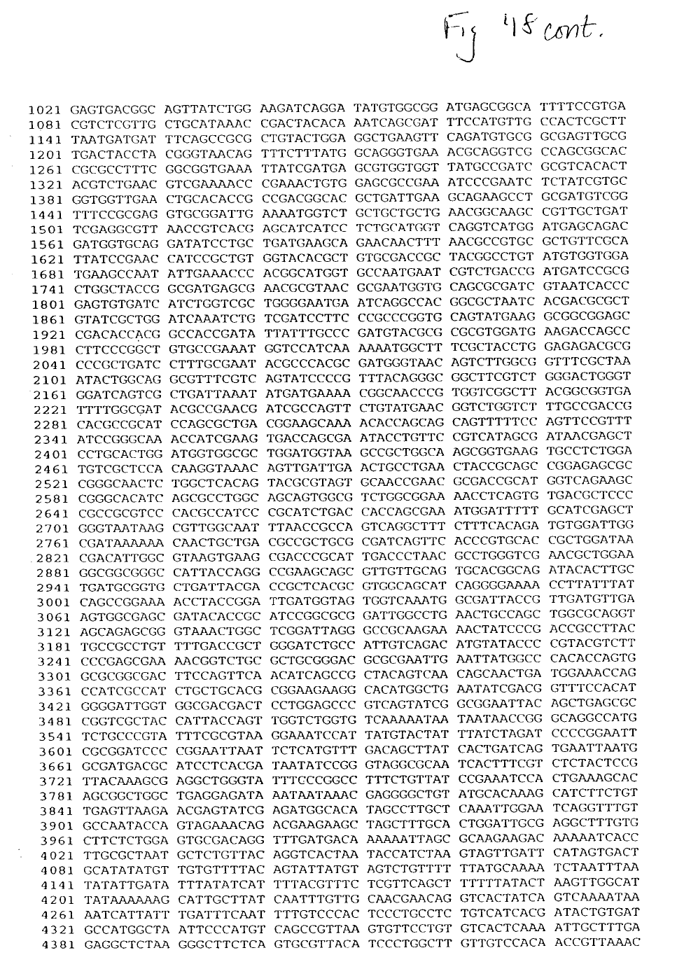

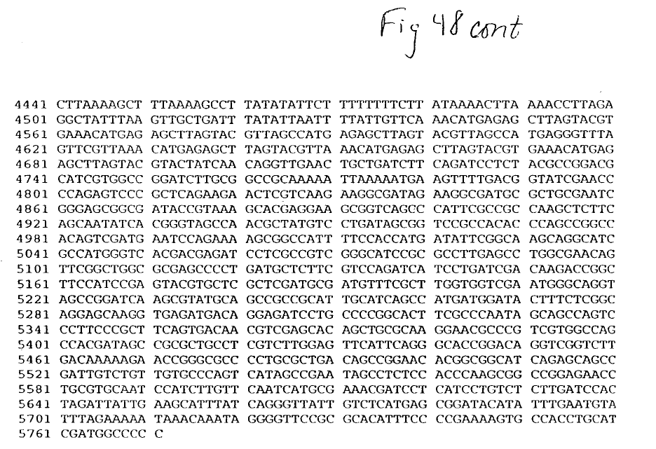

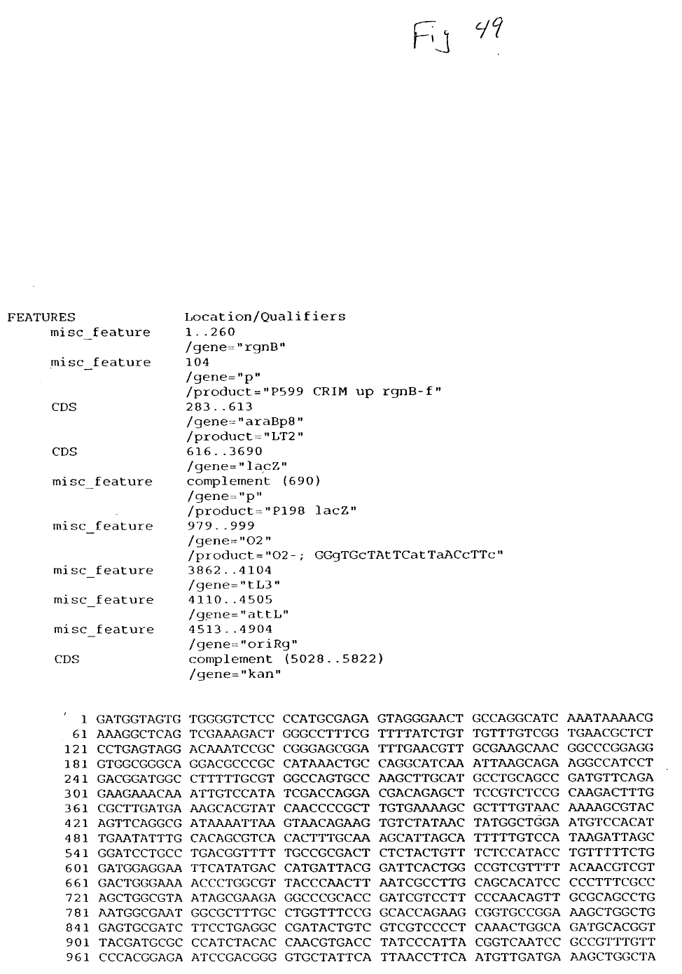

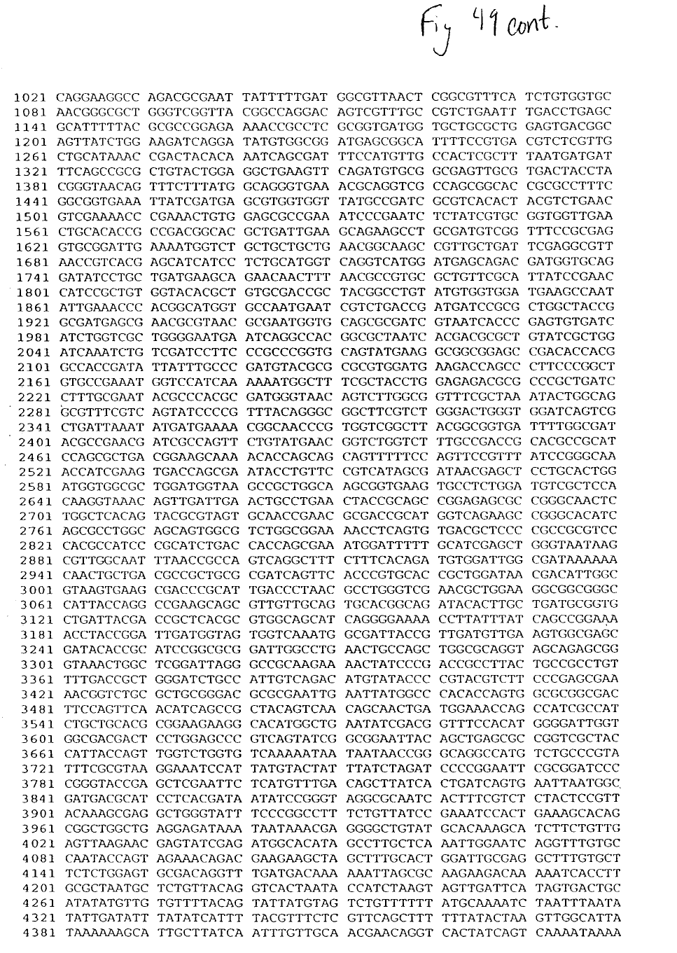

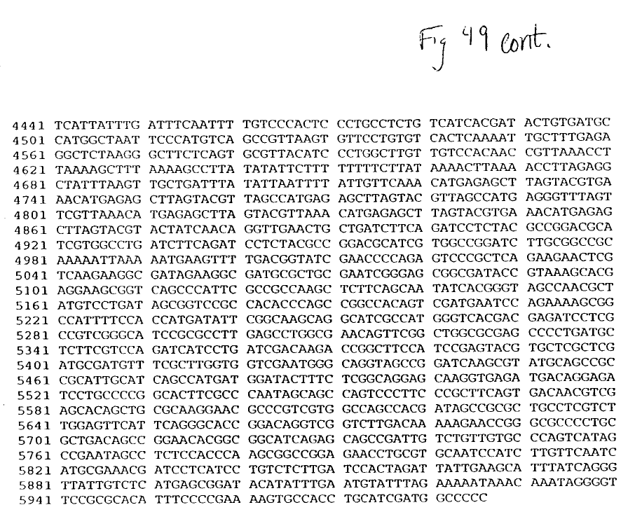

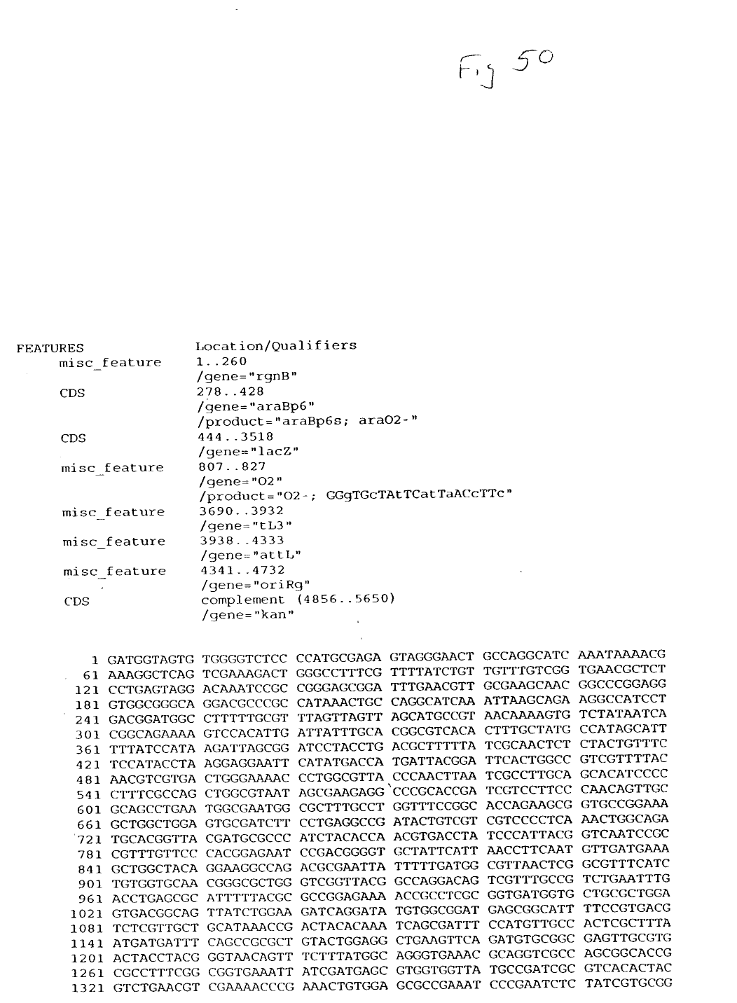

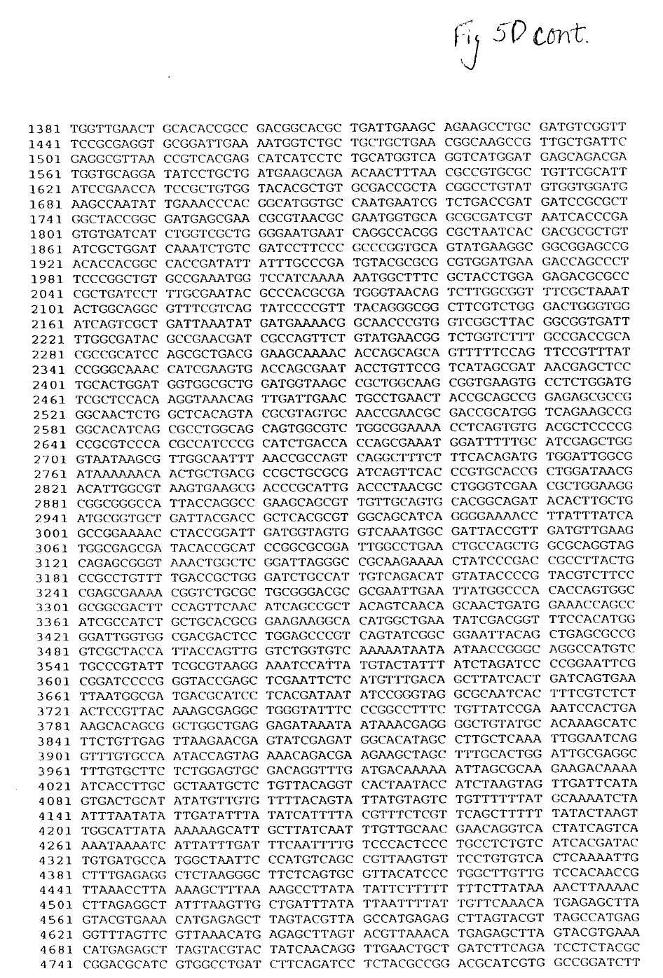

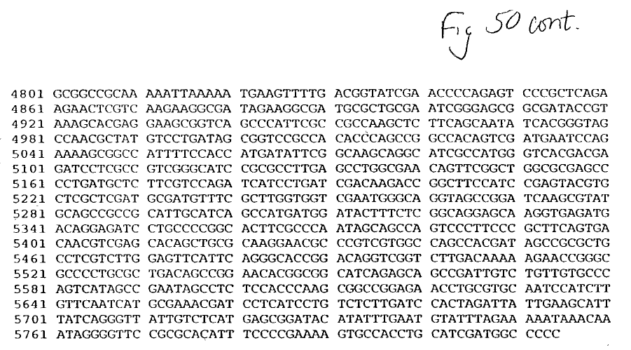

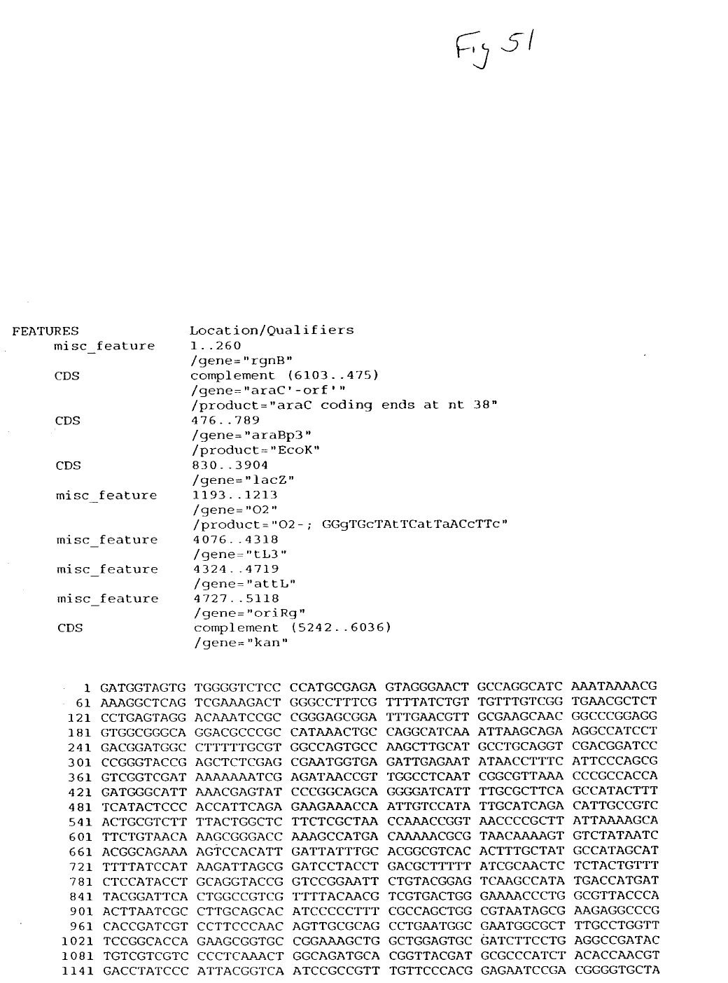

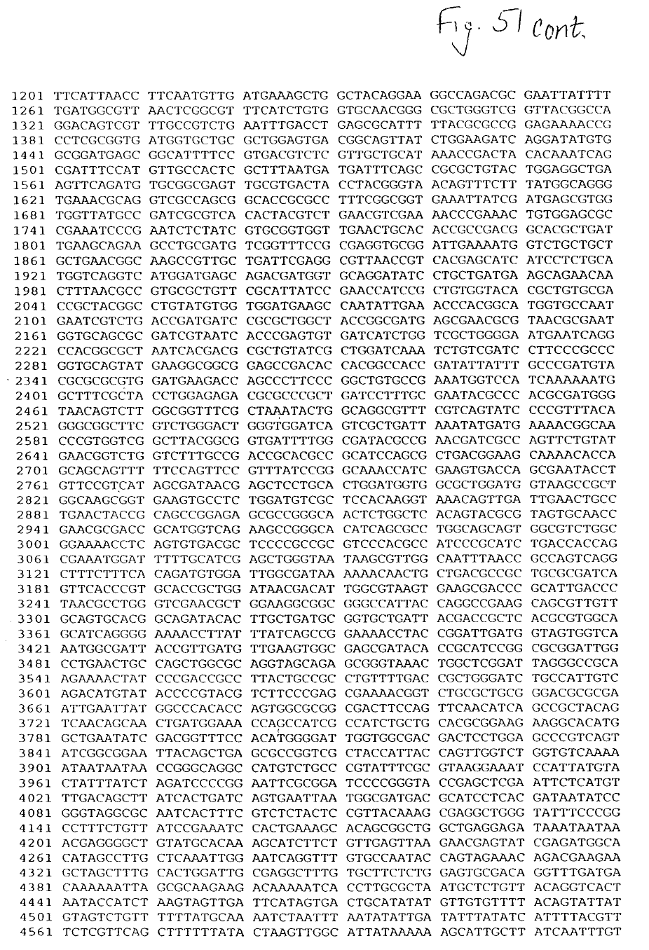

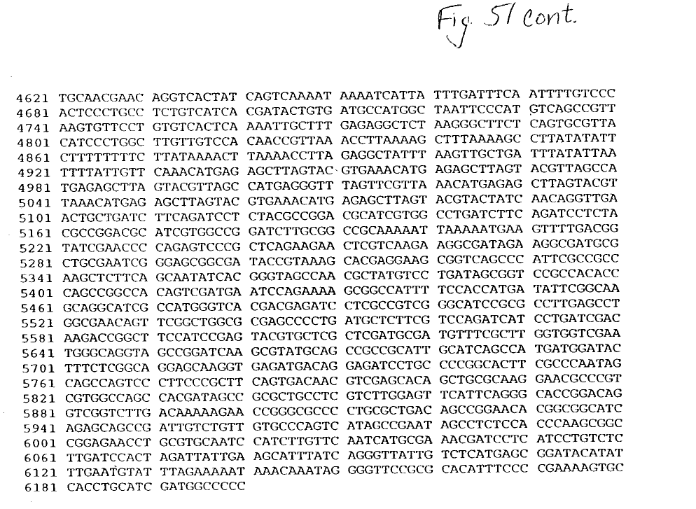

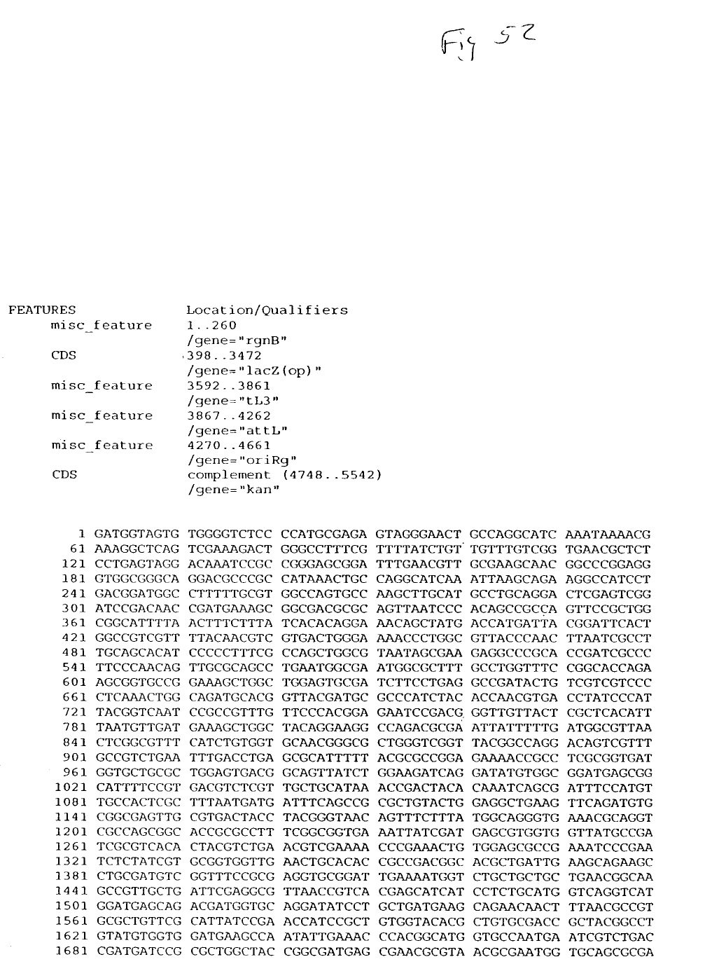

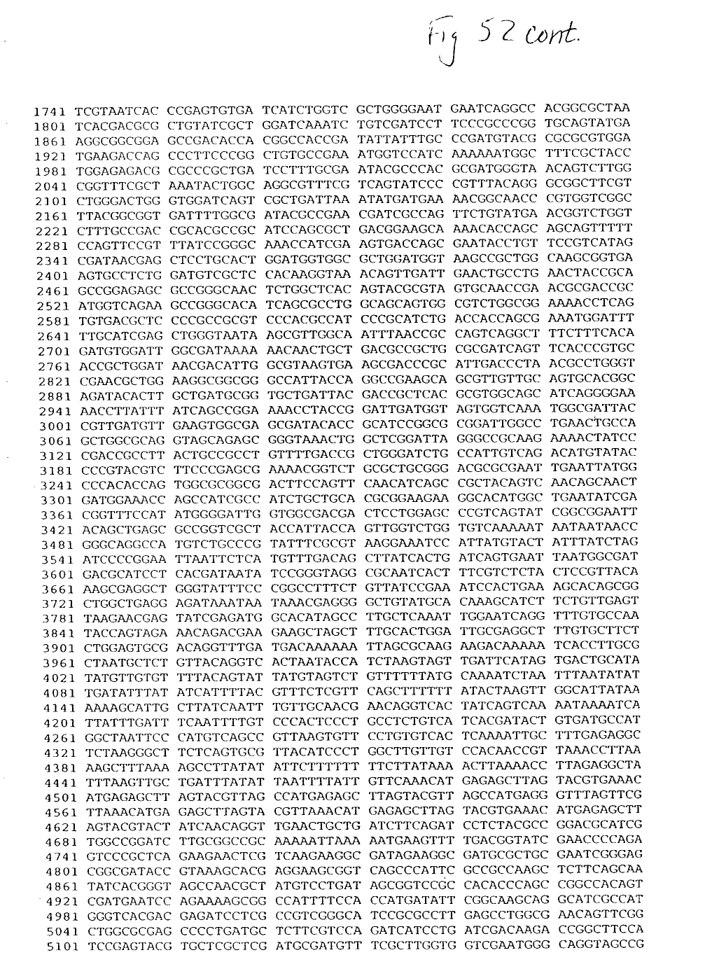

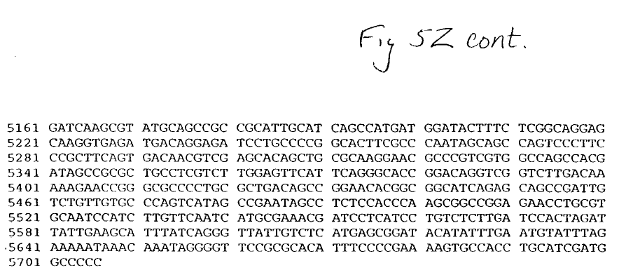

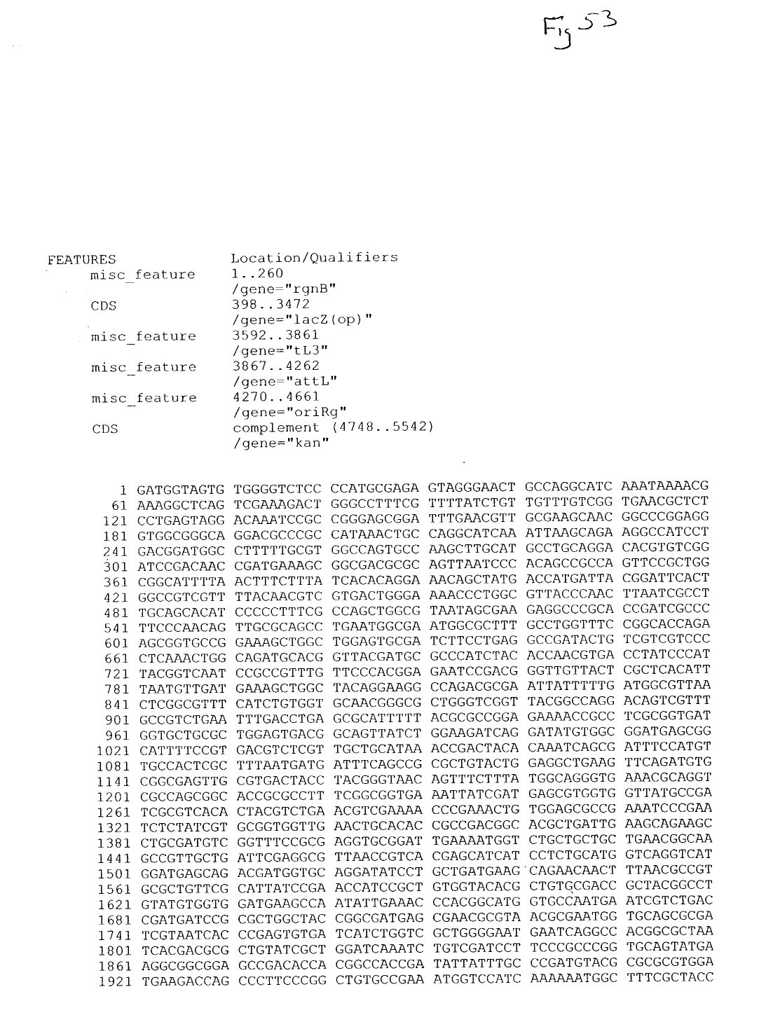

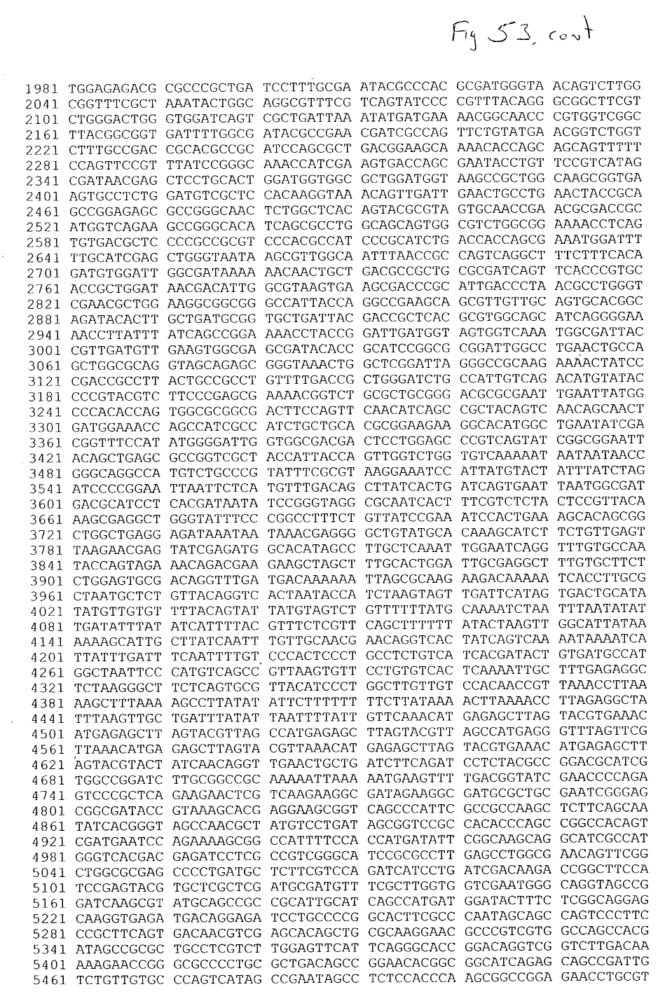

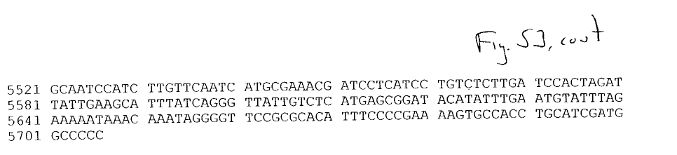

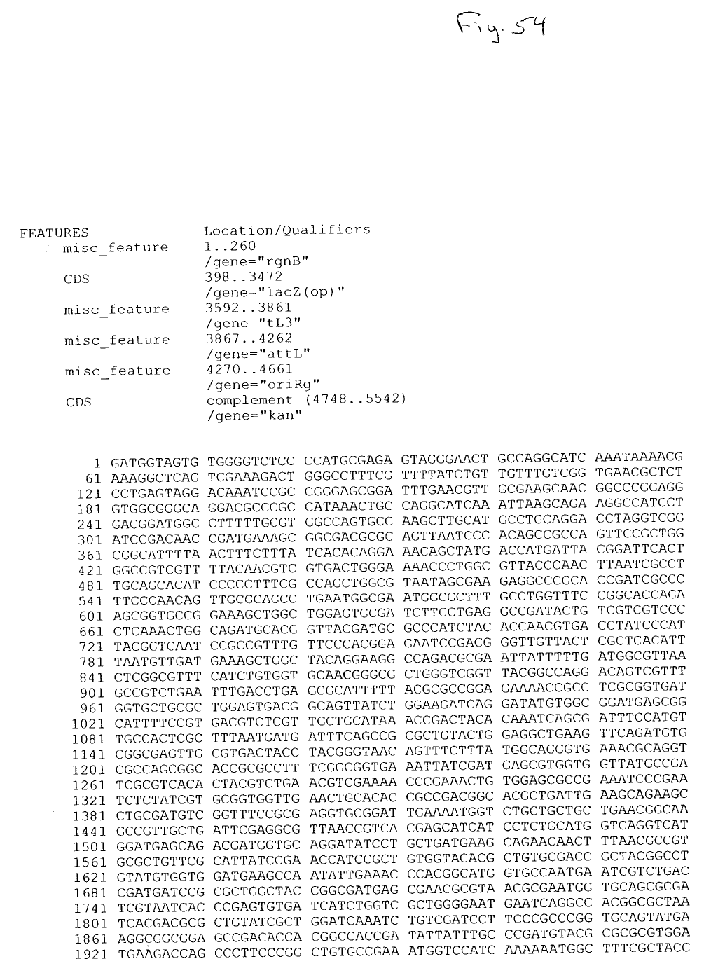

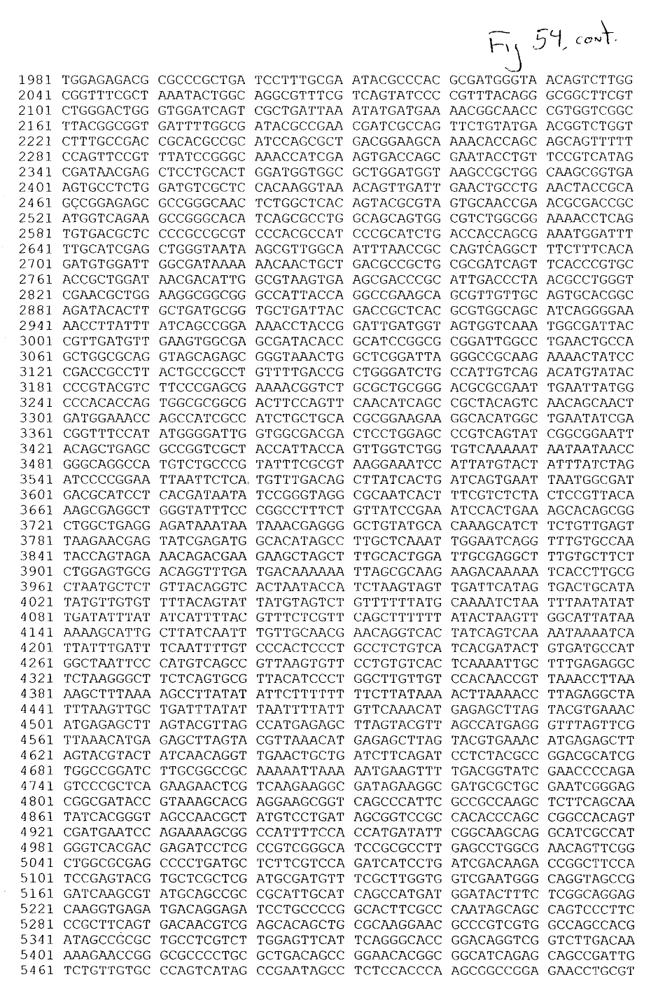

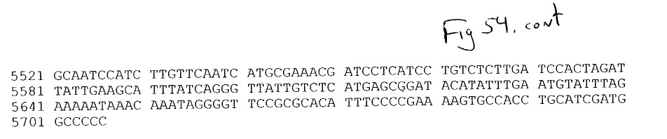

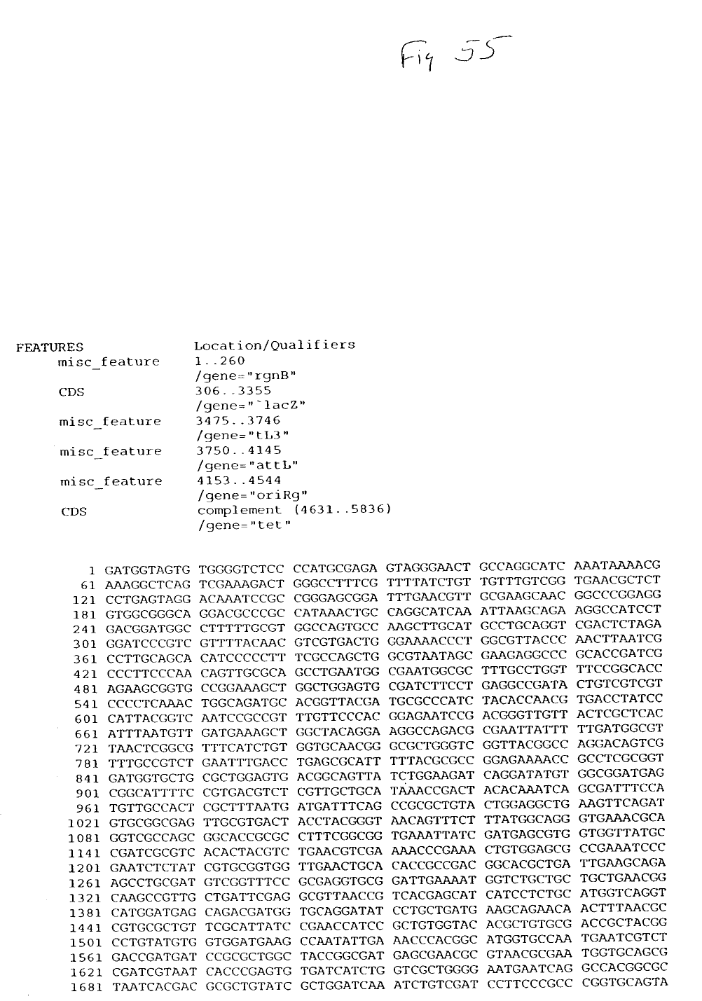

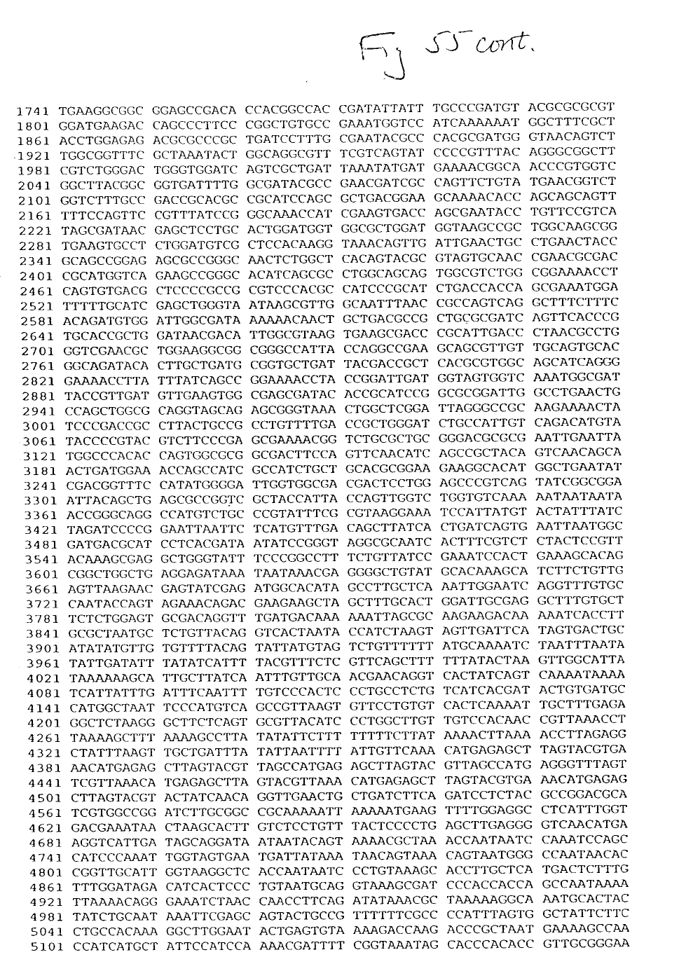

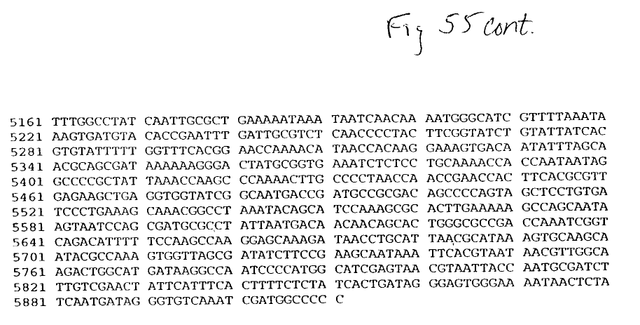

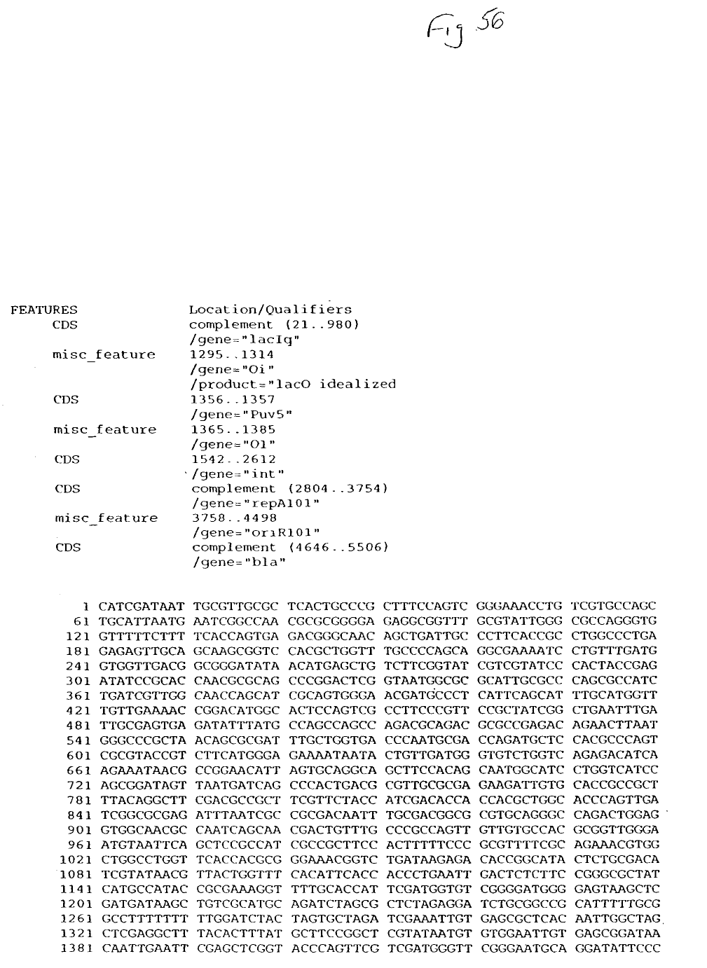

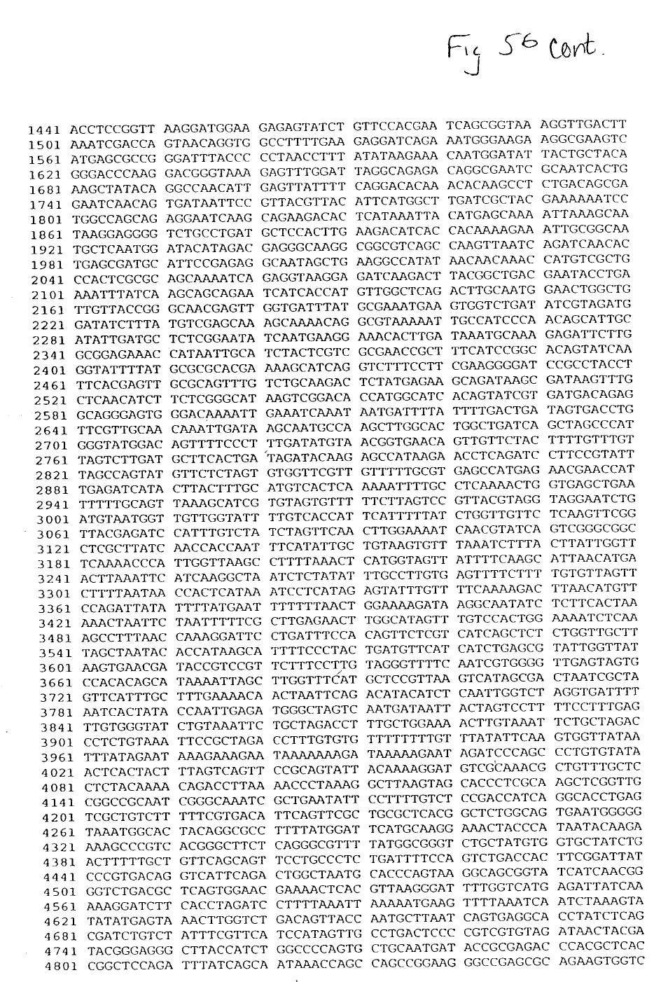

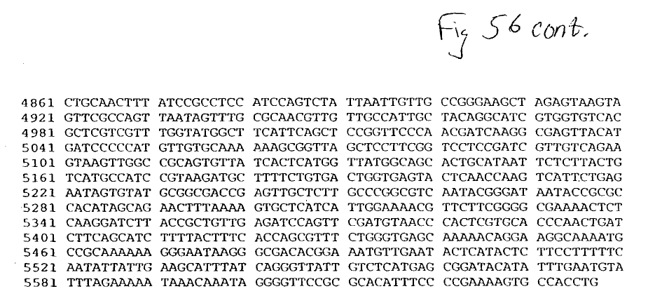

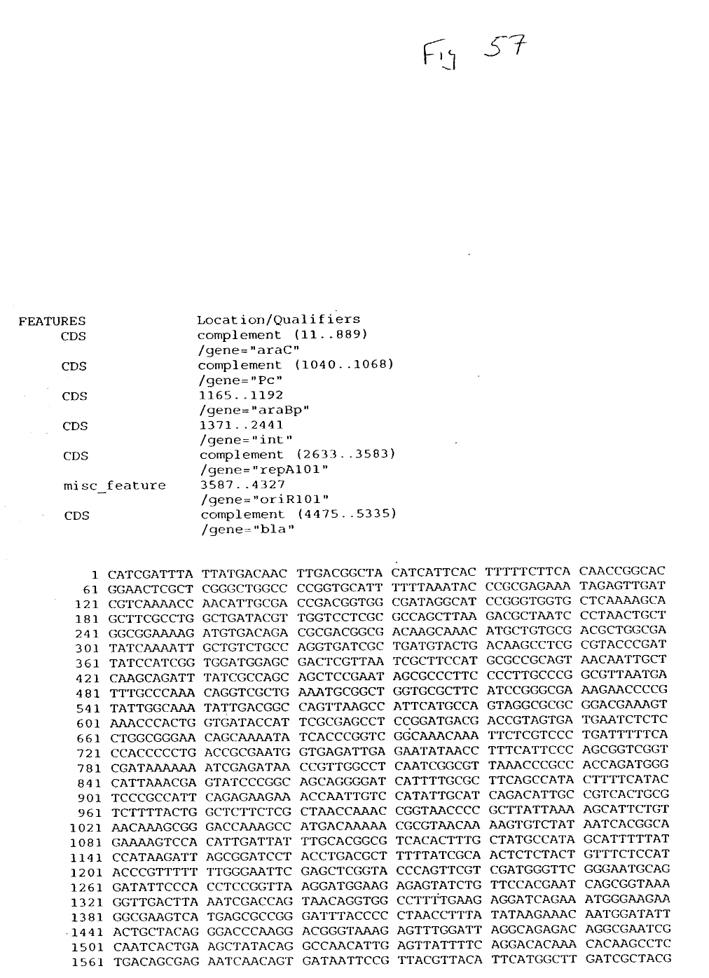

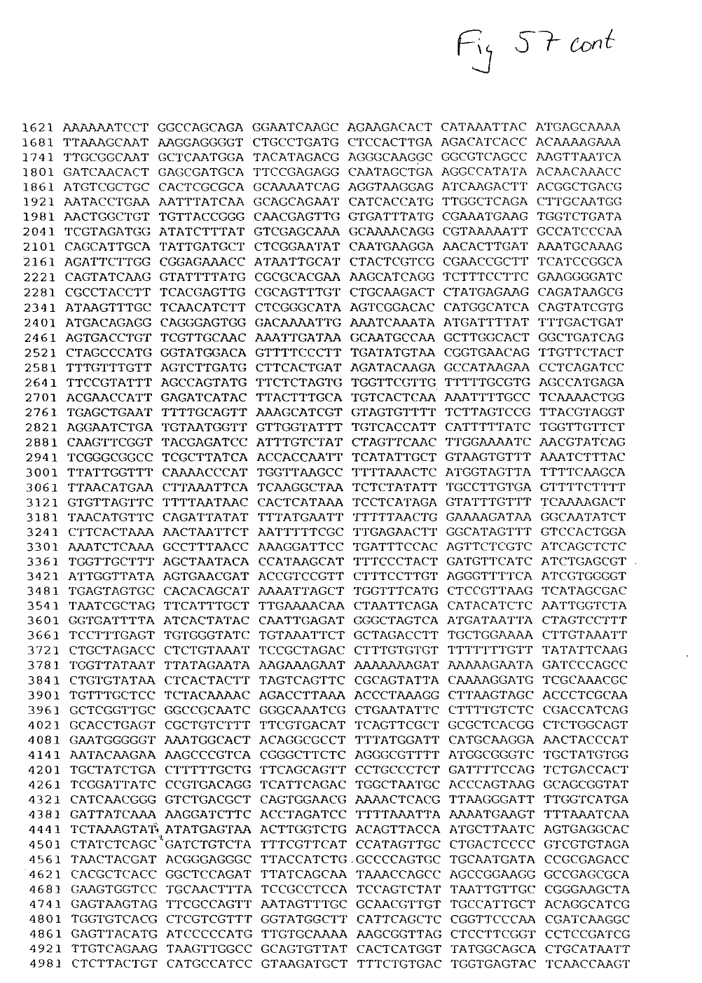

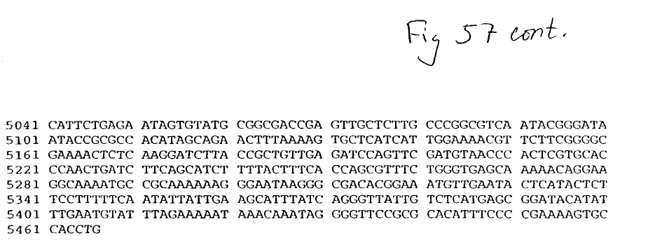

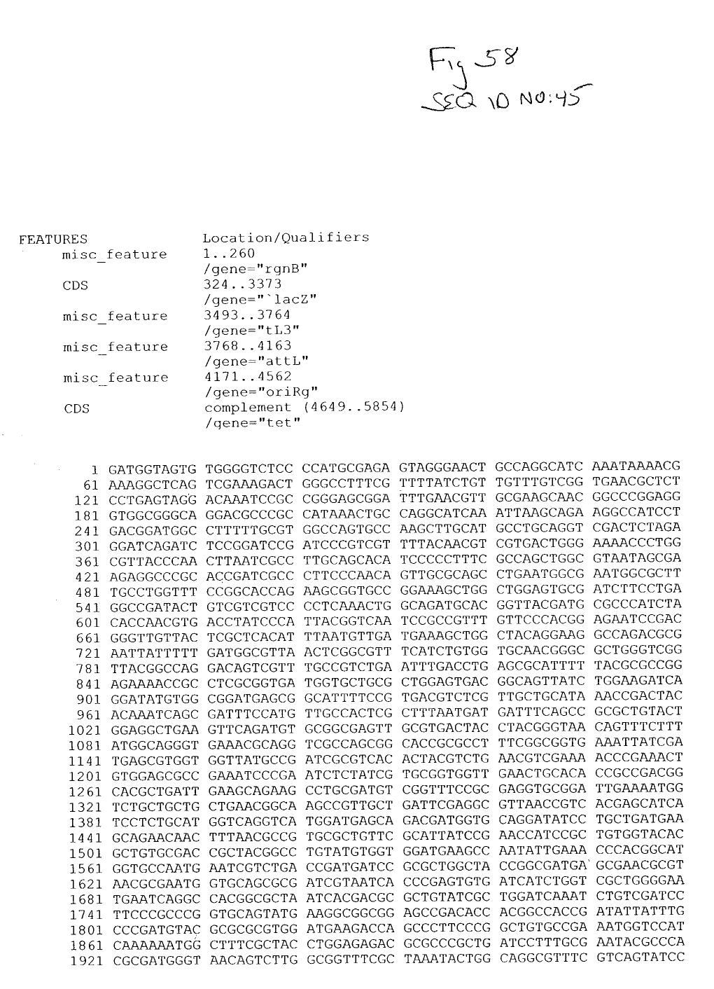

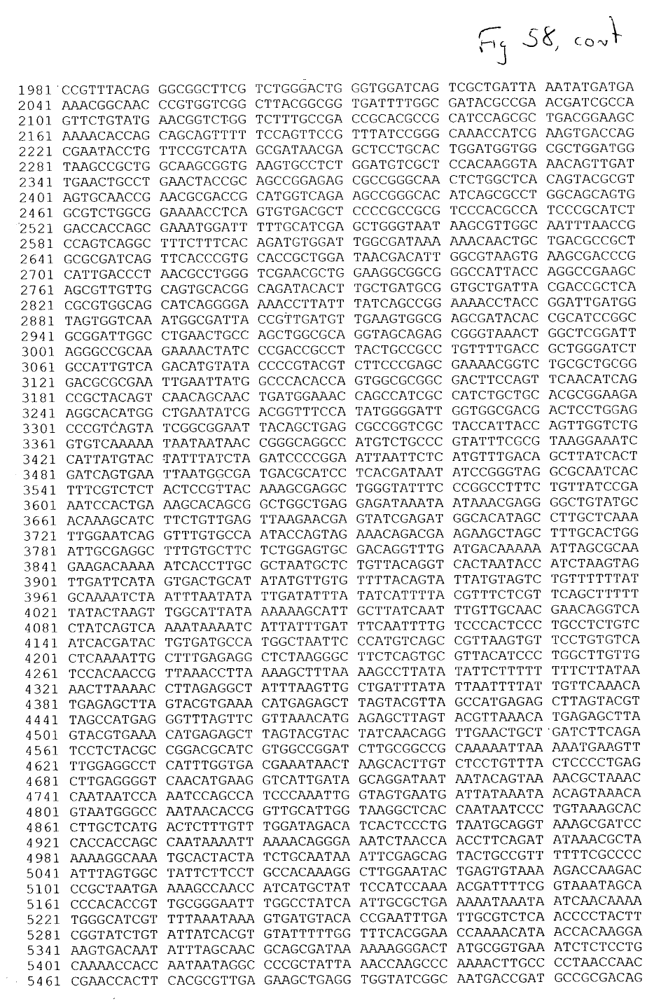

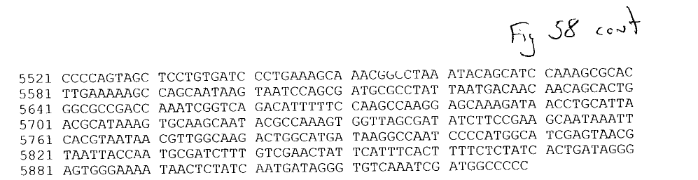

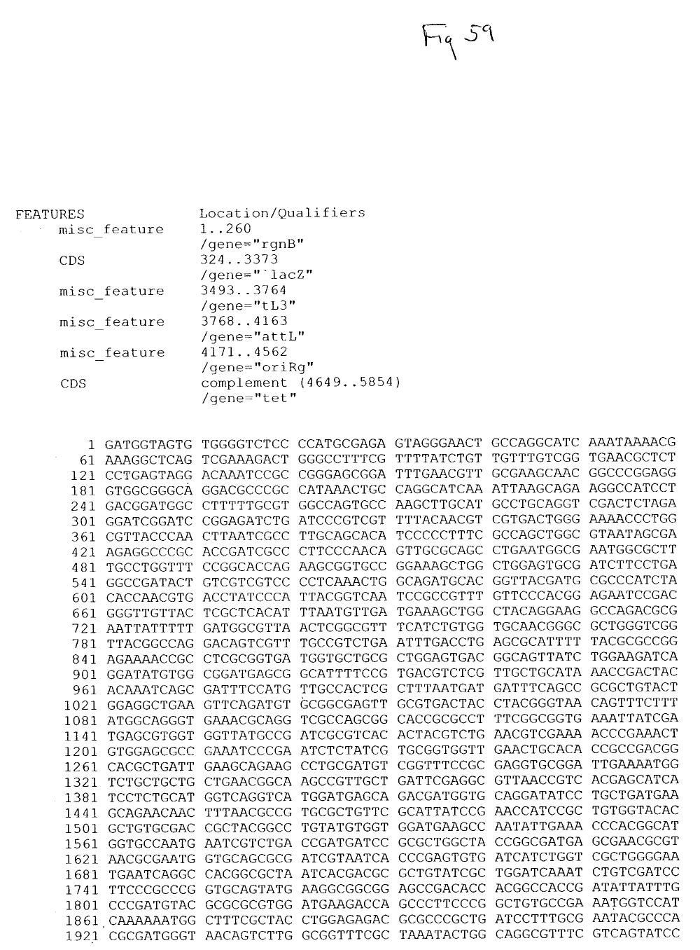

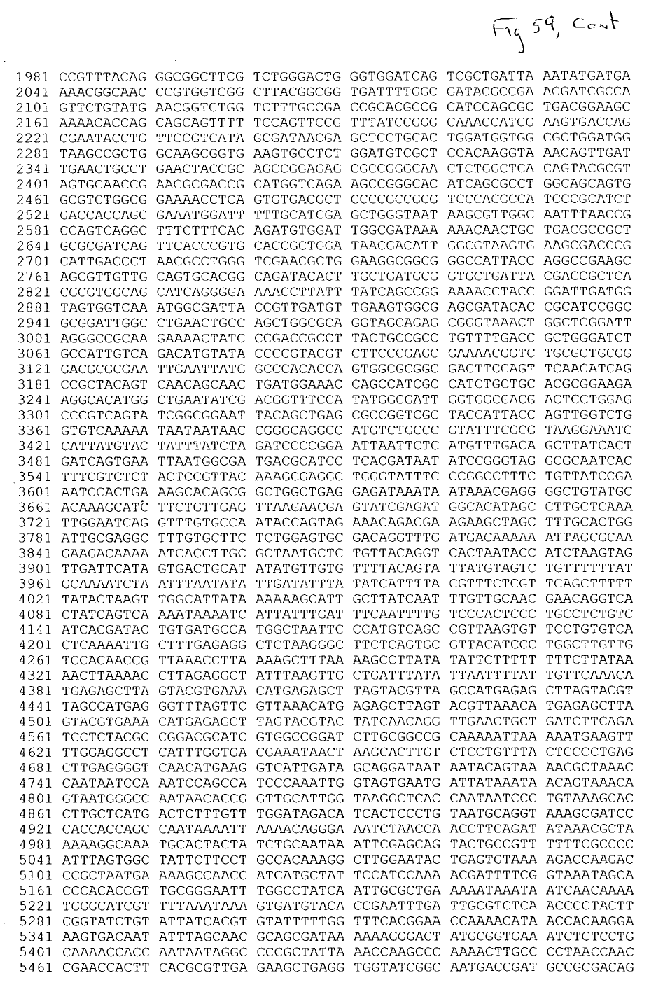

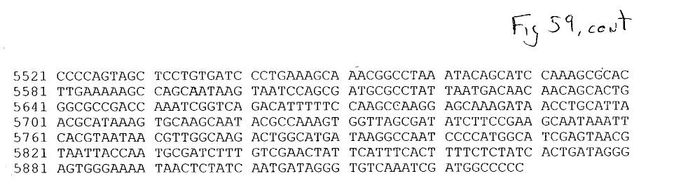

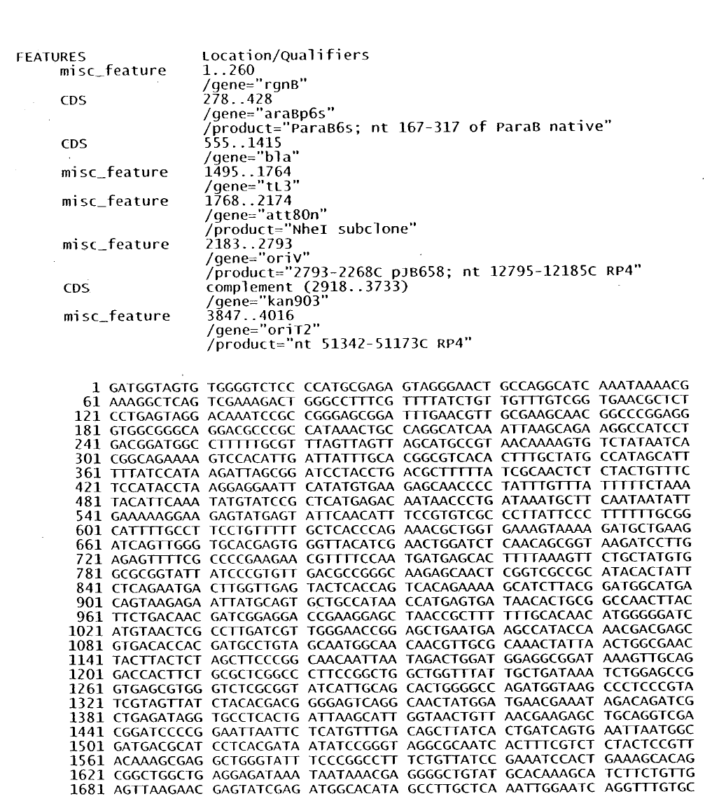

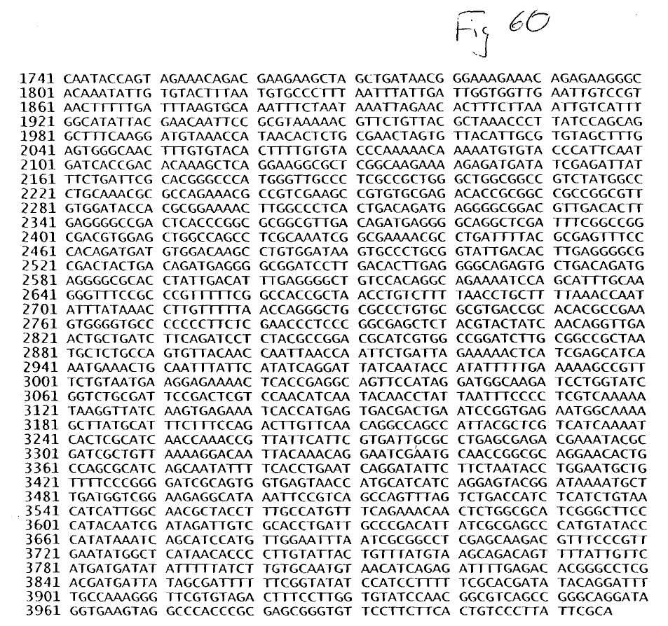

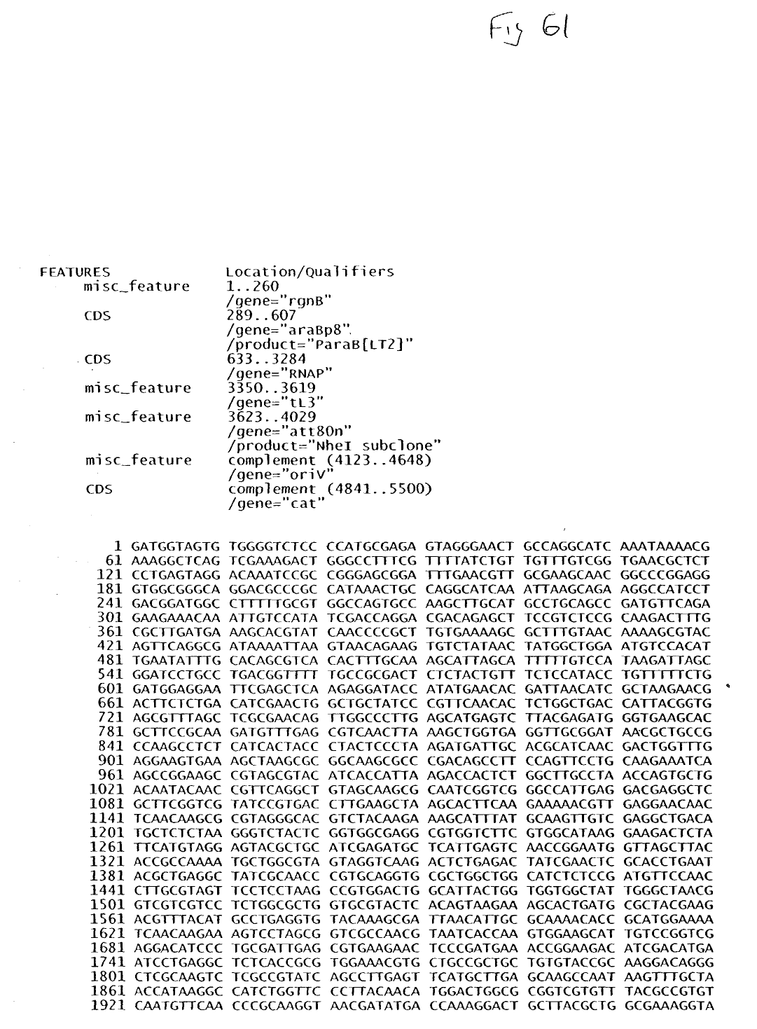

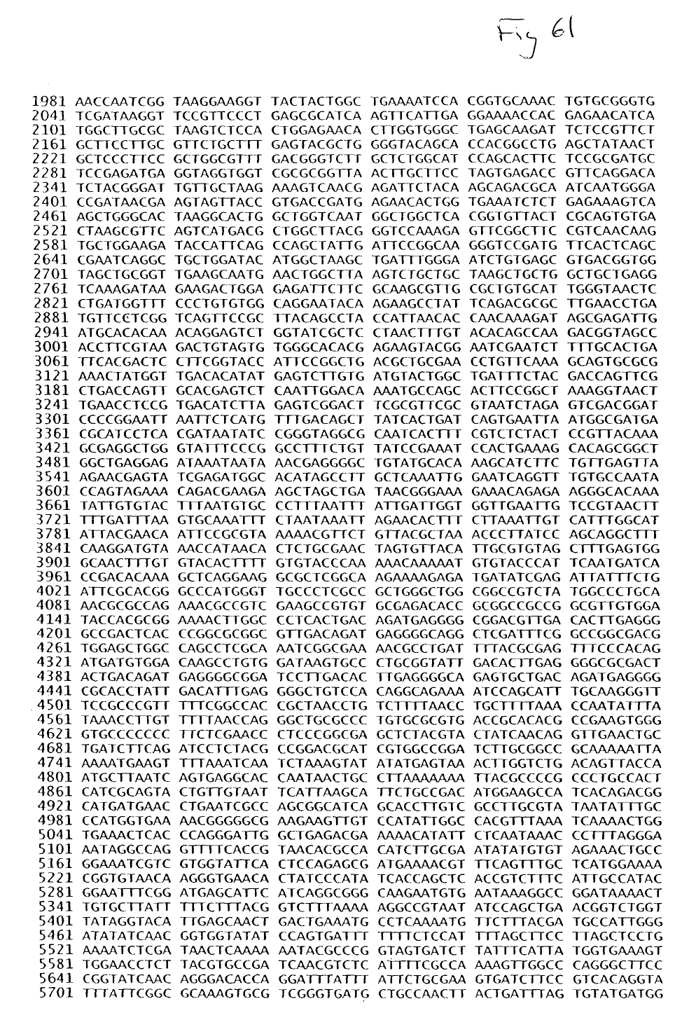

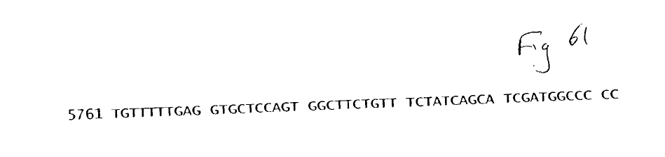

In genetic selections it is generally more useful to use indicator media requiring both LacZ and LacY functions such as lactose MacConkey and lactose tetrazolium. On these media, color differences result from acid production (MacConkey) or redox chemistry (tetrazolium). The preparation and use of these and other Lac indicator media are described elsewhere (Miller J H (1972) Experiments in Molecular Genetics, Cold Spring Harbor Laboratory, Cold Spring Harbor, N.Y.); (Miller J H (1992) A short course in bacterial genetics: a laboratory manual and handbook for Escherichia coli and related bacteria, Cold Spring Harbor Laboratory Press, Plainview). Importantly, unlike media containing X-gal, these can be used to differentiate mutants showing relatively small (10-fold or less) differences in lac expression levels. To facilitate the use of lacZ reporter CRIM plasmids in genetic selections requiring LacY function, we use ΔlacZ mutants in which lacY is induced by IPTG under LacI control (FIG. 13). The Example describes several lacZ reporter CRIM plasmids for easy construction of specific or random lacZ fusions. These have been especially useful not only in studying gene expression in E. coli and S. typhimurium, but also in studying genes from diverse bacteria, including Gram-negative and gram-positive cells (Haldimann et al. (2001) J Bacteriol 183:6384-6393); (Haldimann et al. (1998) J Bacteriol 180:1277-1286); (Haldimann et al. (1996) Proc Natl Acad Sci USA 93:14361-14366); (Haldimann et al. (1997) J Bacteriol 179:5903-5913). Indeed, by using E. coli as a surrogate host and CRIM plasmids encoding various regulatory proteins, we have reconstituted functional regulatory circuits from Gram-positive bacteria in E. coli. For example, we used a combination of CRIM plasmids and lacZ reporter fusions to examine two-component regulatory networks governing vancomycin resistance systems from enterococci in E. coli (Haldimann et al. (1997) J Bacteriol 179:5903-5913); (Silva et al. (1998) Proc Natl Acad Sci USA 95:11951-11956). Furthermore, we extended these studies by isolating and characterizing mutants of the respective regulatory genes by using lacZ fusions similar to those described here.

It should be straight forward to extend these lacZ and other CRIM plasmids to other bacteria. We already showed that attP22 and attλ CRIM plasmids integrate into the appropriate attB sites of S. typhimurium (Haldimann et al. (2001) J Bacteriol 183:6384-6393). It should therefore be feasible to use CRIM plasmids not only to construct specific lacZ fusions to examine genetic regulatory mechanisms in S. typhimurium, but also to make random fusion libraries to screen in S. typhimurium. Once fusions are found, the respective lacZ fusion CRIM plasmid could then be retrieved essentially as done in E. coli. This could be done by using a S. typhimurium galE mutant, which unlike wild-type S. typhimurium is sensitive to phage P1kc (Mojica (1975) Mol Gen Genet 138:113-126). Alternatively, one could use phage P22 transduction in much the same way by using a recipient with the respective CRIM helper plasmid that is either a pir⁺ S. typhimurium or a pir⁺ E. coli carrying a cosmid conferring P22 sensitivity (Neal et al. J Bacteriol 175:7115-7118).

Several F′ factors carrying various attB sites also exist (Low (1972) Bacteriol Rev 36:587-607). By integrating lacZ fusion CRIM plasmids into an appropriate F′ factor, it would be simple to transfer the fusion into other hosts by bacterial conjugation, a much more efficient process than phage transduction or DNA transformation. These could also be used to introduce lacZ fusion CRIM plasmids into closely related bacteria that may lack particular attB sites. Also, unlike F′ factors carrying lacZ fusion phages, F′ factors harboring lacZ reporter CRIM plasmids would not undergo zygotic induction upon conjugal transfer. Alternatively, low-copy-number, broad-host-range plasmids with an attB site(s) could be engineered. To do this, it would be especially advantageous to use a plasmid(s) that can also be transferred by conjugation. Studies using such approaches with lacZ reporter CRIM plasmids to extend CRIM technology to diverse bacteria are underway. In conjunction with efficient means for gene disruption (Datsenko et al. (2000) Proc Natl Acad Sci USA 97:6640-6645), lacZ reporter and other CRIM plasmids (Haldimann et al. (2001) J Bacteriol 183:6384-6393) provide new tools for gene analysis on a genome-wide scale.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A kit for integrating a polynucleotide into genomic DNA of a microbe, the kit comprising:
a first plasmid selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:32, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:45, SEQ ID NO:46, and a combination thereof; and
a second plasmid selected from the group consisting of SEQ ID NO:23, SEQ ID NO:25, SEQ ID NO:27, SEQ ID NO:29, SEQ ID NO:31, SEQ ID NO:43, SEQ ID NO:44, and a combination thereof.

2. A kit for integrating a polynucleotide into genomic DNA of a microbe, the kit comprising:
a first plasmid comprising a nucleotide sequence selected from the group consisting of nucleotides about 1-about 2,060 and about 4,155-about 6,664 of SEQ ID NO:1, nucleotides about 1-about 2,060 and about 4,158-about 6,668 of SEQ ID NO:2, nucleotides about 1-about 100 and about 1,135-about 3,663 of SEQ ID NO:3, SEQ ID NO:4, nucleotides about 1-about 100 and about 1,031-about 3,534 of SEQ ID NO:5, the nucleotide sequence SEQ ID NO:6, nucleotides about 1-about 100 and about 1,135-about 3,843 of SEQ ID NO:7, SEQ ID NO:8, nucleotides about 1-about 148 and about 3224-about 5739SEQ ID NO:9, SEQ ID NO:10, SEQ ID NO:11, SEQ ID NO:12, nucleotides about 1-about 547 and about 1,238-about 3,695 of SEQ ID NO:13, nucleotides about 1-about 255 and about 1,551-about 3,653 of SEQ ID NO:14, SEQ ID NO:15, SEQ ID NO:16, nucleotides about 1-about 129 and about 1,426-about 3,527 of SEQ ID NO:17, SEQ ID NO:18, nucleotides about 1-about 2,060 and about 4,157-about 6,742 of SEQ ID NO:19, nucleotides about 1-about 100 and about 2,195-about 4,782 of SEQ ID NO:20, nucleotides about 1-about 332 and about 3,408-about 5,948 of SEQ ID NO:21, nucleotides about 1-about 471 and about 3547-about 5750 of SEQ ID NO:32, SEQ ID NO:33, nucleotides about 1-about 445 and about 3521-about 5771 of SEQ ID NO:34, nucleotides about 1-about 445 and about 3521-about 5771 of SEQ ID NO:35, nucleotides about 1-about 615 and about 3691-about 5989 of SEQ ID NO:36, nucleotides about 1-about 443 and about 3519-about 5814 of SEQ ID NO:37, nucleotides about 1-about 829 and about 3905-about 6200 of SEQ ID NO:38, nucleotides about 1-about 397 and about 3473-about 5706 of SEQ ID NO:39, SEQ ID NO:40, SEQ ID NO:41, SEQ ID NO:42, SEQ ID NO:45, SEQ ID NO:46, and a combination thereof; and
a second plasmid, wherein the second plasmid comprises a nucleotide sequence encoding an integrase polypeptide, wherein the nucleotide sequence has at least about 80% identity to a nucleotide sequence selected from the group consisting of nucleotides 908-1981 of SEQ ID NO:23, nucleotides 937-2079 of SEQ ID NO:25, nucleotides 967-2217 of SEQ ID NO:27, nucleotides 974-2137 of SEQ ID NO:29, nucleotides 1034-2104 of SEQ ID NO:31, nucleotides 1542-2612 of SEQ ID NO:43, nucleotides 1371-2441 of SEQ ID NO:44, and a combination thereof, and wherein the percent identity is determined by a BLAST2 search algorithm using default parameters.

3. A kit for excising a polynucleotide from genomic DNA of a microbe, the kit comprising:
a first plasmid selected from the group consisting of SEQ ID NO:22, SEQ ID NO:24, SEQ ID NO:26, SEQ ID NO:28, SEQ ID NO:30, and a combination thereof.

4. A method for integrating a polynucleotide into genomic DNA of a microbe, comprising:
introducing a first plasmid to a microbe wherein the plasmid comprises an attP site selected from the group consisting of attλ, attHK022, attP21, attP22, and attφ80, and the first plasmid further comprises a polynucleotide to be integrated into genomic DNA of a microbe, wherein the plasmid does not replicate in the microbe, and wherein the microbe comprises an attB site corresponding to the attP site and a second plasmid comprising a coding sequence encoding an integrase that catalyzes site-specific recombination between the attP site and the attB site; and
detecting the microbe comprising a single copy of the first plasmid integrated into the genomic DNA of the microbe.

5. The method of claim 4 wherein the microbe is a gram-negative microbe.

6. The method of claim 4 wherein the microbe is a gram-positive microbe.

7. The method of claim 4 wherein the first plasmid comprises a conditional origin of replication, and wherein the conditional origin of replication is pir-dependent or TrfA-dependent.

8. The method of claim 4 wherein the first plasmid comprises a nucleotide sequence selected from the group consisting of nucleotides about 1-about 2,060 and about 4,155-about 6,664 of SEQ ID NO:1, nucleotides about 1-about 2,060 and about 4,158-about 6,668 of SEQ ID NO:2, nucleotides about 1-about 100 and about 1,135-about 3,663 of SEQ ID NO:3, SEQ ID NO:4, nucleotides about 1-about 100 and about 1,031-about 3,534 of SEQ ID NO:5, the nucleotide sequence SEQ ID NO:6, nucleotides about 1-about 100 and about 1,135-about 3,843 of SEQ ID NO:7, SEQ ID NO:8, nucleotides about 1-about 148 and about 3224-about 5739SEQ ID NO:9, SEQ ID NO:10, SEQ ID NO:11, SEQ ID NO:12, nucleotides about 1-about 547 and about 1,238-about 3,695 of SEQ ID NO:13, nucleotides about 1-about 255 and about 1,551-about 3,653 of SEQ ID NO:14, SEQ ID NO:15, SEQ ID NO:16, nucleotides about 1-about 129 and about 1,426-about 3,527 of SEQ ID NO:17, SEQ ID NO:18, nucleotides about 1-about 2,060 and about 4,157-about 6,742 of SEQ ID NO:19, nucleotides about 1-about 100 and about 2,195-about 4,782 of SEQ ID NO:20, nucleotides about 1-about 332 and about 3,408-about 5,948 of SEQ ID NO:21, nucleotides about 1-about 471 and about 3547-about 5750 of SEQ ID NO:32, SEQ ID NO:33, nucleotides about 1-about 445 and about 3521-about 5771 of SEQ ID NO:34, nucleotides about 1-about 445 and about 3521-about 5771 of SEQ ID NO:35, nucleotides about 1-about 615 and about 3691-about 5989 of SEQ ID NO:36, nucleotides about 1-about 443 and about 3519-about 5814 of SEQ ID NO:37, nucleotides about 1-about 829 and about 3905-about 6200 of SEQ ID NO:38, nucleotides about 1-about 397 and about 3473-about 5706 of SEQ ID NO:39, SEQ ID NO:40, SEQ ID NO:41, SEQ ID NO:42, SEQ ID NO:45, SEQ ID NO:46, and a combination thereof.

9. The method of claim 4 wherein the second plasmid comprises a nucleotide sequence encoding an integrase polypeptide, wherein the nucleotide sequence has at least about 80% identity to a nucleotide sequence selected from the group consisting of nucleotides 908-1981 of SEQ ID NO:23, nucleotides 937-2079 of SEQ ID NO:25, nucleotides 967-2217 of SEQ ID NO:27, nucleotides 974-2137 of SEQ ID NO:29, nucleotides 1034-2104 of SEQ ID NO:31, nucleotides 1542-2612 of SEQ ID NO:43, and nucleotides 1371-2441 of SEQ ID NO:44, and wherein the percent identity is determined by a BLAST2 search algorithm using default parameters.

10. The method of claim 4 wherein the polynucleotide is a first polynucleotide, further comprising repeating the introducing and detecting steps to integrate into the genomic DNA of the microbe a second polynucleotide.

11. The method of claim 4 further comprising excising the first plasmid from the genomic DNA of the microbe.

12. The method of claim 11 wherein the coding sequence encoding the integrase is a first coding sequence and the microbe is a first microbe, wherein excising comprises providing genomic DNA obtained from the first microbe comprising a single copy of the first plasmid integrated into the genomic DNA of a microbe, and introducing the genomic DNA to a second microbe, wherein the second microbe comprises a second coding sequence encoding an excisionase.

13. The method of claim 4 wherein the polynucleotide comprises a coding sequence.

14. The method of claim 13 wherein the coding sequence is obtained from a prokaryotic organism or a eukaryotic organism.

15. The method of claim 14 wherein the polynucleotide to be integrated comprises a transcriptional fusion or a translational fusion.

16. The method of claim 15 wherein the first plasmid comprises a nucleotide sequence selected from the group consisting of SEQ ID NO:9, SEQ ID NO:21, SEQ ID NO:32, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:45 and SEQ ID NO:46.

17. The method of claim 12 wherein the second plasmid comprises a nucleotide sequence encoding an excisionase polypeptide, wherein the nucleotide sequence has at least about 80% identity to a nucleotide sequence selected from the group consisting of nucleotides 910-1128 of SEQ ID NO:22, nucleotides 899-1117 of SEQ ID NO:24, nucleotides 903-1139 of SEQ ID NO:26, nucleotides 932-1093 of SEQ ID NO:28, nucleotides 935-1285 of SEQ ID NO:30, and a combination thereof, and wherein the percent identity is determined by a BLAST2 search algorithm using default parameters.

18. An isolated polynucleotide selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, and SEQ ID NO:48.
